(19) United States
(12) Patent Application Publication
(10) Pub. No.: US 2015/0299795 A1
(43) Pub. Date: Oct. 22, 2015

(54) CANCER-ASSOCIATED GERM-LINE AND SOMATIC MARKERS AND USES THEREOF

(71) Applicants: The Broad Institute, Inc., Cambridge, MA (US); Trustees of Tufts College, Boston, MA (US); North Carolina State University, Raleigh, NC (US); Regents of the University of Minnesota, Minneapolis, MN (US)

(72) Inventors: Kerstin Lindblad-Toh, Malden, MA (US); Noriko Tonomura, Belmont, MA (US); Evan Mauceli, Roslindale, MA (US); Jaime Modiano, Roseville, MN (US); Matthew Breen, Apex, NC (US)

(73) Assignees: The Broad Institute, Inc., Cambridge, MA (US); Trustees of Tufts College, Boston, MA (US); North Carolina State University, Raleigh, NC (US); Regents of the University of Minnesota, Minneapolis, MN (US)

(21) Appl. No.: 14/404,059

(22) PCT Filed: May 30, 2013
(86) PCT No.: PCT/US2013/043323

§ 371 Date: Nov. 26, 2014

Related U.S. Application Data
(60) Provisional application No. 61/654,067, filed on May 31, 2012.
(60) Provisional application No. 61/780,823, filed on Mar. 13, 2013.

(51) Int. Cl. C12Q 1/68

(52) U.S. Cl. CPC C12Q 1/6886; C12Q 2600/118; C12Q 2600/156; C12Q 2600/158; C12Q 2600/16

(57) ABSTRACT

The invention provides methods and compositions for identifying subjects, including canine subjects, having an elevated risk of developing cancer or having an undiagnosed cancer. These subjects are identified based on the presence of germ-line allele(s) and markers and various somatic mutations.

### CROSS-REFERENCE TO RELATED APPLICATIONS

This application claims the benefit of U.S. Provisional Application No. 61/654,067, filed May 31, 2012, and U.S. Provisional Application No. 61/780,823, filed Mar. 13, 2013. The entire contents of each of these referenced provisional applications are incorporated by reference herein.

### FEDERALLY SPONSORED RESEARCH OR DEVELOPMENT

This invention was made with government support under RO1CA112211 and U54HG003067 awarded by the National Institutes of Health. The Government has certain rights in the invention.

### BACKGROUND OF INVENTION

Several types of human malignancies arise from cells that belong to the hematologic system, including peripheral blood cells, bone marrow, lymph nodes and lymphatic and blood vasculature. Identification of mutations associated with these diseases is helpful for developing diagnostics and treatments. Several candidate gene and GWAS studies of human B cell Non-Hodgkin Lymphoma (NHL), a type of hematologic cancer, have identified germ-line predisposing risk factors in human NHL patients (Conde et al, 2010, Wang et al, 2010, Smedby et al, 2011, Conde et al, 2011). These studies reported conflicting findings of association of polymorphisms in the major histocompatibility complex (MHC) to some subtypes of NHL, highlighting the challenges due to the heterogenic nature of subtypes of NHL and of the human population. Human angiosarcoma, another type of hematologic cancer, is very rare and accounts for 1˜3% of adult sarcomas. Angiosarcoma is a very aggressive cancer due to its ability to facilitate excessive angiogenesis. The rarity of this disease in humans is a limiting factor when undertaking a feasible genetic study of angiosarcoma.

Dogs also suffer from several spontaneously occurring cancers of the hematologic system, including lymphoma (LSA) and hemangiosarcoma (HSA, a cancer of blood vessel endothelial cells). These canine cancers are clinically and histologically similar to human Non-Hodgkin Lymphoma (NHL) and angiosarcoma, respectively. The domesticated dog is an ideal model species to study genetics of human diseases and non-human animal diseases, as each breed has been created and maintained by strict selective breeding, thereby causing the alleles underlying desirable traits and alleles predisposing the dog to specific diseases to become common within certain breeds. Golden retrievers, one of the most popular family breeds in the U.S., have a high lifetime risk of cancer, with over 60% of golden retrievers dying from some type of cancer. Two of the most common cancers in golden retrievers are LSA and HSA, with a lifetime risk of 13% and 25%, respectively.

### SUMMARY OF INVENTION

The invention provides methods for identifying subjects that are at elevated risk of developing certain types of cancers. Subjects are identified based on the presence of one or more germ-line and/or somatic markers shown to be associated with the presence of cancer, in accordance with the invention.

In one aspect, the invention provides a method comprising analyzing genomic DNA from a canine subject for the presence of a risk allele identified by BICF2G63035726 or BICF2G630183630, and identifying a canine subject having a chromosome 5 risk allele identified by BICF2G63035726 or BICF2G630183630 as a subject (a) at elevated risk of developing a hematological cancer or (b) having a hematological cancer that is as yet undiagnosed (e.g., morphologically undetected).

In some embodiments, the genomic DNA is obtained from white blood cells of the subject. In some embodiments, the genomic DNA is analyzed using a single nucleotide polymorphism (SNP) array. In some embodiments, the genomic DNA is analyzed using a bead array.

In another aspect, the invention provides a method comprising analyzing genomic DNA from a canine subject for the presence of a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1, and identifying a canine subject having a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1 as a subject (a) at elevated risk of developing a hematological cancer or (b) having a hematological cancer that is as yet undiagnosed (e.g., morphologically undetected).

In some embodiments, the genomic DNA is obtained from white blood cells of the subject. In some embodiments, the mutation is in a regulatory region of the locus. In some embodiments, the mutation is in a regulatory region of a locus selected from the group consisting of ANGPTL5, BIRC3, CD68, MYBBP1A, CHD3, CHRNB1, RANGRF, ZBTB4, and a locus comprising SEQ ID NO:1. In some embodiments, the mutation is in a coding region of the locus. In some embodiments, the mutation is in a coding region of a locus selected from the group consisting of ANGPTL5, KIAA1377 and TRPC6. In some embodiments, the mutation is in a coding region of TRPC6.

In another aspect, the invention provides a method comprising analyzing, in a sample from a canine subject, an expression level of a locus selected from the group consisting of ANGPTL5, BIRC3, CD68, MYBBP1A, CHD3, CHRNB1, RANGRF, ZBTB4, and a locus comprising SEQ ID NO:1, and identifying a canine subject having an altered expression level of a locus selected from the group consisting of ANGPTL5, BIRC3, CD68, MYBBP1A, CHD3, CHRNB1, RANGRF, ZBTB4, and a locus comprising SEQ ID NO:1 as compared to a control, as a subject (a) at elevated risk of developing a hematological cancer or (b) having a hematological cancer that is as yet undiagnosed (e.g., morphologically undetected).

In yet another aspect, the invention provides a method comprising analyzing, in a sample from a canine subject, an expression level of a locus selected from the group consisting of TRPC6, KIAA1377, PIK3R6, ANGPTL5, HS3ST3B1, and BIRC3, and identifying a canine subject having an altered expression level of a locus selected from the group consisting of TRPC6, KIAA1377, PIK3R6, ANGPTL5, HS3ST3B1, and BIRC3 as compared to a control, as a subject (a) at elevated risk of developing a hematological cancer or (b) having an undiagnosed hematological cancer. In some embodiments, the locus is TRPC6. In some embodiments, the altered expression level is a decreased expression level of TRPC6, KIAA1377, PIK3R6, ANGPTL5 and/or BIRC3 compared to control, and/or an increased expression level of HS3ST3B1 compared to control. In some embodiments, the locus is TRPC6.

In some embodiments, the sample is a white blood cell sample from a canine subject. In some embodiments, the sample is a tumor sample from a canine subject. In some embodiments, the control is a level of expression in a sample from a canine subject having lymphoma and negative for risk allele identified by BICF2G63035726 and risk allele identified by BICF2G630183630.

In some embodiments, the altered expression level is (a) a decreased expression level of ZBTB4, BIRC3 and/or ANGPTL5 compared to control, and/or (b) an increased expression level of CD68, CHD3, CHRNB1, MYBBP1A and/or RANGRF compared to control.

In some embodiments, the altered expression level is analyzed using an oligonucleotide array or RNA sequencing.

In another aspect, the invention provides a method comprising analyzing genomic DNA in a sample from a canine subject for presence of a mutation in a locus selected from the group consisting of TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, and identifying a canine subject having a mutation in a locus selected from the group consisting of TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, as a subject (a) at elevated risk of developing a hematological cancer or (b) having a hematological cancer that is as yet undiagnosed (e.g., morphologically undetected).

In some embodiments, the genomic DNA comprises a risk allele identified by BICF2G63035726 or BICF2G630183630.

In some embodiments, the genomic DNA comprises a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1. In some embodiments, the sample comprises (a) a decreased expression level of ZBTB4, BIRC2 and/or ANGPTL5 compared to control, and/or (b) an increased expression level of CD68, CHD3, CHRNB1, MYBBP1A and/or RANGRF compared to control.

In some embodiments, the genomic DNA is obtained from white blood cells of the subject. In some embodiments, the mutation is in a coding region of the locus. In some embodiments, the mutation (a) is a frame shift mutation, (b) is a premature stop mutation, or (c) results an amino acid substitution. In some embodiments, the hematological cancer is a lymphoma or a hemangiosarcoma. In some embodiments, the lymphoma is a B cell lymphoma.

In another aspect, the invention provides a method comprising analyzing genomic DNA in a sample from a subject for presence of a mutation in a locus selected from the group consisting of ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, or an orthologue of such a locus, and identifying a subject having a mutation in a locus selected from the group consisting of ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, or an orthologue of such a locus, as a subject (a) at elevated risk of developing a cancer or (b) having a cancer that is as yet undiagnosed (e.g., morphologically undetected).

In some embodiments, the subject is a human subject. In some embodiments, the subject is a canine subject. In some embodiments, the cancer is a hematological cancer. In some embodiments, the cancer is a lymphoma or a hemangiosarcoma. In some embodiments, the cancer is a B cell lymphoma. In some embodiments, the cancer is a hemangiosarcoma. In some embodiments, the cancer is angiosarcoma.

In another aspect, the invention provides isolated nucleic acid molecules. In some embodiments, the isolated nucleic acid molecule comprises SEQ ID NO: 2.

### BRIEF DESCRIPTION OF DRAWINGS

FIG. 1 is a flowchart depicting the data analysis used to determine SNPs associated with LSA and HSA.

FIG. 2 depicts the loci associated with LSA, HSA, or both. FIG. 2A is a Manhattan plot depicting loci associated with LSA. P=p value. The data points located above the dotted line have a high log P value. FIG. 2B is a Manhattan plot depicting loci associated with HSA. P=p value. The data points located above the dotted line have a high log P value. FIG. 2C is a Manhattan plot depicting loci associated with LSA and HSA. P=p value. The data points located above the dotted line have a high log P value. The x-axis of the right-hand plot in each of FIGS. 2A-2C is the chromosome number, going consecutively from 1 to 38, followed by X, from left to right.

FIG. 3 depicts the loci associated with LSA and HSA located on chromosome 5. FIGS. 3A and 3B each show Manhattan plots depicting the two linkage disequilibrium regions where the top two SNPs were found. FIG. 3C shows the frequency of the risk and non-risk alleles for the 32 MB top SNP in the control dogs, dogs with HSA, dogs with LSA, and the combination of the HSA and LSA dog groups. FIG. 3D shows the frequency of the risk and non-risk alleles for the 36 MB top SNP in the control dogs, dogs with HSA, dogs with LSA, and the combination of the HSA and LSA dog groups. The left axis labels for both FIGS. 3C and 3D are, from top to bottom, 190 controls, B-cell_LSA_HSA, B-cell LSA, and HSA.

FIG. 4 depicts the LD regions on chromosome 5 associated with LSA and HSA. FIGS. 4A and 4B are two Manhattan plots depicting the two linkage disequilibrium regions where the top two SNPs were found. The X markers indicate an R-squared value of 0.8 to 1.0. The + markers indicate an R-squared value of 0.6 to 0.8. FIGS. 4C and 4D show the frequency of the haplotype blocks in the control dogs, dogs with HSA, dogs with LSA, and the combination of the HSA and LSA dog groups. The left axis labels for both FIGS. 4C and 4D are, from top to bottom, Control, B-LSA, HSA, and HSA or B-LSA. The figure legend for both FIGS. 4C and 4D is at the bottom of FIG. 4D.

FIG. 5 is a box plot depicting the expression levels (Y-axis) of genes in tumors from dogs having or lacking risk alleles for the chromosome 5 32.9 or 36.8 Mb regions. The left box plot for each gene is the expression from dogs with the non-risk allele. The right box plot for each gene is the expression from dogs with the risk allele. The circled dots indicate an FDR of <10⁻³.

FIG. 6 is a series of Manhattan plots showing the differentially expressed genes on chromosome 5. The X markers indicate an R-squared value of 0.8 to 1.0. The + markers indicate an R-squared value of 0.6 to 0.8.

FIG. 7 shows a diagram of a network of molecules involved in T-cell activation that are affected by the 36.8-Mb haplotypes. The molecules at the top from left to right are TNFRSF18, GZMK, and CD8B. The molecules in the next lowest row are GZMA, CD8, LAT CD8A, and CD151. The molecules in the next lowest row are Granzyme, Ige, TCR, CD3, ERK1/2, and CCL22. The molecules in the next lowest row are TNFRsF4, GZMB, TNFAP3, IL12 (complex), interferon alpha, FC gamma receptor, KLRC4-KLRK1/KLRK1, and CCL19. The molecules in the next lowest row are TLR10, Tnf (family), IL12 (family), IL1, CCL5, chemokine, CXCR3, and RGS10. The molecules in the next lowest row are Tlr, Ifn, EOMES, and Laminin 1. The molecule at the bottom is Igg3.

### DETAILED DESCRIPTION OF INVENTION

The invention is based in part on the discovery of germ-line and somatic markers associated with particular cancers in canine subjects. The two canine cancer types studied were B-cell lymphoma (referred to herein as LSA) and hemangiosarcoma (referred to herein as HSA). These cancers were chosen for analysis at least in part because they are clinically and histologically similar to human B cell NHL and angiosarcoma. These cancers are also relatively common in canine subjects. For example, golden retrievers in the U.S. have a lifetime risk for developing LSA or HSA of 13% and 25% respectively.

The discovery of germ-line markers was made by genotyping “normal” canine subjects and those having these types of cancers, and identifying markers (or alleles) that associated (or tracked) with either or both of the cancers. Surprisingly, this revealed a non-random association between certain alleles on chromosome 5 of canine genomic DNA and the presence of both of the cancers studied. Remarkably, two regions on chromosome 5 were found to contribute as much as 50% of the total risk associated with both cancers studied. Genes previously mapped to the regions of these alleles were sequenced and/or had their expression levels measured. A number of these genes were found to be differentially expressed in tumors from subjects carrying the risk alleles as compared to tumors from subjects that did not carry the risk alleles. Risk alleles are also referred to herein as risk-associated alleles. The differential expression pattern may be indicative of the downstream mediators of the increased cancer risk associated with the alleles on chromosome 5. In addition, a number of these genes were found to be mutated in their coding regions in tumors from subjects carrying the risk alleles.

Similarly, the discovery of somatic markers associated with particular cancers was made by genomic sequencing of tumor cells and matched normal cells from canine subjects, and then identifying differences between the genomic sequences. A variety of somatic mutations were discovered in tumor cells that were not present in normal cells. In some instances, the observed somatic mutations affected gene products (e.g. frameshift mutations).

The invention therefore provides diagnostic and prognostic methods that involve detecting one or more of the germ-line and somatic markers in canine subjects in order to (a) identify subjects at elevated risk of developing a hematological cancer such as LSA or HAS or (b) identify subjects having a hematological cancer that is as yet undiagnosed (e.g., because it is morphologically undetectable at that time). Accordingly, the methods can be used for prognostic purposes and for early detection. Identifying canine subjects at an elevated risk of developing such cancers is useful in a number of applications. For example, canine subjects identified as at elevated risk may be excluded from a breeding program and/or conversely canine subjects that do not carry the risk alleles may be included in breeding program. As another example, canine subjects identified as at elevated risk may be monitored for the appearance of certain cancers and/or may be treated prophylactically (i.e., prior to the development of the tumor) or therapeutically (including prior to a detectable tumor). Canine subjects carrying one or more of the risk markers may also be used to further study the progression of these cancer types and optionally the efficacy of various treatments.

In addition, in view of the clinical and histological similarity between canine LSA and HSA with human NHL and angiosarcoma, the markers identified by the invention may also be markers and/or mediators of disease progression in these human cancers as well. Accordingly, the invention provides diagnostic and prognostic methods for use in canines, human subjects, as well as others.

The invention refers to the germ-line and somatic markers described herein as risk-associated markers to convey that the presence of these various markers has been shown to be associated with the occurrence of certain cancer types in accordance with the invention. The germ-line markers may also be referred to herein as risk-associated alleles. The somatic markers may also be referred to herein as risk-associated mutations. These various marker types will be discussed in greater detail herein.

### Elevated Risk of Developing Cancer

The germ-line and somatic markers of the invention can be used to identify subjects at elevated risk of developing a cancer such as a hematological cancer. An elevated risk means a lifetime risk of developing such a cancer that is higher than the risk of developing the same cancer in a population that is unselected for the presence or absence of the marker (i.e., the general population) or a population that does not carry the risk-associated marker.

### Germ-Line Markers

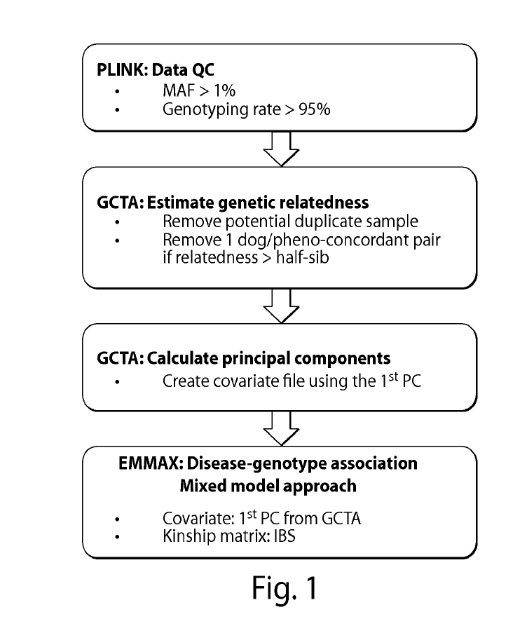

The germ-line markers associated with HSA and LSA in canine subjects were identified through genome-wide association studies (GWAS) of 148 HSA cases, 43 B cell LSA cases, and 190 healthy older control golden retrievers. The analysis was performed using single nucleotide polymorphism (SNP) arrays customized for canine genomic DNA analysis. Such arrays are commercially available from suppliers such as Affymetrix and Illumina (Illumina 170K canine HD array). Such arrays can be used to analyze genomes for polymorphisms (or alleles) in a population. Each polymorphism will have an expected frequency based on the general population. These GWAS studies identify polymorphisms in a particular subject that are present at a disproportionate frequency (otherwise represented by a “P value” that differs from the expected P value for the polymorphism in the general population). The data set so obtained was also controlled for population stratification given the known high levels of encrypted relatedness and complex family structures in canine populations such as golden retrievers. The data analysis algorithm is shown in FIG. 1.

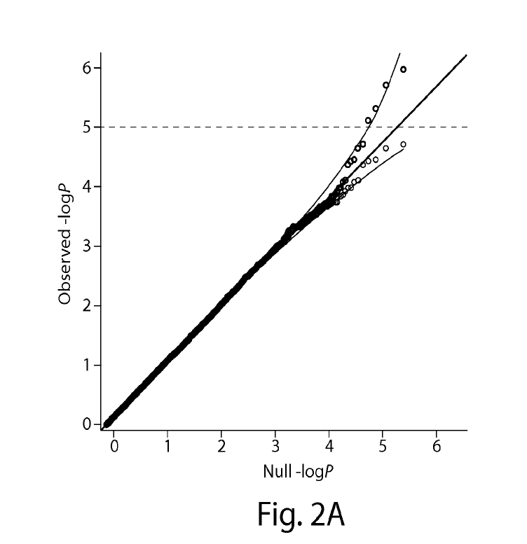

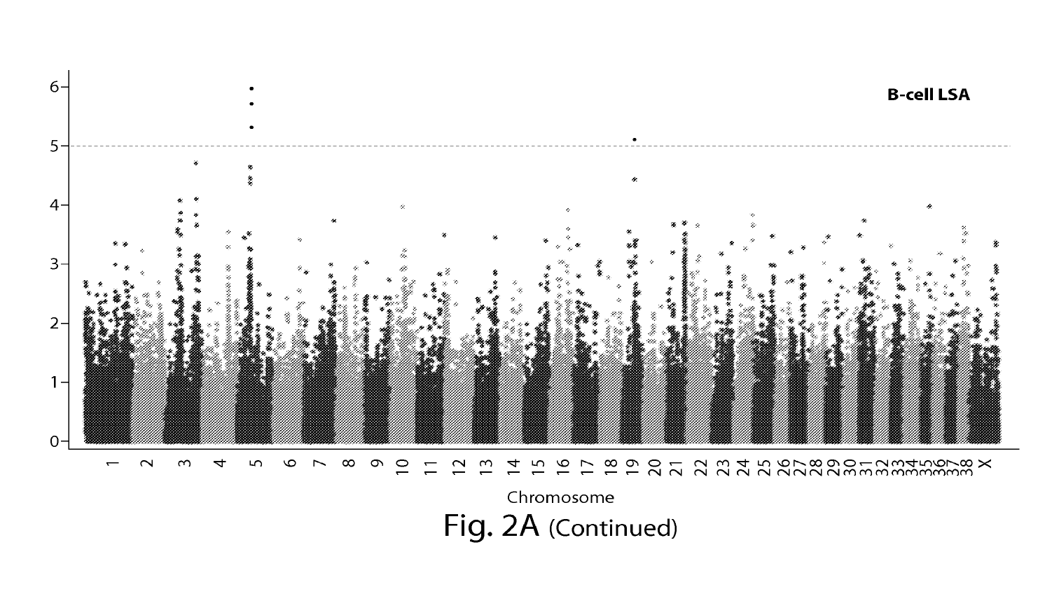

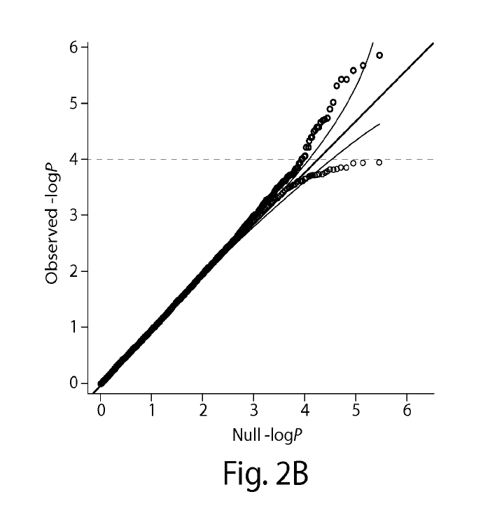

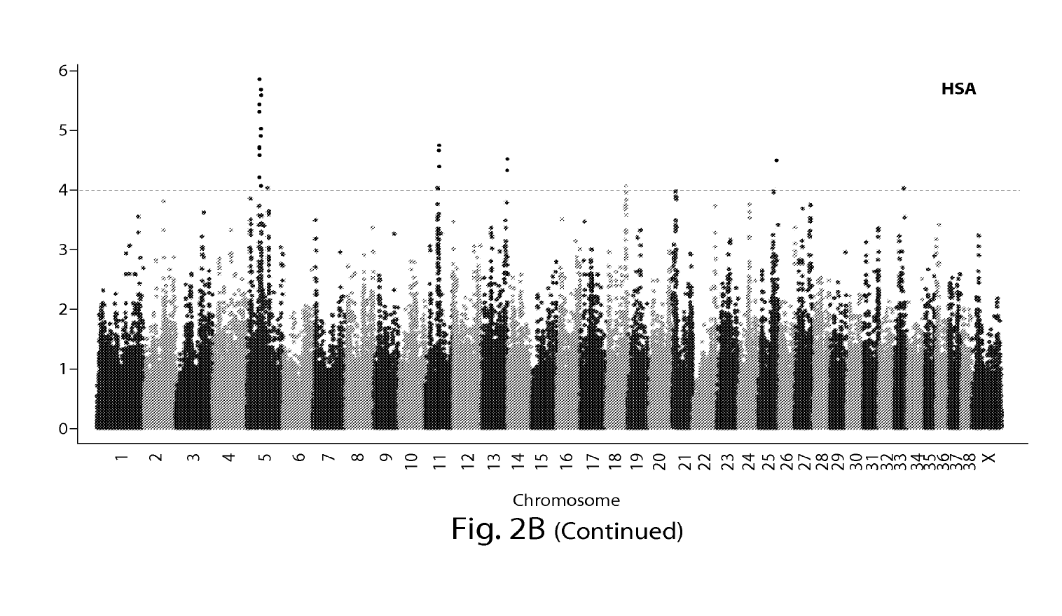

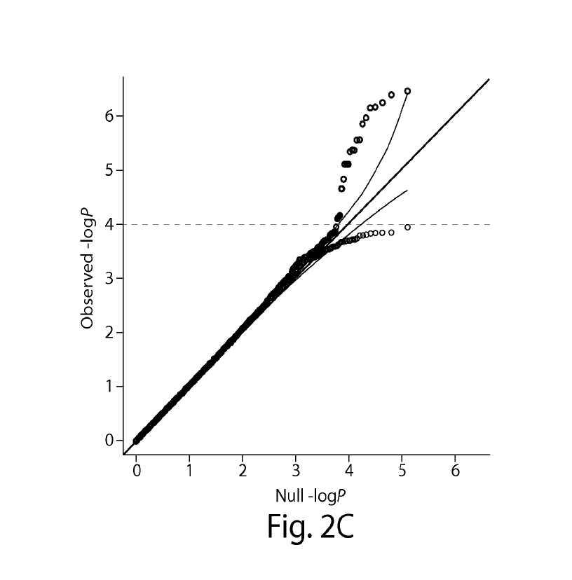

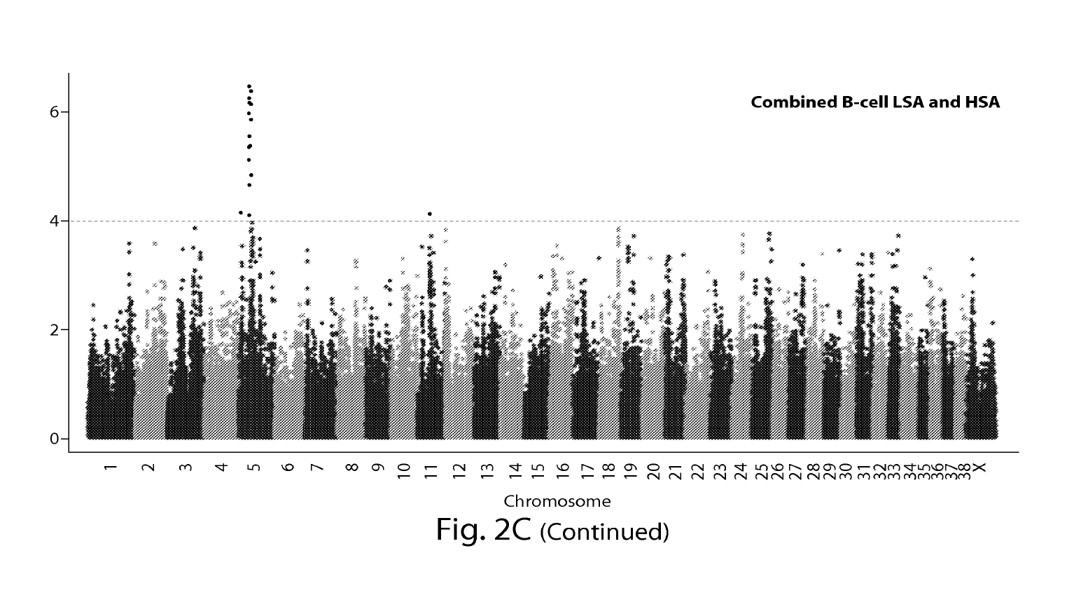

This analysis revealed the presence of one or more regions within chromosome 5 that were disproportionately represented in the subjects having LSA and HSA compared to the control “healthy” subjects, as shown in FIG. 2. Each dot in the Figure represents a different SNP in the SNP array used in the analysis. The nucleotide sequences of the top SNP are provided herein as Table 1. The top SNPs are BICF2S23035109, BICF2G63035383, BICF2G63035403, BICF2G63035476, BICF2S23317145, BICF2P1405079, BICF2G63035510, BICF2G63035542, BICF2G63035564, BICF2G63035577, BICF2G63035700, BICF2G63035705, BICF2G63035726, BICF2G63035729, BICF2P93507, BICF2G630183626, BICF2G630183630, BICF2G630183805, BICF2P267306, BICF2P1337948, and BICF2P858820.

TABLE 1
Nucleotide sequences of SNPs

| SNP ID | Chr | position | P value | Sequence |
|---|---|---|---|---|
| BICF2S2 | 5 | 11757453 | 7.07E−05 | TGGCCAGCTCTCCACCAGAGCGTCATCCTTGGAGATCCAGCCAAG |
| 3035109 | | | | GGAAGGAGAGAGACCAAGAAGCAAGATCCCTAAGTGAAGGTTG |
| | | | | GGCGCTAGAAAAAAATGTCCCAGGTAGCAGTAGCACCTTCTCTTT |
| | | | | GCCCTCTGTCCTTTTCCATCTAGACAACCCATTCCGAGCAGAGACC |
| | | | | TTGACAGTCGTGTTCCCCAGC[G/A]AACCAAATACAGGGGACATC |
| | | | | CTTTATCCCTGAAAGTGTCCCCTTGAACAATGTAGGGGACAGAGC |
| | | | | AGACACTAGGAAATATTTCTGAGCAAACAAGAATGATAGAGCAA |
| | | | | ATAGATGAATGCCTGAGTATCTGCCTGGCCCAGAAAGCAGCTGT |
| | | | | AGGAGAGAATGGCCCGAGAACCCCCCATCCCCACCCTCCACAGA |
| | | | | CTG (SEQ ID NO: 3) |
| BICF2G6 | 5 | 32622509 | 2.23E−05 | CCCTATTTTTCTTAACTTTCCTATTTCATTTATTTCTGATTACCTCGG |
| 3035383 | | | | GCGCTTGAGTTCATGTTAACTAGTTTCAACTGTTTAAAAAATTAAT |
| | | | | TTTGTGGAAAATATGTGTCTGTGTGTGTTTCAATAATTAGTTGCTG |
| | | | | GACAATGAGAAGAACGAAGATTGAAGCTGGCAATGACTGGTTAA |
| | | | | GAAACTGCTAAGTGGT[T/A]CTGCTCTTTGGAATGTATGGGGTTG |
| | | | | AGAAGCAGTAAATAAGAACTCTTCATGATTTTCAGGGTCATGGGC |
| | | | | TACTATTAACCATCATTCCAAAGACCTTCAGTGGCTGGAGCTTTAT |
| | | | | TCTTTTTTTTCTCTCTTTTTTAAATTTATTTTTTATTGGATTTCAATTT |
| | | | | GCCAACATATAGCATAACACCCAGTGCTCATCCCG (SEQ ID NO: |
| | | | | 4) |
| BICF2G6 | 5 | 32632285 | 2.23E−05 | AATAAAATATATGGCAGACCATTCATTTAATGTAGCCTTTGAAAA |
| 3035403 | | | | GAGAAAACACAGAGGCAACTAAACGGAAGCATGAACTGAACATT |
| | | | | CCACTTTCCGTGAGGTGGAGGGGGCTGAGGTGCACTCGGCAAAA |
| | | | | GCTGATAGAGTCAATGGAACAAGCTCTGAATTCAGATGCTCTTAC |
| | | | | TTGACATTAGGACAGCAGTGTC[T/C]AGGACACCAATACATGATT |
| | | | | CCCTCCTATGGTAGCTCATGTCCAAAGACAGTCGCAATGAACCAT |
| | | | | GATTATACAGTCCTCTCTGCTTTAATCAGCCTGGTCCTGTGACTCT |
| | | | | CTTCTAATCAATAGAATTTTATAGAAGATGCTAGATTCAAAACTCT |
| | | | | GCAGCTTCCAACTTGATCTTTTAGCATGATTGCACTAGGAAAAG |
| | | | | (SEQ ID NO: 5) |
| BICF2G6 | 5 | 32708612 | 4.61E−06 | ACCCTCCCCAAATCGAACTCAAAACAAAACAAAACAAAACAAAAC |
| 3035476 | | | | AAAAAACCCAGAAAGGACTGCCCTGGAAGTCCTTGTCTGATTTTC |
| | | | | CCAACCTTTTCCTTCCAGGTGTATACCAAATGGACCAGGCTTCCGT |
| | | | | GACTTGCCCACAAGTGCCCCCAAACATGGTCATCAGGGAGTATCC |
| | | | | CAGCTATAAAGAGAATATC[G/A]TTTATGCTGGGCCTACTTCCCG |
| | | | | AGCCTCTCAATCCACTGCTAATGTCCAGCGTAAGAATTATTCAACC |
| | | | | TCACTCAGTTTGAGCCATGGTGTTTTTTATGGTTCTACACCTATCC |
| | | | | TAAAAGTAAATTTGCCTCATTTCTTGCTATCCTGGCTAGTCTTCAA |
| | | | | TCCTTCCTCTCAGAAGACTACTGAGTAAGAAACTCTTTCA (SEQ ID |
| | | | | NO: 6) |
| BICF2S2 | 5 | 32725862 | 4.33E−06 | GAAATTTTTCATGTTGACTATTTCAATAAATGATTTAGGGCTCCTT |
| 3317145 | | | | AATTTGTAAGGTACAAAGATACGCTTGAATTACTGGGAGGATAA |
| | | | | GGATGTTTATTTTCAGAATATTAAGCGGAAAACTTCCAAATATCA |
| | | | | AAAAGGAGTTATAATAGCTTTTAAACTGAAATTTAGAAATACCCA |
| | | | | GATAAATGAAATTATAAGAT[G/A]TAGATGTATTTATGTATTTAAT |
| | | | | CAATTGTTTCCCAGACCCATATTATTGATTCTTCAGTTTTGAACAA |
| | | | | CTTCTAAAGATTCTTGAGTTTCTGTTTCCTCATCTAGAGAATGTTT |
| | | | | ATATACTCCACTCACTGTGAGGTTTACATGATAACTTTAATTTAAA |
| | | | | AACAAACACACAAACAGCTCATTGTCTATTATAAATGCTA (SEQ |
| | | | | ID NO: 7) |
| BICF2P1 | 5 | 32757545 | 7.91E−06 | ATGGAAAGCTATGTCTATTTTTGAACATTAGCTTTTTTTCACTAAT |
| 405079 | | | | TTTTAATCGCAGACTACTTTTCCCTGAATATAACAAGTAGAAAGTA |
| | | | | GTTAATTTTCCTGTAGAAAGGTCTCTGAAGATTTCACTCATCCTTT |
| | | | | CTTGTCTCTCTAATCTCCCAAATGACTGGAAATAATGAAGGAAGA |
| | | | | GATGCTGGTACTCCTCT[T/G]GGGTAATGATCTCATGACTTCCAA |
| | | | | GGGTGGGTTTGCCGTCATAATAGTTGGGGACAAAAGTCAGGGTG |
| | | | | GTAACTGGGGGTCGAATAAGATCTGGCAATCTTGAGAATGTGAA |
| | | | | GTAATTTGGAATATAGCATGCTCACAGAGTGGGTATTTTATCCTG |
| | | | | TTGGGTAGAAAAGACCATCAGGTGTGCTGGTGTCTGTTACTCT |
| | | | | (SEQ ID NO: 8) |
| BICF2G6 | 5 | 32757807 | 7.91E−06 | CAGGGTGGTAACTGGGGGTCGAATAAGATCTGGCAATCTTGAGA |
| 3035510 | | | | ATGTGAAGTAATTTGGAATATAGCATGCTCACAGAGTGGGTATTT |
| | | | | TATCCTGTTGGGTAGAAAAGACCATCAGGTGTGCTGGTGTCTGTT |
| | | | | ACTCTTGGGAGCCCCTAGTTTAAAGACTAGGAGGCTAGCTCAGA |
| | | | | GTTGAGGAGATGCCTTTTTTTC[C/A]AGAGTAAGAATAATTTTGA |
| | | | | TTTTCAGTATTTCCAGTGGAGCAGATCCCTCCAGAAAAGTCGTGA |
| | | | | GAGCTGCATGTCAATTGCTGTTATCCCAATGGTATTTAGAACATTT |
| | | | | TCTTGATTAATTACAAGAAAATTTGGCCTGGGAGGGGGCTGCAT |
| | | | | CCTGCTGCTTTCAGGAGCCATGGCCTGGGAGCACAATTCCAGCA |
| | | | | GT (SEQ ID NO: 9) |
| BICF2G6 | 5 | 32771537 | 7.91E−06 | CGTGACCTGAGCTGAAATCAAGATTTGGTGGCTTAACCAACTGA |
| 3035542 | | | | GCAGCCCAGGTGCCCTGACAGTGAGTTGTAAAGCAAGAAAGCAA |
| | | | | GCTTGTGATTAGTCCAGTGAGCCTGTTAAGTAGAGGTTCATTAAA |
| | | | | AGAGCTTGTATGAACTGAATTATAGAGTATGCCATGGCTTTCTCT |
| | | | | TGCAGAAAATATGAAGTGCATA[C/T]AACTATGAAATGTGGCTAC |
| | | | | TTCTATGCAAGAATGACATTCAGAATAAAATTATTCATCAACAAC |
| | | | | AGCTATCCAGATGTTTTAGTTTGTAAAGACATAATTTTAATGACC |
| | | | | GAATATTTCAGTTTTCATTTGAATGTTCCAATTTTTTTGAATACGCC |
| | | | | ATTTCATTTTCAGTGCTAAATAAATACTTGTTCAGTTTTTAAAA |
| | | | | (SEQ ID NO: 10) |
| BICF2G6 | 5 | 32787898 | 2.83E−06 | CTCAGCAGATGCATCACATTTGGCGCACAAGTCCCAAGTCAGGTC |
| 3035564 | | | | CAGTGACCAACGGAGGAAGACCCTTGAGGGCTAGGATTGTGCCT |
| | | | | ACTGTTCTCCTACCTGTTGGCTCCACCTGCACTCATACCAAGCACT |
| | | | | TCTCTGACCCTCGCTGCCACCAGCTCTGCCCCATCCTGGTTGCCGT |
| | | | | CATCTCTGAAGACTGGCAG[A/G]ACCCATGAGACTTAAGAACTTT |
| | | | | CCCAAGCTTCTGCTTTGCATTGGCCTAAATCTCTGTAATTTAAAGG |
| | | | | ACTTCTTCCAACTCCCATTCCCTGACCTCATCTGGTGTCACTTTATC |
| | | | | ATCCAACATAAACACTATTTCTCACCTCAGCTCCCCACACACTGTT |
| | | | | TCTCTCTTGTGTGGCTTTATTTCTGTTTCACTTATTCTC (SEQ ID |
| | | | | NO: 11) |
| BICF2G6 | 5 | 32804686 | 2.83E−06 | ATCAACAAGGAGTGTAGAAGGAAAAAATTCAGGTGAGGGGACA |
| 3035577 | | | | GCCAGTGAACTGCGCTGACATTCTCTCTGGAGATGTGTGATCCTC |
| | | | | AGGGTTTGTGCTGAGCCTGGCCTCCCCAGGAAGAGGACTGATGG |
| | | | | ATGTGCAGAGAGAAATCAACAAATACTCCAGTTTGTACAAAGTTG |
| | | | | AAGACTGAGGGGCCATTAAGACA[C/T]AGCTGCTTTTTGCCTAAA |
| | | | | ACCTTTTGCTAAATTTTGAAATTGTCTGTGTCAAGAATAGGATATA |
| | | | | TTTTCCCCTTGAGAGTCGTGGTGTATACTAACAAGTGCTTTGAGA |
| | | | | AATCTTTCTGAGACAAAAGCTGTTTAACTTCTGTATCCTGTATTTT |
| | | | | CCCAAACTTACTTGCTTAAGGTGTTCTTAATTCATAACAAAAGAC |
| | | | | (SEQ ID NO: 12) |
| BICF2G6 | 5 | 32876294 | 6.89E−07 | TATGTGGCTGTCACTACAGCAATGACTTGTTTTTGGCATATGTCAC |
| 3035700 | | | | ATCATTTCAGAGAAACAAAAATTAATCTGAAATCTTTCCTTAAGG |
| | | | | ATTAATTCTATGTATATAAGAAGGAAAGTCAAGGCAATGGAAGC |
| | | | | AGGCAAATTTAGGCAATAAGGATCCTGGGATTAGTGAGAGAATG |
| | | | | TCCAACAAATCCTCCAAGGGA[G/T]GCTCAAAGCAATAGGCTCTG |
| | | | | CTTCAGCAGGATGGTAAAGGTCACCCTTGCTTATTTTTGCTGCTTC |
| | | | | ATGGAGAGAAGCAGTAGACTTAAATATGTTTAAGGGCTTAAAAC |
| | | | | AGAATGAAGTTGAGACTGTCTGGGTTATCTGACTGCTGGACTTCT |
| | | | | TCAGTGCTGCTGGATTCTAAACAGAGTCCATCTGTGTCAAGTGTT |
| | | | | (SEQ ID NO: 13) |
| BICF2G6 | 5 | 32879166 | 5.77E−07 | TTTAACTTCTATGCTTCAAAATCTTTACAGTCCATGAGAAAAGCAC |
| 3035705 | | | | AGCAGAAGTTAAAGCTACCCAGGGATTCCCAGATGAGGACCTAT |
| | | | | TAACTGTGAGAATGTGCTCCTTTGTTATGTTTCTCTCAGAGAGTG |
| | | | | AGTCCACCTCAGGTTTCCAGAGTGTGGATCCCCTCCCCCGAATCA |
| | | | | CAGCGGCTGCTTGGGGTCTG[G/T]AATCCCCCATCCACTCTGTAG |
| | | | | GCAAAAACCCTGTTAAGCAATGTGGGGGACACAGCAGCCAGTGG |
| | | | | GGGGTTTGTCTTAGGTTAGGGCCCCAGATCTGATCATCTTACAAG |
| | | | | TCTTCTTGACATATACAGTAAATACATGGCTTTGCTTTCAGGCCAG |
| | | | | GAAAATCTTGAGAACACATGTCAATATTTTGTAGAAAATTATTT |
| | | | | (SEQ ID NO: 14) |
| BICF2G6 | 5 | 32901346 | 3.52E−07 | AAATATTTTTCTTTATTATTTCAGCTTTTAGGGGAATACTTAGAAT |
| 3035726 | | | | GGCATTATACACCTGAAGATTACATATTAAAAAATAAAAGTTCAC |
| | | | | CTGACTCTTTCTCTAGAGGTTTTATGGTTTTTAAAATGACATTCAA |
| | | | | TTTCTTAATGCATCTCATTTACTTTATGGCAAAGGTAGAGAAAGA |
| | | | | AATCTCTTACCCACTTCC[C/T]TTTATTCAGTATGGCTTCATTTTCC |
| | | | | CCAATGAGTTATGTCCTTTATCATACCATAAAATCTTATATCCTTA |
| | | | | AGTCTTTGACTGAACTTTCTATTATATTCTTTTAATGTTTCCATTTT |
| | | | | GTGAATACCCAACTATTTATTGTGGTTTTACTAAATCATTTAATGT |
| | | | | CTTATAGAGCAAGTGCCTTTCACTGCTTTTTAAAAA (SEQ ID NO: |
| | | | | 15) |
| BICF2G6 | 5 | 32902463 | 1.10E−06 | GTTCATTTAATTTACCAAAGTTAACATTATTCACTTTACAGCATAT |
| 3035729 | | | | GTAGAAAATTGAGGTCCAGGCTGTATTTGACTACTTCCAGTGATA |
| | | | | AGAAAATAACTACATATAAGGCAGTTCCATCCATTCTTGATATGT |
| | | | | CTGCATTTCCTGAGCCAGGGTGACTCCACTCCATAACTCAGCAGT |
| | | | | GCTCTCAACTGTCCTCTGA[C/T]TGTTTATGAACCATTCCTCTGTTA |
| | | | | CCCAGTCTGTATTTGTTAATCTTGCTGCCAAATATATTACATGATG |
| | | | | CCCTTGGTCAGTAGTTTACTGAAAGGATTCTAATCATTCTACCAAA |
| | | | | AGACAAGTTAATTTAGTCCAGCATGACTTATCCTAAGCAAAGCCG |
| | | | | CGGTGATCACAGTGCTTACACAATATCCGTATAATAATA (SEQ ID |
| | | | | NO: 16) |
| BICF2P9 | 5 | 33009401 | 7.98E−05 | GGTTCTCAGTCTTGCTCTCCCCTCTGTTGCAGGTGAGCTGAGCCTC |
| 3507 | | | | AGGGTCTGGAAGCCTCTTCTGCCTCCCCTGCTCCTATTCCCCATTA |
| | | | | TCTTCCAGAGGCATGATTCTGGATACATCTCATACTTCTAACTGTC |
| | | | | TTGGCGTTTGGTTCCTAGAAGACCAAAGTAACACAACTATCCTCT |
| | | | | TTACTTTGCTTGGCCCC[A/G]TAAGCCTGTGATGAACAAAACTTG |
| | | | | GGGCACGTGCCAGACACGTATTCCTGGGAGAATGTTTTTTAAAG |
| | | | | CAACTGTTTATATTTCAAATCATTTTTGCCTATTGTGGACTCCCACT |
| | | | | GGAAATGTGTGGCTCTGACAGGTACCAAGGAAAGTTATGCGCCC |
| | | | | TTACCCAACGTGGGTAGCTTTTGCTTTCTTTTTCATAAAGT (SEQ |
| | | | | ID NO: 17) |
| BICF2G6 | 5 | 36845402 | 1.43E−06 | GACCCTGTGACTCTCCTTCCTGACAGAAGCCTTGGGGAAGGCCCA |
| 30183626 | | | | AGACGGGAAGGAAGAAGCACCAGCGAGGCCAAGGACAGCAGG |
| | | | | AAGGAGCTAGAATGATTGCAGCTTCGGCCGCGATCCGCTGGATG |
| | | | | CAGACGGGCCGGCTGTGACACTCCCTTCCCCGCATCACAGGTCCT |
| | | | | GATCTTGGACCACAGCCGCATCTC[C/T]AATGCATGGTGCATCCA |
| | | | | AAGGAGGTGCTCGGTCATCACATGTGGCTCACCACGGCAGCCTG |
| | | | | CCCTCCCAGAGGGTGTCCTGGAACCGGCCCTCTGCAGAGCTGGT |
| | | | | TTCAAAGCCCGGGCAGCCCCTCTGCGAGCCGCCTTCCTCCGGCAC |
| | | | | GGTGGGTGAGGAAATGAGAGAGGGAATGTCTAATGATTGGTTC |
| | | | | CTTATGG (SEQ ID NO: 18) |
| BICF2G6 | 5 | 36848237 | 4.20E−07 | TGGGCCTCACCCATAGAATGGGGATCATAGTAGTATGTATATTTG |
| 30183630 | | | | TTGGGTATGGTACCTATAACCCAGTCATGCTCAGTAAACATCTGC |
| | | | | TTTTCCCATTACTAGGGCTTCACCAGGCATGTTTCATGGTGTGCCT |
| | | | | ATAGTCCCTTGAAATGGGCTCTTTGTTGACCTAGACTCTGGTTGA |
| | | | | GGGCAAGCCCTGGCAGCTG[C/T]GGTTTTTACCTCATGATCCTAC |
| | | | | CCATTGAGCCATGGTGACTTGGGCACATAGAGGTGACCCAACCC |
| | | | | ATGGGCTGGCCAGCAGTTTCTGACTCAGCCCATGAAGCTGAGTT |
| | | | | GAGTAGAAAGATTCTTTTCTCTTTTTGAACTAGGAAATAAGGAGC |
| | | | | CATGCAGACTTGCTAGATGTCCTTAGGATAAATTCACCTTTTTTG |
| | | | | (SEQ ID NO: 19) |
| BICF2G6 | 5 | 37081986 | 1.49E−05 | GGCTCTGTGCTCCTAGACCATACTTGTGGAAATCACTAATGATGT |
| 30183805 | | | | ATGCTATAGCTCCTACCAACTGTGGAACATAACTGGTAAGTCCTT |
| | | | | CTGGAGTGTGGAAGTGAGAGAAATCACTGGCGGCCGAGGCACT |
| | | | | CAGATTTGACAGGACTAGGCCAAGAGATTATATTCTGGGCTGAA |
| | | | | CTGCAAGATTGAGAGGCAGGAGG[A/G]AGAGGCACATTCTGGA |
| | | | | CTGGGCCAAAGACACAAGAAACAAGCAAAGGTGAAAGGAAGGA |
| | | | | AACGGGCCTGGCACATTGGGCTGAGTGGCCCTAGGTAGGAGGA |
| | | | | TACAGTATCACAGGGTCACAGATGCGGGGAAGCTAGTTTCTGCA |
| | | | | GAGACTCGAACACCAGGATCATCGGGTGAGACTTGACAAGAGG |
| | | | | GCTTTAGAGAT (SEQ ID NO: 20) |
| BICF2P2 | 5 | 37099612 | 4.33E−06 | GCCCAAACTTTTTTTTAATTTTATTTTATTTTTTTTTAAGGACAGTC |
| 67306 | | | | TGTTATTCTAGATCTGCTTTAATTTCATGCAACAGTGATAACTAAG |
| | | | | AGTAAGTAAGTACTCGTAAGTAAGATTTCTGGTATGGCACCCACA |
| | | | | GTACCCTCCATGTTGGCCCCAGTTTCAGATATTCCATTATATTGTC |
| | | | | CCTCATGAGAGATCCT[A/G]CAAATTCCAATTTGACATCCAAGTG |
| | | | | ACATCTACCAGGAGGGCCTTGGAGAAGCTGATTTTTCTTTTTAAT |
| | | | | TTTGAAAGTACTCAAAATAAGAATTCAAATGAGAAGTATATTTTT |
| | | | | ATTCAAATGAGAATGTGTACAAAATAGTTATCGAGCACTTATTAT |
| | | | | GTGCATAACACTGGAGGCCAAAAAAAAAAAAAAAGAGGAA |
| | | | | (SEQ ID NO: 21) |
| BICF2P1 | 5 | 37111219 | 7.29E−07 | CAGGAGCCCAATGCAGGACTCAATCCCAGGACCCCAGGATCATG |
| 337948 | | | | ACCTGAGCCCAAAGCAGACGTTCAACCATTGAGCCACCCTAGAGT |
| | | | | CCCTGTGTCTCCTTTTCTTGTCTTGTGTTGTGTCGTGATCATGTTTT |
| | | | | GTGGTTGTACCTTCCCCTCCCTGACTTCACATGACTTGGAAACTAT |
| | | | | TCATGGTATTGTTTGTTA[G/T]TTATCAATCTTTAAGTCATAAGTA |
| | | | | TGTATATTTGATATAATAATTTATGATTATGATATTGTTTCTAGTTC |
| | | | | TTTCTAGATATTGCCTGTCTGTTAATTCATTGGTATCATAGTTTCCT |
| | | | | TTACATTTTTAAATATTTTATTTGTTTATTTGAGAGAGAGTGAGAG |
| | | | | ACAGAGATAGTGAGAGCATGAACAAGGAGGAAAGGG (SEQ ID |
| | | | | NO: 22) |
| BICF2P8 | 11 | 40794422 | 7.57E−05 | TGATACATTTCTTACAGGATGGTTTTGTCATGTAGAAGCTCTTTTA |
| 58820 | | | | AAGCACTCCATCCTTATTTTCCCATTGATCATTTCTTTGCCTCCTTT |
| | | | | TCCCCCTTCTCTCCTCTAGAAATGTCCCTTTTTCTCTCACCATTATC |
| | | | | AGCACCCATTAACCTTCTAAGTAACACAATTGATTTTGACCTCTCT |
| | | | | TTGTGGTTTTAATT[T/C]ACCATAGGTGTCAGGGGTGTCATCTTTC |
| | | | | TTTTCTGTTTCCCCTGTGTTCCAGCCTGCTTGAGGGTGAATGCCCT |
| | | | | GGCAGGGTGTGCCCACATGCACTCACATATAACCCATAGACAGC |
| | | | | AACTCCAGGAACATATCAAACTGGATTTCTTAAGTCACTGGAGCC |
| | | | | TATGGGTGACTGTACGTATAGACAATATATTTTGAAT (SEQ ID |
| | | | | NO: 23) |
| BICF2S2 | 5 | 11757453 | 7.07E−05 | TGGCCAGCTCTCCACCAGAGCGTCATCCTTGGAGATCCAGCCAAG |
| 3035109 | | | | GGAAGGAGAGAGACCAAGAAGCAAGATCCCTAAGTGAAGGTTG |
| | | | | GGCGCTAGAAAAAAATGTCCCAGGTAGCAGTAGCACCTTCTCTTT |
| | | | | GCCCTCTGTCCTTTTCCATCTAGACAACCCATTCCGAGCAGAGACC |
| | | | | TTGACAGTCGTGTTCCCCAGC[G/A]AACCAAATACAGGGGACATC |
| | | | | CTTTATCCCTGAAAGTGTCCCCTTGAACAATGTAGGGGACAGAGC |
| | | | | AGACACTAGGAAATATTTCTGAGCAAACAAGAATGATAGAGCAA |
| | | | | ATAGATGAATGCCTGAGTATCTGCCTGGCCCAGAAAGCAGCTGT |
| | | | | AGGAGAGAATGGCCCGAGAACCCCCCATCCCCACCCTCCACAGA |
| | | | | CTG (SEQ ID NO: 24) |
| BICF2G6 | 5 | 32622509 | 2.23E−05 | CCCTATTTTTCTTAACTTTCCTATTTCATTTATTTCTGATTACCTCGG |
| 3035383 | | | | GCGCTTGAGTTCATGTTAACTAGTTTCAACTGTTTAAAAAATTAAT |
| | | | | TTTGTGGAAAATATGTGTCTGTGTGTGTTTCAATAATTAGTTGCTG |
| | | | | GACAATGAGAAGAACGAAGATTGAAGCTGGCAATGACTGGTTAA |
| | | | | GAAACTGCTAAGTGGT[T/A]CTGCTCTTTGGAATGTATGGGGTTG |
| | | | | AGAAGCAGTAAATAAGAACTCTTCATGATTTTCAGGGTCATGGGC |
| | | | | TACTATTAACCATCATTCCAAAGACCTTCAGTGGCTGGAGCTTTAT |
| | | | | TCTTTTTTTTCTCTCTTTTTTAAATTTATTTTTTATTGGATTTCAATTT |
| | | | | GCCAACATATAGCATAACACCCAGTGCTCATCCCG (SEQ ID NO: |
| | | | | 25) |

Further significant SNPs are listed in Table 2. The use or detection of any SNPS listed herein is contemplated.

The position (i.e., the chromosome coordinates) and SNP ID for each SNP in Table 2 are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The first base pair in each chromosome is labeled 0 and the position of the SNP is then the number of base pairs from the first base pair (for example, the SNP on chromosome 5 at position 36,417,176 is located 36,417,176 base pairs from the first base pair of chromosome 5).

TABLE 2
Further SNPS significantly associated
with HSA, LSA, or both LSA and HSA

| | | Position | Alleles |
|---|---|---|---|
| SNP ID | Chr | (bp) | (risk/non-risk) |
| BICF2G630183354 | 5 | 36,417,176 | C/T |
| BICF2G630183623 | 5 | 36,839,546 | T/C |
| BICF2G630183652 | 5 | 36,882,261 | A/G |
| BICF2P885817 | 19 | 35,831,635 | T/A |
| BICF2G63035476 | 5 | 32,708,612 | G/A |
| BICF2S23317145 | 5 | 32,725,862 | G/A |
| BICF2P1405079 | 5 | 32,757,545 | T/G |
| BICF2G63035510 | 5 | 32,757,807 | C/A |
| BICF2G63035542 | 5 | 32,771,537 | C/T |
| BICF2G63035564 | 5 | 32,787,898 | A/G |
| BICF2G63035577 | 5 | 32,804,686 | C/T |
| BICF2G63035700 | 5 | 32,876,294 | G/T |
| BICF2G63035705 | 5 | 32,879,166 | G/T |
| BICF2G63035726 | 5 | 32,901,346 | C/T |
| BICF2G63035729 | 5 | 32,902,463 | C/T |
| BICF2G630183626 | 5 | 36,845,402 | C/T |
| BICF2G630183630 | 5 | 36,848,237 | C/T |
| BICF2G630183805 | 5 | 37,081,986 | A/G |
| BICF2P267306 | 5 | 37,099,612 | A/G |
| BICF2P1337948 | 5 | 37,111,219 | G/T |
| BICF2P125723 | 5 | 56,109,903 | T/G |
| BICF2S23516471 | 11 | 36,928,683 | T/C |
| BICF2P22260 | 11 | 40,632,694 | A/T |
| BICF2P858820 | 11 | 40,794,422 | T/C |
| BICF2P260258 | 11 | 41,830,089 | T/C |
| BICF2P1020079 | 11 | 41,864,823 | C/T |
| BICF2P1362415 | 13 | 64,457,753 | T/C |
| BICF2G630746301 | 13 | 64,471,848 | C/T |
| BICF2P354069 | 18 | 52,718,637 | T/C |
| BICF2G630105651 | 25 | 47,612,990 | T/C |
| BICF2S23634252 | 33 | 25,953,846 | A/C |
| BICF2S23035109 | 5 | 11,757,453 | G/A |
| BICF2G63035383 | 5 | 32,622,509 | T/A |
| BICF2G63035403 | 5 | 32,632,285 | T/C |
| BICF2G63035476 | 5 | 32,708,612 | G/A |
| BICF2S23317145 | 5 | 32,725,862 | G/A |
| BICF2P1405079 | 5 | 32,757,545 | T/G |
| BICF2G63035510 | 5 | 32,757,807 | C/A |
| BICF2G63035542 | 5 | 32,771,537 | C/T |
| BICF2G63035564 | 5 | 32,787,898 | A/G |
| BICF2G63035577 | 5 | 32,804,686 | C/T |
| BICF2G63035700 | 5 | 32,876,294 | G/T |
| BICF2G63035705 | 5 | 32,879,166 | G/T |
| BICF2G63035726 | 5 | 32,901,346 | C/T |
| BICF2G63035729 | 5 | 32,902,463 | C/T |
| BICF2P93507 | 5 | 33,009,401 | A/G |
| BICF2G630183626 | 5 | 36,845,402 | C/T |
| BICF2G630183630 | 5 | 36,848,237 | C/T |
| BICF2G630183805 | 5 | 37,081,986 | A/G |
| BICF2P267306 | 5 | 37,099,612 | A/G |
| BICF2P1337948 | 5 | 37,111,219 | G/T |
| BICF2P858820 | 11 | 40,794,422 | T/C |

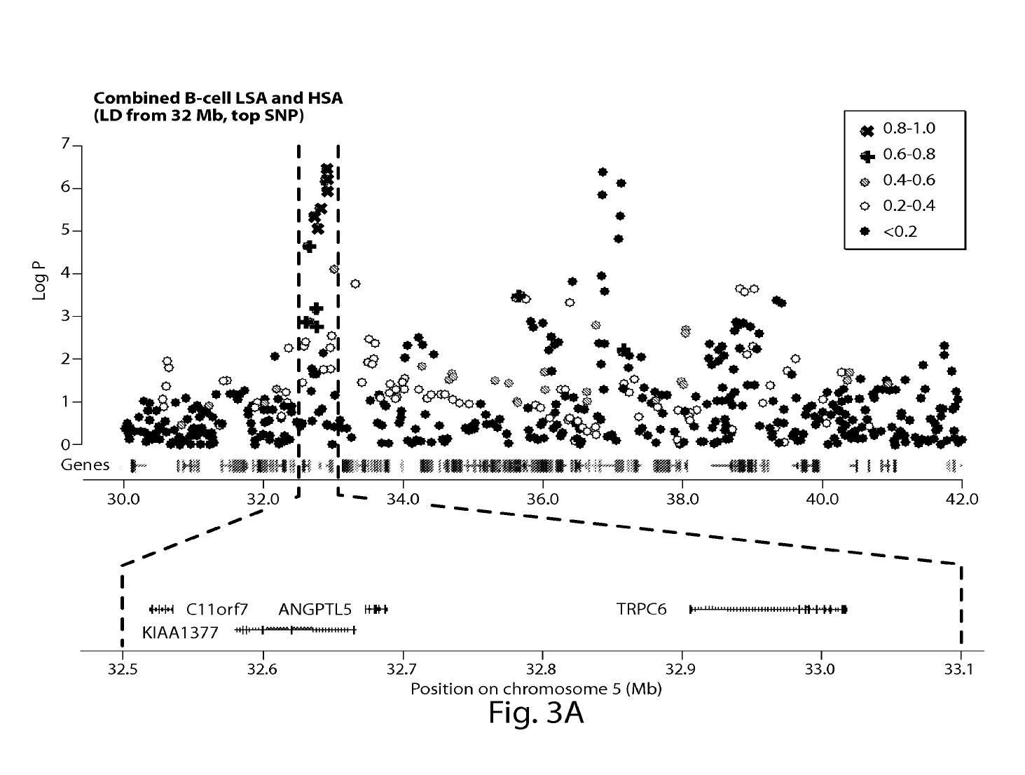

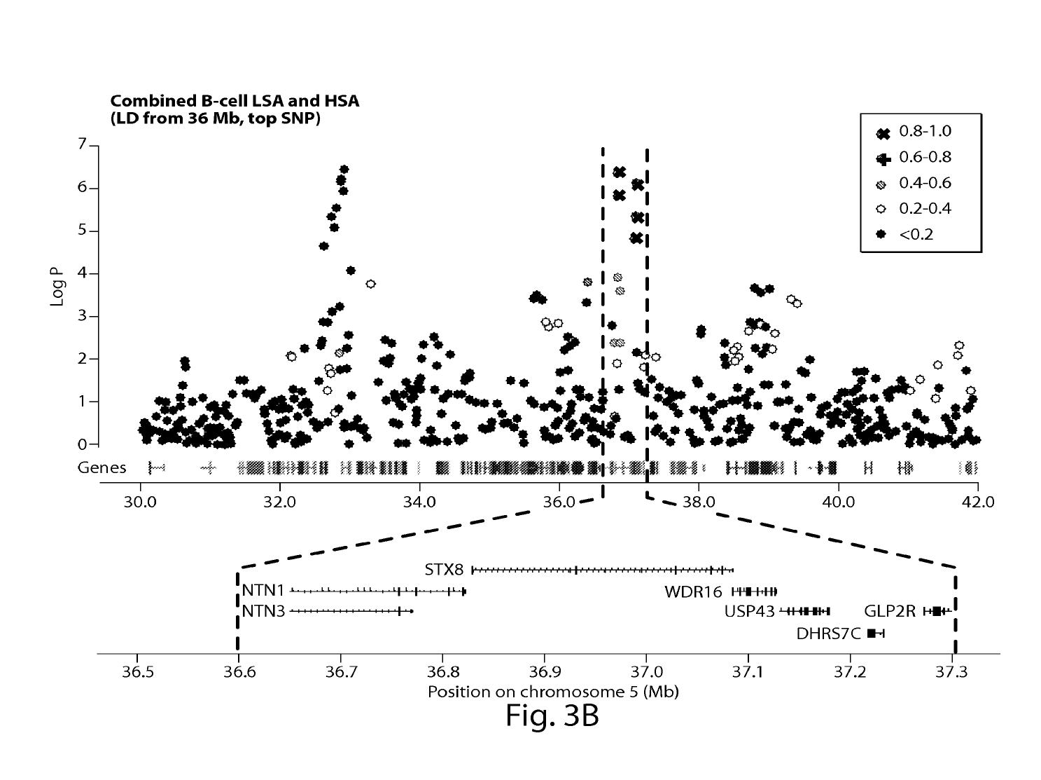

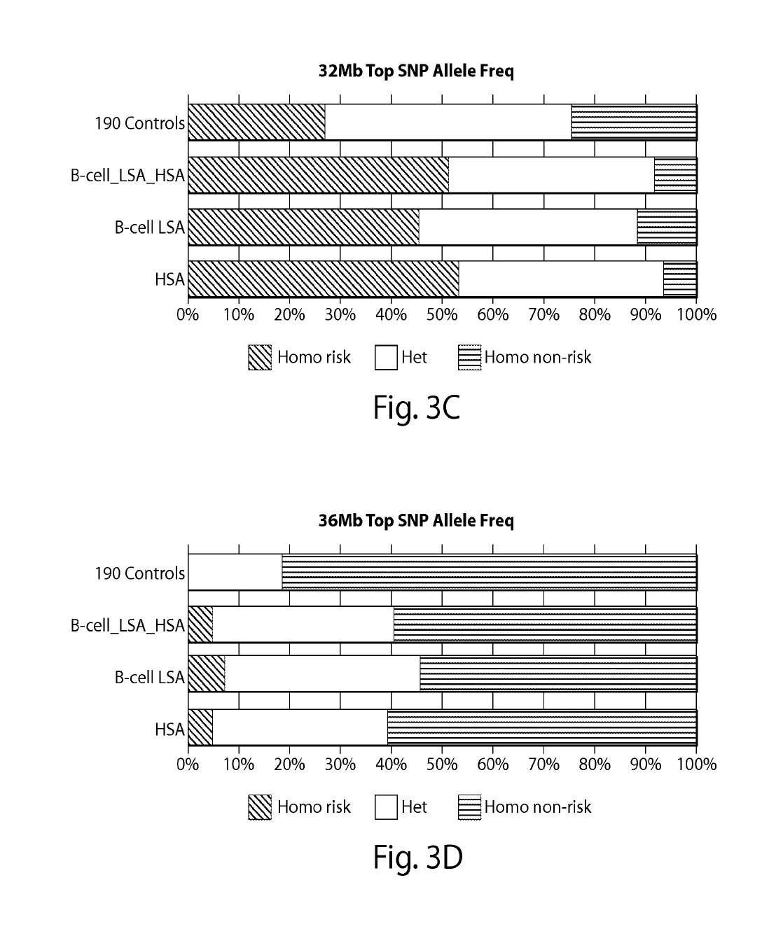

A more in-depth analysis revealed the presence of two linkage disequilibrium (LD) regions on chromosome 5 that were independently disproportionately represented in the subjects having LSA and HSA compared to the control subjects. The first region spanned an area on chromosome 5 from about 32.5 Mb to about 33.1 Mb. This region was identified according to the SNP BICF2G63035726. It is also identified as position 32,901,346 bp, CamFam2.0. This region may also be identified using one or more of the SNPs in Table 2 located within the boundaries of the first region. The second region spanned an area on chromosome 5 from about 36.6 Mb to about 37.3 Mb. This region was identified according to the SNP BICF2G630183630. It is also identified as position 36,848,237 bp, CamFam2.0. This region may also be identified using one or more of the SNPs in Table 2 located within the boundaries of the second region. Details relating to these two chromosome 5 LD regions are shown in Table 4 in the Examples section. Schematics of these chromosome 5 regions are provided in FIGS. 3A and 3B. Germ-line alleles, markers and mutations refer to alleles, markers and mutations that exist in all cells of an organism since they were present in the gametes that combined to form the organism. In contrast, somatic alleles, markers and mutations refer to alleles, markers and mutations that exist in a subset of cells are usually the result of mutation during the life span of the organism.

### Chromosome 5 Germ-Line Markers

The chromosome 5 risk-associated regions comprise a number of loci that may be the downstream mediators of the elevated cancer risk phenotype. FIGS. 3A and 3B shows the position of various of these loci in the two chromosome 5 regions. These loci include C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1 (and generating transcripts comprising SEQ ID NO:2).

The locus comprising the nucleotide sequence of SEQ ID NO:1 is a novel locus. The sequence is provided in Table 8. Its coordinates, on CamFam2.0 genome, are chr5:32732962-32766974. The underlined and bolded sequences correspond to a novel transcript made by the locus.

In accordance with the invention, all of these loci were sequenced in order to identify particular mutations that may be associated with elevated cancer risk. These sequencing studies identified a number of loci that are mutated in tumors carrying one or both germ-line risk alleles. Exemplary mutations found within the coding sequence include those in the following loci: KIAA1377, ANGPTL5 and TRP6. Details relating to these mutations are provided in Table 5 in the Examples section. As indicated in the Table, germ-line mutations were detected in these loci but somatic mutations (as described below) were not.

The invention contemplates methods that sequence these chromosome 5 specific markers and identify subjects having mutations in these markers. The presence of such mutations is associated with an elevated risk of developing cancer or the presence of an otherwise undetectable cancer, according to the invention. The invention further contemplates that mutations in these markers may exist in their regulatory and/or coding regions. As a result, sequencing analysis may be performed on mRNA transcripts or cDNA counterparts (for coding region mutations) or on genomic DNA (for regulatory region mutations). As used herein, regulatory regions are those nucleotide sequences (and regions) that control the temporal and/or spatial expression of a gene but typically do not contribute to the amino acid sequence of their gene product. As used herein, coding regions are those nucleotide sequences (and regions) that dictate the amino acid sequence of the encoded gene product. Methods for sequencing such markers are described herein.

### Differentially Expressed Chromosome 5 Germ-Line Markers

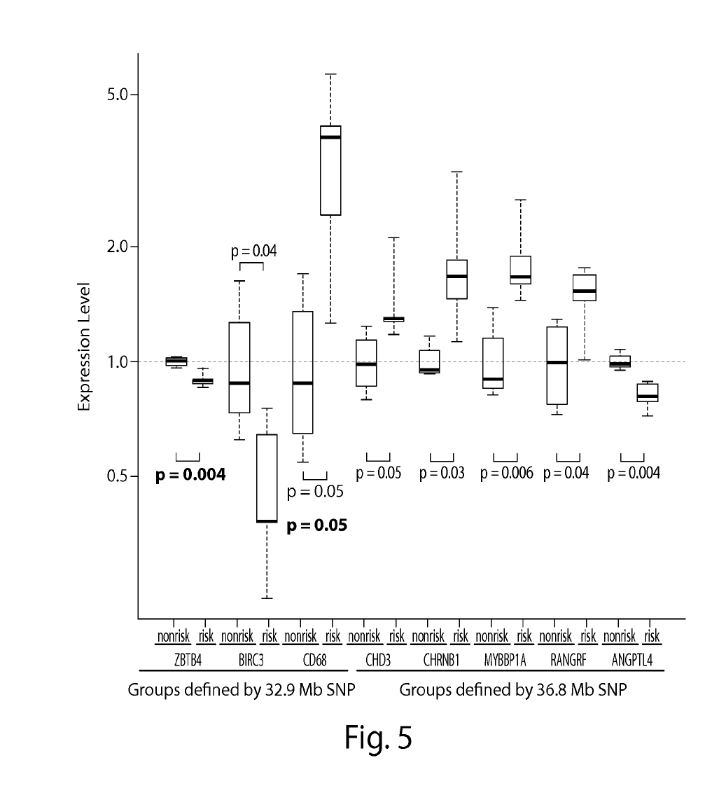

An analysis of the expression levels in tumors carrying one or both of the germ-line risk-associated alleles as compared to expression levels in tumors that did not carry the risk-associated allele(s) revealed differential expression of some of the chromosome 5 germ-line markers. Some of the markers, including ANGPTL5, BIRC3, CD68, MYBBP1A, CHD3, CHRNB1, RANGRF, ZBTB4, and a locus comprising SEQ ID NO:1, were differentially expressed in the tumors carrying the germ-line risk-associated allele(s) compared to the tumors that did not contain the germ-line risk-associated allele(s). More specifically, it was further found that certain markers were down-regulated in tumors carrying the germ-line allele(s) while others were up-regulated in tumors carrying the germ-line allele(s) compared to a tumor that does not carry the germ-line allele(s). The markers that are down-regulated include ZBTB4, BIRC3 and ANGPTL5. The markers that are up-regulated include CD68, CHD3, CHRNB1, MYBBP1A and RANGRF. The tumors therefore could be characterized at the molecular level based on the expression profile or one or more of these markers. The expression profile composites from the analysis of LSA and HSA tumors are provided in FIG. 5.

Additionally, other markers on chromosome 5, including TRPC6, KIAA1377, PIK3R6, ANGPTL5, HS3ST3B1, and BIRC3, were differentially expressed in the tumors carrying the germ-line risk-associated allele(s) compared to the tumors that did not contain the germ-line risk-associated allele(s). TRPC6, KIAA1377, PIK3R6, ANGPTL5 and BIRC3 were found to be down-regulated in tumors carrying the germ-line allele(s) compared to a tumor that does not carry the germ-line allele(s). HS3ST3B1 was found to be up-regulated in tumors carrying the germ-line allele(s) compared to a tumor that does not carry the germ-line allele(s). In some embodiments, the chromosome 5 marker is TRPC6.

Additionally, other markers on chromosome 5, including XLOC_{—}083025, PLEKHG5, TMPRSS13, TNFRSF18, and TNFRSF4, were differentially expressed in the tumors carrying the germ-line risk-associated allele(s) compared to the tumors that did not contain the germ-line risk-associated allele(s). TMPRSS13, TNFRSF18, and TNFRSF4 were found to be down-regulated in tumors carrying the germ-line allele(s) compared to a tumor that does not carry the germ-line allele(s). XLOC_{—}083025 and PLEKHG5 were found to be up-regulated in tumors carrying the germ-line allele(s) compared to a tumor that does not carry the germ-line allele(s).

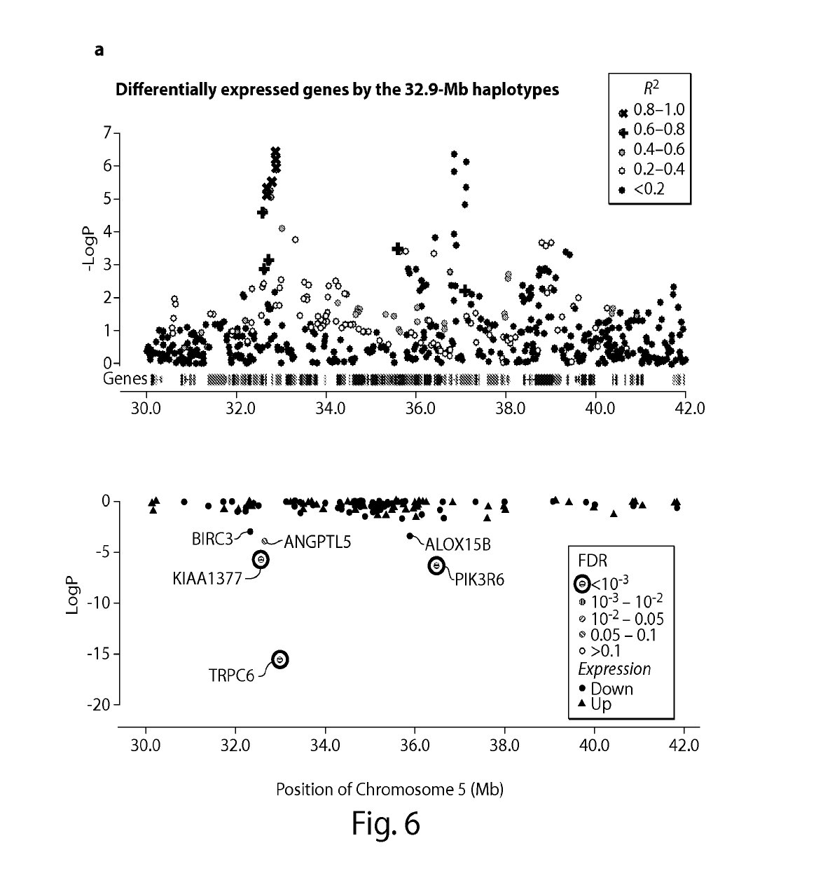

Expression data related to these markers are provided in FIG. 6 and Tables 12 and 13.

Accordingly, in view of these findings, the invention contemplates methods for measuring the level of expression of one or more of these markers and then identifying a subject that is at elevated risk of developing cancer or that has an as yet undiagnosed cancer based on an expression level profile similar to that provided herein.

The differential expression of various of the chromosome 5 markers suggests that mutations in these markers may occur in the regulatory region instead of or in addition to the coding region. A marker that appears to have mutations in both regulatory and coding regions is the ANGPTL5 gene.

### Other Differentially Expressed Markers

The invention is further premised in part on the discovery that other non-chromosome 5 genes are differentially expressed in tumors carrying the germ-line risk-associated allele(s) and tumors that do not carry the germ-line risk-associated allele(s). These non-chromosome 5 genes are as follows: ABTB1, AGA, AK1, ANXA1, B4GALT3, BAG3, BAT1, BCAT2, BEX4, BID, BIRC3, BTBD9, CCDC134, CCDC18, CCDC88C, CD1C, CD320, CD68, CDKN1A, CMTM8, COASY, COL7A1, CPT1B, CTSD, DDX41, DENND4B, DGKA, DHRS1, DUSP6, ECM1, EFCAB3, EIF4B, LOC478066, FABP3, FADS1, FBXL6, FBXO11, FBXO33, FBXW7, FNBP4, GALNT6, GBE1, GDPD3, GNGT2, GPR137B, GSTM1, GTF2IRD2, GTF3C3, GUCY1B3, HBD, LOC609402, ICAM4, IRF5, KIF5C, KLHDC1, KLHDC9, LBX2, LOC475952, LOC479273, LOC479683, LOC482085, LOC482088, LOC482361, LOC482532, LOC482790, LOC483843, LOC484249, LOC484784, LOC485196, LOC487557, LOC487994, LOC490377, LOC490693, LOC491116, LOC609521, LOC610353, LOC610841, LOC611771, LOC612387, LOC612917, LOXL3, LZTS2, MED24, MFSD6, MTA3, MYC, MYO19, NAGA, NAPRT1, NEIL1, NQO2, OGT, OSGEPL1, OVGP1, P2RX5, PDLIM7, PER3, PHKA2, PIGV, PIK3R6, PITPNM1, PRKRA, PVRIG, RAB24, RAB25, RABEP2, RASAL1, RBM11, RBM18, RBM35A, RBPJ, REC8, RILPL1, RPA1, SIGLEC12, SLC37A1, SP1, SUOX, TIMM22, TIPARP, TLE4, TMED6, TMEM41B, TNFSF8, TRAF5, TRMT1, TTC39C, TTF1, TUBB2A, UNC93B1, YWHAE, ZFAND2A, ZFC3H1, ZMAT1, ZMYM1, ZNF215, ZNF292, ZNF331, ZNF513, ZNF608, ZNF674, and ZNF711.

The markers that are up-regulated compared to control are as follows: ANXA1, BCAT2, BEX4, BID, BTBD9, CCDC18, CD1C, CD320, CD68, COASY, CTSD, DDX41, EFCAB3, FABP3, FBXL6, FBXO11, FBXO33, FBXW7, FNBP4, GBE1, GTF3C3, GUCY1B3, ICAM4, KLHDC1, LOC484249, LOC485196, LOC487557, LOC487994, LOC491116, LOC609521, LOC610353, LOC610841, LOC611771, LOC612917, LOXL3, MED24, MFSD6, MTA3, MYC, MYO19, NQO2, OGT, OSGEPL1, OVGP1, PDLIM7, PER3, PIGV, PITPNM1, PRKRA, RAB24, RASAL1, RBM35A, RILPL1, SIGLEC12, SLC37A1, SP1, SUOX, TIPARP, TMEM41B, TRMT1, TTF1, UNC93B1, ZFAND2A, ZFC3H1, ZNF215, ZNF331, ZNF608, ZNF674, and ZNF711.

The remaining markers in the list are down-regulated compared to the control.

Further non-chromosome 5 genes are as follows: C1GALT1, FGFR4, SCARA5, GFRA2, CD5L, CXL10, SLC25A48, KRT24, RP11-10N16.3, RPL6, ENSCAFG00000029323, XLOC_{—}011971, ENSCAFG00000013622, XLOC_{—}102336, and HIST1H. C1GALT1, FGFR4, SCARA5, GFRA2, CD5L, CXL10, SLC25A48, KRT24, and RP11-10N16.3 were found to be down-regulated in tumors carrying the germ-line allele(s) compared to a tumor that does not carry the germ-line allele(s). RPL6, ENSCAFG00000029323, XLOC_{—}011971, ENSCAFG00000013622, XLOC_{—}102336, and HIST1H were found to be up-regulated in tumors carrying the germ-line allele(s) compared to a tumor that does not carry the germ-line allele(s).

Further non-chromosome 5 genes are as follows: C1GALT1, EXTL1, B6F250, ENSCAFG00000030890, XLOC_{—}088759, RGS13, KRT24, RP11-10N16.3, TNFAIP3, CD8A, Q95J95, XLOC_{—}094643, SLC25A48, FGFR4, GPC3, NKG7, CXCR3, CD5L, PADI4, CXL10, GFRA2, SLC38A11, FABP4, PTPN22, ENSCAFG00000028509, U6, XLOC_{—}044225, XLOC_{—}100547, CCL22, CCDC168, TNIK, ENSCAFG00000030894, RGS10, HTR4, CNNM1, FBXO11, GRM5, SCARA5, OBSL1, RAB19, GZMK, ENSCAFG00000031494, TRBC2, TNFRSF21, ENSCAFG00000031437, GZMB, XLOC_{—}091705, CHGA, H6BA90, GALNT13, CACNA1D, CD8B, XLOC_{—}024761, EOMES, ZNF662, AFF2, COL6A6, HTRA1, LAD1, ENSCAFG00000029236, SCN2A, XLOC_{—}077615, KIAA1456, CCL19, KIF5C, XLOC_{—}026187, GPR27, ENSCAFG00000028940, ADAMTS2, CCL5, MAPK11, SMOC1, ABCA4, KIAA1598, KLRK1, LAT, FAM190A, ENSCAFG00000029467, PGBD5, TBXA2R, CSF1, MT1, ENSCAFG00000029651, RPL6, CHRM4, CD300A, KEL, RP11-664D7.4, MARCKSL1, TCTEX1D4, PROK2, LBH, NPDC1, CCR6, XLOC_{—}022131, ENSCAFG00000028850, XLOC_{—}068212, HIST1H, DLGAP3, MPO, CD151, XLOC_{—}067564, NETO1, U2, XLOC_{—}011971, GZMA, ENSCAFG00000013622, ENSCAFG00000029323, ENSCAFG00000029323, and XLOC_{—}102336.

The markers that are up-regulated compared to control are as follows:

CSF1, MT1, ENSCAFG00000029651, RPL6, CHRM4, CD300A, KEL, RP11-664D7.4, MARCKSL1, TCTEX1D4, PROK2, LBH, NPDC1, CCR6, XLOC_{—}022131, ENSCAFG00000028850, XLOC_{—}068212, HIST1H, DLGAP3, MPO, CD151, XLOC_{—}067564, NETO1, U2, XLOC_{—}011971, GZMA, ENSCAFG00000013622, ENSCAFG00000029323, ENSCAFG00000029323, and XLOC_{—}102336.

The markers that are down-regulated compared to control are as follows:

C1GALT1, EXTL1, B6F250, ENSCAFG00000030890, XLOC_{—}088759, RGS13, KRT24, RP11-10N16.3, TNFAIP3, CD8A, Q95J95, XLOC_{—}094643, SLC25A48, FGFR4, GPC3, NKG7, CXCR3, CD5L, PADI4, CXL10, GFRA2, SLC38A11, FABP4, PTPN22, ENSCAFG00000028509, U6, XLOC_{—}044225, XLOC_{—}100547, CCL22, CCDC168, TNIK, ENSCAFG00000030894, RGS10, HTR4, CNNM1, FBXO11, GRM5, SCARA5, OBSL1, RAB19, GZMK, ENSCAFG00000031494, TRBC2, TNFRSF21, ENSCAFG00000031437, GZMB, XLOC_{—}091705, CHGA, H6BA90, GALNT13, CACNA1D, CD8B, XLOC_{—}024761, EOMES, ZNF662, AFF2, COL6A6, HTRA1, LAD1, ENSCAFG00000029236, SCN2A, XLOC_{—}077615, KIAA1456, CCL19, KIF5C, XLOC_{—}026187, GPR27, ENSCAFG00000028940, ADAMTS2, CCL5, MAPK11, SMOC1, ABCA4, KIAA1598, KLRK1, LAT, FAM190A, ENSCAFG00000029467, PGBD5, and TBXA2R.

Expression data related to these markers are provided in FIG. 6 and Tables 12 and 13.

The invention therefore contemplates methods for identifying subjects at elevated risk of developing cancer based on aberrant expression levels of one or more of these genes compared to a control.

In some embodiments, the invention contemplates detection and/or use of chromosome 5 genes and non-chromosome 5 genes that are differentially expressed in tumors carrying the germ-line risk-associated allele(s) and tumors that do not carry the germ-line risk-associated allele(s). In some embodiments, the chromosome 5 genes and non-chromosome 5 genes are selected from TRPC6, C1GALT1, RPL6, PIK3R6, ENSCAFG00000029323, XLOC_{—}011971, FGFR4, SCARA5, GFRA2, KIAA1377, ENSCAFG00000013622, CD5L, XLOC_{—}102336, CXL10, SLC25A48, KRT24, ENSCAFG00000029323, RP11-10N16.3, HIST1H, or HS3ST3B1.

TRPC6, C1GALT1, PIK3R6, FGFR4, SCARA5, GFRA2, KIAA1377, CD5L, CXL10, SLC25A48, KRT24, and RP11-10N16.3 were found to be down-regulated in tumors carrying the germ-line allele(s) compared to a tumor that does not carry the germ-line allele(s).

RPL6, ENSCAFG00000029323, XLOC_{—}011971, ENSCAFG00000013622, XLOC_{—}102336, ENSCAFG00000029323, HIST1H, and HS3ST3B1 were found to be up-regulated in tumors carrying the germ-line allele(s) compared to a tumor that does not carry the germ-line allele(s). Expression data related to these markers are provided in FIG. 6 and Table 12.

### Somatic Markers

The invention is also based in part on the discovery of various somatic mutations present in tumors carrying the germ-line allele(s) as compared to tumors that do not carry the germ-line allele(s). Somatic mutations were identified by performing a genome-wide sequencing of tumor cells and normal cells from dogs with LSA. The markers demonstrating a mutation are TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1.

The invention therefore provides methods for detecting the presence of a mutation in one or more of these genes and identifying a subject at elevated risk of developing cancer or having an as yet undiagnosed cancer based on the presence of such mutation(s).

In some instances, the invention provides methods for detecting the presence of a mutation in one or more of ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, and identifying a subject at elevated risk of developing cancer based on the presence of such mutation(s). The subject may be a canine subject or a human subject, although it is not so limited.

Table 3 lists the NCBI database accession numbers for several of these markers in the canine genome and in the human genome. In some instances, a human orthologue of the locus has not yet been identified. In those instances, the invention contemplates that the human orthologue possesses at least 60% homology, or at least 70% homology, or at least 75% homology to the canine sequence and the methods described herein can be based on an analysis of loci in the human genome that share these degrees of homology.

### Genome Analysis Methods

Methods of genetic analysis are known in the art. Examples of genetic analysis methods and commercially available tools are described below.

Affymetrix:

The Affymetrix SNP 6.0 array contains over 1.8 million SNP and copy number probes on a single array. The method utilizes at a simple restriction enzyme digestion of 250 ng of genomic DNA, followed by linker-ligation of a common adaptor sequence to every fragment, a tactic that allows multiple loci to be amplified using a single primer complementary to this adaptor. Standard PCR then amplifies a predictable size range of fragments, which converts the genomic DNA into a sample of reduced complexity as well as increases the concentration of the fragments that reside within this predicted size range. The target is fragmented, labeled with biotin, hybridized to microarrays, stained with streptavidin-phycoerythrin and scanned. To support this method, Affymetrix Fluidics Stations and integrated GS-3000 Scanners can be used.

Illumina Infinium:

Examples of commercially available Infinium array options include the 660W-Quad (>660,000 probes), the 1 MDuo (over 1 million probes), and the custom iSelect (up to 200,000 SNPs selected by user). Samples begin the process with a whole genome amplification step, then 200 ng is transferred to a plate to be denatured and neutralized, and finally plates are incubated overnight to amplify. After amplification the samples are enzymatically fragmented using end-point fragmentation. Precipitation and resuspension clean up the DNA before hybridization onto the chips. The fragmented, resuspended DNA samples are then dispensed onto the appropriate BeadChips and placed in the hybridization oven to incubate overnight. After hybridization the chips are washed and labeled nucleotides are added to extend the primers by one base. The chips are immediately stained and coated for protection before scanning. Scanning is done with one of the two Illumina iScan™ Readers, which use a laser to excite the fluorophore of the single-base extension product on the beads. The scanner records high-resolution images of the light emitted from the fluorophores. All plates and chips are barcoded and tracked with an internally derived laboratory information management system. The data from these images are analyzed to determine SNP genotypes using Illumina's BeadStudio. To support this process, Biomek F/X, three Tecan Freedom Evos, and two Tecan Genesis Workstation 150s can be used to automate all liquid handling steps throughout the sample and chip prep process.

Illumina BeadArray:

The Illumina Bead Lab system is a multiplexed array-based format. Illumina's BeadArray Technology is based on 3-micron silica beads that self-assemble in microwells on either of two substrates: fiber optic bundles or planar silica slides. When randomly assembled on one of these two substrates, the beads have a uniform spacing of ˜5.7 microns. Each bead is covered with hundreds of thousands of copies of a specific oligonucleotide that act as the capture sequences in one of Illumina's assays. BeadArray technology is utilized in Illumina's iScan System.

Sequenom:

During pre-PCR, either of two Packard Multiprobes is used to pool oligonucleotides, and a Tomtec Quadra 384 is used to transfer DNA. A Cartesian nanodispenser is used for small-volume transfer in pre-PCR, and another in post-PCR. Beckman Multimeks, equipped with either a 96-tip head or a 384-tip head, are used for more substantial liquid handling of mixes. Two Sequenom pin-tool are used to dispense nanoliter volumes of analytes onto target chips for detection by mass spectrometry. Sequenom Compact mass spectrometers can be used for genotype detection.

### Sequencing Methods

Methods of genome sequencing are known in the art. Examples of genome sequencing methods and commercially available tools are described below.

Illumina Sequencing:

89 GAIIx Sequencers are used for sequencing of samples. Library construction is supported with 6 Agilent Bravo plate-based automation, Stratagene MX3005p qPCR machines, Matrix 2-D barcode scanners on all automation decks and 2 Multimek Automated Pipettors for library normalization.

454 Sequencing:

Roche® 454 FLX-Titanium instruments are used for sequencing of samples. Library construction capacity is supported by Agilent Bravo automation deck, Biomek FX and Janus PCR normalization.

SOLiD Sequencing:

SOLiD v3.0 instruments are used for sequencing of samples. Sequencing set-up is supported by a Stratagene MX3005p qPCR machine and a Beckman SC Quanter for bead counting.

ABI Prism® 3730 XL Sequencing:

ABI Prism® 3730 XL machines are used for sequencing samples. Automated Sequencing reaction set-up is supported by 2 Multimek Automated Pipettors and 2 Deerac Fluidics—Equator systems. PCR is performed on 60 Thermo-Hybaid 384-well systems.

Ion Torrent:

Ion PGM™ or Ion Proton™ machines are used for sequencing samples. Ion library kits (Invitrogen) can be used to prepare samples for sequencing.

Other Technologies:

Examples of other commercially available platforms include Helicos Heliscope Single-Molecule Sequencer, Polonator G.007, and Raindance RDT 1000 Rainstorm.

### Expression Level Analysis

The invention contemplates that elevated risk of developing certain cancers is associated with an altered expression pattern of one or more genes some but not all of which are located on chromosome 5 at or near the germ-line risk-associated alleles identified by the invention. The invention therefore contemplates methods that involve measuring the mRNA or protein levels for these genes and comparing such levels to control levels, including for example predetermined thresholds.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays.

Expression profiles of cells in a biological sample (e.g., blood or a tumor) can be carried out using an oligonucleotide microarray analysis. As an example, this analysis may be carried out using a commercially available oligonucleotide microarray or a custom designed oligonucleotide microarray comprising oligonucleotides for all or a subset of the germ-line markers described herein. The microarray may comprise any number of the germ-line markers, as the invention contemplates that elevated risk may be determined based on the analysis of single differentially expressed markers or a combination of differentially expressed markers. The markers may be those that are up-regulated in tumors carrying a risk allele (compared to a tumor that does not carry the risk allele), or those that are down-regulated in tumors carrying a risk allele (compared to a tumor that does not carry the risk allele), or a combination of these. The number of markers measured using the microarray therefore may be 1, 2, 3, 4, 5, 6, 7, 8, 9, 10 or more markers selected from the group consisting of ANGPTL5, BIRC3, CD68, MYBBP1A, CHD3, CHRNB1, RANGRF, ZBTB4, and a locus comprising SEQ ID NO:1, and/or any other markers listed in Tables 12 and/or 13. It is to be understood that such arrays may however also comprise positive and/or negative control markers such as housekeeping genes that can be used to determine if the array has been degraded and/or if the sample has been degraded or contaminated. The art is familiar with the construction of oligonucleotide arrays.

Commercially available gene expression systems include Affymetrix GeneChip microarrays as well as all of Illumina standard expression arrays, including two GeneChip 450 Fluidics Stations and a GeneChip 3000 Scanner, Affymetrix High-Throughput Array (HTA) System composed of a GeneStation liquid handling robot and a GeneChip HT Scanner providing automated sample preparation, hybridization, and scanning for 96-well Affymetrix PEGarrays. These systems can be used in the cases of small or potentially degraded RNA samples. The invention also contemplates analyzing expression levels from fixed samples (as compared to freshly isolated samples). The fixed samples include formalin-fixed and/or paraffin-embedded samples. Such samples may be analyzed using the whole genome Illumina DASL assay. High-throughput gene expression profile analysis can also be achieved using bead-based solutions, such as Luminex systems.

Other mRNA detection and quantitation methods include multiplex detection assays known in the art, e.g., xMAP® bead capture and detection (Luminex Corp., Austin, Tex.).

Another exemplary method is a quantitative RT-PCR assay which may be carried out as follows: mRNA is extracted from cells in a biological sample (e.g., blood or a tumor) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. Probes may be designed using the sequences or sequence identifiers listed in Table 3 or using sequences associated with the provided Ensembl gene IDs. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Assays

The art is familiar with various methods for measuring protein levels. Protein levels may be measured using protein-based assays such as but not limited to immunoassays, Western blots, Western immunoblotting, multiplex bead-based assays, and assays involving aptamers (such as SOMAmer™ technology) and related affinity agents.

An exemplary immunoassay may be carried out as follows: A biological sample is applied to a substrate having bound to its surface marker-specific binding partners (i.e., immobilized marker-specific binding partners). The marker-specific binding partner (which may be referred to as a “capture ligand” because it functions to capture and immobilize the marker on the substrate) may be an antibody or an antigen-binding antibody fragment such as Fab, F(ab)2, Fv, single chain antibody, Fab and sFab fragment, F(ab′)₂, Fd fragments, scFv, and dAb fragments, although it is not so limited. Other binding partners are described herein. Markers present in the biological sample bind to the capture ligands, and the substrate is washed to remove unbound material. The substrate is then exposed to soluble marker-specific binding partners (which may be identical to the binding partners used to immobilize the marker). The soluble marker-specific binding partners are allowed to bind to their respective markers immobilized on the substrate, and then unbound material is washed away. The substrate is then exposed to a detectable binding partner of the soluble marker-specific binding partner. In one embodiment, the soluble marker-specific binding partner is an antibody having some or all of its Fc domain. Its detectable binding partner may be an anti-Fc domain antibody. As will be appreciated by those in the art, if more than one marker is being detected, the assay may be configured so that the soluble marker-specific binding partners are all antibodies of the same isotype. In this way, a single detectable binding partner, such as an antibody specific for the common isotype, may be used to bind to all of the soluble marker-specific binding partners bound to the substrate.

It is to be understood that the substrate may comprise capture ligands for one or more markers, including two or more, three or more, four or more, five or more, etc. up to and including all nine of the markers provided by the invention.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include marker-specific binding partners. Marker-specific binding partners may be designed using the sequences or sequence identifiers listed in Table 3 or using sequences associated with the provided Ensembl gene IDs. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target marker, e.g., through non-covalent bonding. For example, if the marker is a ligand, a binding partner may be a receptor for that ligand. In another example, if the marker is a receptor, a binding partner may be a ligand for that receptor. In yet another example, a binding partner may be a protein or peptide known to interact with a marker. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

Binding partners also include any molecule capable of demonstrating selective binding to any one of the target markers disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

### Detectable Labels

Detectable binding partners may be directly or indirectly detectable. A directly detectable binding partner may be labeled with a detectable label such as a fluorophore. An indirectly detectable binding partner may be labeled with a moiety that acts upon (e.g., an enzyme or a catalytic domain) or is acted upon (e.g., a substrate) by another moiety in order to generate a detectable signal. These various methods and moieties for detectable labeling are known in the art.

### Controls

Some of the methods provided herein involve measuring a level of a marker in a biological sample and then comparing that level to a control in order to identify a subject having an elevated risk of developing a cancer such as a hematological cancer. The control may be a control level that is a level of the same marker in a control tissue, control subject, or a population of control subjects.

The control may be (or may be derived from) a normal subject (or normal subjects). Normal subjects, as used herein, refer to subjects that are apparently healthy and show no tumor manifestation. The control population may therefore be a population of normal subjects.

In other instances, the control may be (or may be derived from) a subject (a) having a similar tumor to that of the subject being tested and (b) who is negative for the germ-line risk allele.

It is to be understood that the methods provided herein do not require that a control level be measured every time a subject is tested. Rather, it is contemplated that control levels of markers are obtained and recorded and that any test level is compared to such a predetermined level (or threshold).

### Samples

The methods provided herein detect and sometimes measure (and thus analyze) levels or particular markers in biological samples. Biological samples, as used herein, refer to samples taken or derived from a subject. These samples may be tissue samples or they may be fluid samples (e.g., bodily fluid). Examples of biological fluid samples are whole blood, plasma, serum, urine, sputum, phlegm, saliva, tears, and other bodily fluids. In some embodiments, the biological sample is a whole blood sample, or a sample of white blood cells from a subject. In some embodiments, the biological sample is a tumor, a fragment of a tumor, or a tumor cell(s). The sample may be taken from the mouth of a subject using a swab or it may be obtained from other mucosal tissue in the subject.

### Subjects

Certain methods of the invention are intended for canine subjects, including for example golden retrievers. Other methods of the invention may be used in a variety of subjects including but not limited to humans and canine subjects.

### Computational Analysis

Methods of computation analysis of genomic and expression data are known in the art. Examples of available computational programs are: Genome Analysis Toolkit (GATK, Broad Institute, Cambridge, Mass.), Expressionist Refiner module (Genedata AG, Basel, Switzerland), GeneChip-Robust Multichip Averaging (CG-RMA) algorithm, PLINK (Purcell et al, 2007), GCTA (Yang et al, 2011), the EIGENSTRAT method (Price et al 2006), EMMAX (Kang et al, 2010).

### Breeding Programs

Other aspects of the invention relate to use of the diagnostic methods in connection with a breeding program. A breeding program is a planned, intentional breeding of a group of animals to reduce detrimental or undesirable traits and/or increase beneficial or desirable traits in offspring of the animals. Thus, a subject identified using the methods described herein as not having a risk marker of the invention may be included in a breeding program to reduce the risk of developing hematological cancer in the offspring of said subject. Alternatively, a subject identified using the methods described herein as having a risk marker of the invention may be excluded from a breeding program. In some embodiments, methods of the invention comprise exclusion of a subject identified as being at elevated risk of developing hematological cancer or having undiagnosed hematological cancer in a breeding program or inclusion of a subject identified as not being at elevated risk of developing hematological cancer or having undiagnosed hematological cancer in a breeding program.

### Treatment

Other aspects of the invention relate to diagnostic or prognostic methods that comprise a treatment step (also referred to as “theranostic” methods due to the inclusion of the treatment step). Any treatment for a hematological cancer, such as LSA or HSA, is contemplated herein. In some embodiments, treatment comprises one or more of surgery, chemotherapy, and radiation. Examples of chemotherapy for treatment of hematological cancers include rituximab, cyclophosphamide, doxorubicin, vincristine, and/or prednisone.

In some embodiments, a subject identified as being at elevated risk of developing hematological cancer or having undiagnosed hematological cancer is treated. In some embodiments, the method comprises selecting a subject for treatment on the basis of the presence of one or more risk markers as described herein. In some embodiments, the method comprises treating a subject with a hematological cancer characterized by the presence of one or more risk markers as defined herein.

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin). Dosage will depend on the subject and the route of administration. Dosage can be determined by the skilled artisan.

### Isolated Nucleic Acid Molecules

According to one aspect of the invention, isolated nucleic acid molecules are provided selected from the group consisting of: (a) nucleic acid molecules which hybridize under stringent conditions to a molecule consisting of a nucleic acid of SEQ ID NO: 1 or SEQ ID NO: 2, (b) deletions, additions and substitutions of (a), (c) nucleic acid molecules that differ from the nucleic acid molecules of (a) or (b) in codon sequence due to the degeneracy of the genetic code, and (d) complements of (a), (b) or (c). In some embodiments, the isolated nucleic acid molecule comprises SEQ ID NO: 1 or SEQ ID NO: 2. In some embodiments, the isolated nucleic acid molecule comprises SEQ ID NO:2.

The invention in another aspect provides an isolated nucleic acid molecule selected from the group consisting of (a) a unique fragment of nucleic acid molecule of SEQ ID NO:1 or SEQ ID NO: 2 (of sufficient length to represent a sequence unique within the canine genome) and (b) complements of (a).

In one embodiment, the sequence of contiguous nucleotides is selected from the group consisting of (1) at least two contiguous nucleotides nonidentical to the sequence group, (2) at least three contiguous nucleotides nonidentical to the sequence group, (3) at least four contiguous nucleotides nonidentical to the sequence group, (4) at least five contiguous nucleotides nonidentical to the sequence group, (5) at least six contiguous nucleotides nonidentical to the sequence group, (6) at least seven contiguous nucleotides nonidentical to the sequence group.

In another embodiment, the fragment has a size selected from the group consisting of at least: 8 nucleotides, 10 nucleotides, 12 nucleotides, 14 nucleotides, 16 nucleotides, 18 nucleotides, 20, nucleotides, 22 nucleotides, 24 nucleotides, 26 nucleotides, 28 nucleotides, 30 nucleotides, 40 nucleotides, 50 nucleotides, 75 nucleotides, 100 nucleotides, 200 nucleotides, 1000 nucleotides and every integer length there between.

According to another aspect, the invention provides expression vectors, and host cells transformed or transfected with such expression vectors, comprising the nucleic acid molecules described above.

Table 3 provides a list of the germ-line and somatic markers associated with elevated risk of tumors in canines. The canine Ensembl gene identifiers are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The Ensembl gene ID provided for each gene can be used to determine the nucleotide sequence of the gene, as well as associated transcript and protein sequences, by inputting the Ensemble ID into the Ensemble database (Ensembl release 70).

TABLE 3
List of germ-line and somatic markers associated with elevated risk of
tumors in canines

| | | Ensembl | Ensembl | | | |
|---|---|---|---|---|---|---|
| | Ensembl gene | transcript | protein ID(s), | | | |
| locus | ID, Canine | ID(s), Canine | Canine | Ensemble gene ID, Human | Ensemble transcript ID(s), Human | Ensembl protein ID(s), Human |
| BICF2G63035726 | | | | | | |
| BICF2G630183630 | | | | | | |
| C11orf7 | ENSCAFG00000009989 | | | ENSG00000174672 | | |
| ANGPTL5 | ENSCAFG00000023699 | ENSCAFT00000036595 | | ENSG00000187151 | ENST00000334289, | |
| | | | | | ENST00000534527 | |
| KIAA1377 | ENSCAFG00000015131 | | | ENSG00000110318 | | |
| TRPC6 | ENSCAFG00000015194 | | | ENSG00000137672 | | |
| NTN1 | ENSCAFG00000017403 | | | ENSG00000065320 | | |
| NTN3 | ENSCAFG00000019366 | ENSG00000162068 | | | | |
| STX8 | ENSCAFG00000017413 | ENSG00000170310 | | | | |
| WDR16 | ENSCAFG00000017427 | ENSG00000166596 | | | | |
| USP43 | ENSCAFG00000017439 | ENSG00000154914 | | | | |
| DHRS7C | ENSCAFG00000017443 | ENSG00000184544 | | | | |
| GLP2R | ENSCAFG00000017446 | ENSG00000065325 | | | | |
| BIRC3 | ENSCAFG00000015105 | ENSCAFT00000024001 | | ENSG00000023445 | ENST00000263464, | |
| | | | | | ENST00000527309, | |
| | | | | | ENST00000532609, | |
| | | | | | ENST00000532808 | |
| CD68 | ENSCAFG00000016641 | ENSCAFT00000026361, | | ENSG00000129226 | ENST00000250092, | |
| | | ENSCAFT00000036024 | | | ENST00000380498 | |
| MYBBP1A | ENSCAFG00000015266 | ENSCAFT00000024251 | | ENSG00000132382 | ENST00000381556, | |
| | | | | | ENST00000426435, | |
| | | | | | ENST00000254718 | |
| CHD3 | ENSCAFG00000016859 | ENSCAFT00000026729 | | ENSG00000170004 | ENST00000380358, | |
| | | | | | ENST00000330494, | |
| | | | | | ENST00000358181, | |
| | | | | | ENST00000439235, | |
| | | | | | ENST00000449744, | |
| | | | | | ENST00000452447 | |
| CHRNB1 | ENSCAFG00000016315 | ENSCAFT00000025890 | | ENSG00000170175 | ENST00000306071, | |
| | | | | | ENST00000536404 | |
| RANGRF | ENSCAFG00000017030 | ENSCAFT00000026971 | | ENSG00000108961 | ENST00000226105, | |
| | | | | | ENST00000407006, | |
| | | | | | ENST00000439238 | |
| ZBTB4 | ENSCAFG00000016341 | ENSCAFT00000025918 | | ENSG00000174282 | ENST00000380599, | |
| | | | | | ENST00000311403 | |
| TRAF3 | ENSCAFG00000018075 | ENSCAFT00000028725, | ENSCAFP00000026718, | ENSG00000131323 | ENST00000560463, | ENSP00000453623, |
| | | ENSCAFT00000028719 | ENSCAFP00000026713 | | ENST00000560371, | ENSP00000454207, |
| | | | | | ENST00000559734, | ENSP00000453032, |
| | | | | | ENST00000558880 | ENSP00000453031, |
| | | | | | (no protein | ENSP00000445998, |
| | | | | | product), | ENSP00000376500, |
| | | | | | ENST00000558700, | ENSP00000332468, |
| | | | | | ENST00000539721, | ENSP00000328003 |
| | | | | | ENST00000392745, | |
| | | | | | ENST00000351691, | |
| | | | | | ENST00000347662 | |
| FBXW7 | ENSCAFG00000008141 | ENSCAFT00000012962 | ENSCAFP00000011996 | ENSG00000109670 | ENST00000393956, | ENSP00000377528, |
| | | | | | ENST00000296555, | ENSP00000296555, |
| | | | | | ENST00000281708, | ENSP00000281708, |
| | | | | | ENST00000263981 | ENSP00000263981 |
| DOK6 | ENSCAFG00000000039 | ENSCAFT00000036237, | ENSCAFP00000031589, | ENSG00000206052 | ENST00000382713 | ENSP00000372160 |
| | | ENSCAFT00000000059 | ENSCAFP00000000052 | | | |
| RARS | ENSCAFG00000017058 | ENSCAFT00000027027 | ENSCAFP00000025127 | ENSG00000113643 | ENST00000538719, | ENSP00000439108, |
| | | | | | ENST00000524082 | ENSP00000430035, |
| | | | | | (NPP), | ENSP00000428494, |
| | | | | | ENST00000522834, | ENSP00000429030, |
| | | | | | ENST00000521939 | ENSP00000231572 |
| | | | | | (NPP), | |
| | | | | | ENST00000521329, | |
| | | | | | ENST00000520421 | |
| | | | | | (NPP), | |
| | | | | | ENST00000520013, | |
| | | | | | ENST00000519346 | |
| | | | | | (NPP), | |
| | | | | | ENST00000518757 | |
| | | | | | (NPP), | |
| | | | | | ENST00000231572 | |
| JPH3 | ENSCAFG00000019906 | ENSCAFT00000031672 | ENSCAFP00000029486 | ENSG00000154118 | ENST00000563609 | ENSP00000437801, |
| | | | | | (NPP), | ENSP00000301008, |
| | | | | | ENST00000537256, | ENSP00000284262 |
| | | | | | ENST00000301008, | |
| | | | | | ENST00000284262 | |
| LRRN3 | ENSCAFG00000003297 | ENSCAFT00000036387, | ENSCAFP00000031756, | ENSG00000173114 | ENST00000464835 | ENSP00000397312, |
| | | ENSCAFT00000005281 | ENSCAFP00000004886 | | (NPP), | ENSP00000412417, |
| | | | | | ENST00000451085, | ENSP00000407927, |
| | | | | | ENST00000422987, | ENSP00000312001 |
| | | | | | ENST00000421101, | |
| | | | | | ENST00000308478 | |
| MLL2 | ENSCAFG00000008718 | ENSCAFT00000013872 | ENSCAFP00000012833 | ENSG00000167548 | ENST00000552391 | ENSP00000449455, |
| | | | | | (NPP), | ENSP00000435714, |
| | | | | | ENST00000550356 | ENSP00000301067 |
| | | | | | (NPP), | |
| | | | | | ENST00000549799 | |
| | | | | | (NPP), | |
| | | | | | ENST00000549743 | |
| | | | | | (NPP), | |
| | | | | | ENST00000547610, | |
| | | | | | ENST00000526209, | |
| | | | | | ENST00000301067 | |
| OGT | ENSCAFG00000017134 | ENSCAFT00000027149 | ENSCAFP00000025249 | ENSG00000147162 | ENST00000498566 | ENSP00000407659, |
| | | | | | (NPP), | ENSP00000399729, |
| | | | | | ENST00000488174 | ENSP00000362824, |
| | | | | | (NPP), | ENSP00000362805 |
| | | | | | ENST00000474633 | |
| | | | | | (NPP), | |
| | | | | | ENST00000472270 | |
| | | | | | (NPP), | |
| | | | | | ENST00000466181 | |
| | | | | | (NPP), | |
| | | | | | ENST00000462638 | |
| | | | | | (NPP), | |
| | | | | | ENST00000459760 | |
| | | | | | (NPP), | |
| | | | | | ENST00000455587, | |
| | | | | | ENST00000444774, | |
| | | | | | ENST00000373719, | |
| | | | | | ENST00000373701 | |
| POU3F4 | ENSCAFG00000017368 | ENSCAFT00000027512 | ENSCAFP00000025584 | ENSG00000196767 | ENST00000373200 | ENSP00000362296 |
| SETD2 | ENSCAFG00000013392 | ENSCAFT00000021260 | ENSCAFP00000019740 | ENSG00000181555 | ENST00000543224, | ENSP00000438167, |
| | | | | | ENST00000492397 | ENSP00000389611, |
| | | | | | (NPP), | ENSP00000411901, |
| | | | | | ENST00000484689, | ENSP00000388349, |
| | | | | | (NPP), | ENSP00000416401, |
| | | | | | ENST00000479832 | ENSP00000386759, |
| | | | | | (NPP), | ENSP00000332415 |
| | | | | | ENST00000451092, | |
| | | | | | ENST00000445387, | |
| | | | | | ENST00000431180, | |
| | | | | | ENST00000412450, | |
| | | | | | ENST00000409792, | |
| | | | | | ENST00000330022 | |
| CACNA1G | ENSCAFG00000017120 | ENSCAFT00000027129 | ENSCAFP00000025229 | ENSG00000006283 | ENST00000515765 | ENSP00000426232 |
| | | | | | (33 other | |
| | | | | | variants, | |
| | | | | | coding and | |
| | | | | | non-coding) | |
| DSCAML1 | ENSCAFG00000012923 | ENSCAFT00000020576 | ENSCAFP00000019097 | ENSG00000177103 | ENST00000321322, | ENSP00000315465, |
| | | | | | ENST00000525836, | ENSP00000436387, |
| | | | | | ENST00000527706, | ENSP00000434335, |
| | | | | | ENST00000446508 | ENSP00000394795 |
| MLL | ENSCAFG00000012691 | ENSCAFT00000020182 | ENSCAFP00000018720 | ENSG00000118058 | ENST00000534358 | ENSP00000436786 |
| | | | | | (11 other | |
| | | | | | variants) | |
| ADD2 | ENSCAFG00000003407 | ENSCAFT00000005489 | ENSCAFP00000005087 | ENSG00000075340 | ENST00000264436 | ENSP00000264436 |
| | | | | | (11 other | |
| | | | | | variants) | |
| ARID1A | ENSCAFG00000012314 | ENSCAFT00000019631 | ENSCAFP00000018208 | ENSG00000117713 | ENST00000324856 | ENSP00000320485 |
| | | | | | (7 | |
| | | | | | other | |
| | | | | | variants) | |
| ARNT2 | ENSCAFG00000013922 | ENSCAFT00000022111 | ENSCAFP00000020531 | ENSG00000172379 | ENST00000303329 | ENSP00000307479 |
| | | | | | (2 | |
| | | | | | other | |
| | | | | | variants) | |
| CAPN12 | ENSCAFG00000005681 | ENSCAFT00000009152 | ENSCAFP00000008490 | ENSG00000182472 | ENST00000328867 | ENSP00000331636 |
| EED | ENSCAFG00000004471 | ENSCAFT00000007206 | ENSCAFP00000006672 | ENSG00000074266 | ENST00000263360 | ENSP00000263360 |
| | | | | | (11 other | |
| | | | | | variants) | |
| ENSCAFG00000002808 | ENSCAFG00000002808 | ENSCAFT00000004500, | ENSCAFP00000004160, | | | |
| | | ENSCAFT00000004496 | ENSCAFP00000004156 | | | |
| ENSCAFG00000005301 | ENSCAFG00000005301 | ENSCAFT00000008557 | ENSCAFP00000007929 | | | |
| ENSCAFG00000017000 | ENSCAFG00000017000 | ENSCAFT00000026931 | ENSCAFP00000025034 | | | |
| ENSCAFG00000024393 | ENSCAFG00000024393 | ENSCAFT00000022701 | ENSCAFP00000021084 | | | |
| ENSCAFG00000025839 | ENSCAFG00000025839 | ENSCAFT00000040122 (NPP) | | | | |
| ENSCAFG00000027866 | ENSCAFG00000027866 | ENSCAFT00000042149 (NPP) | | | | |
| L3MBTL2 | ENSCAFG00000001120 | ENSCAFT00000001714 | ENSCAFP00000001579 | ENSG00000100395 | ENST00000216237 | ENSP00000216237 |
| | | | | | (8 | |
| | | | | | other | |
| | | | | | variants) | |
| LOC483566 | ENSCAFG00000025561 | ENSCAFT00000039804, | ENSCAFP00000035704, | | | |
| | | ENSCAFT00000039802, | ENSCAFP00000035702, | | | |
| | | ENSCAFT00000039800, | ENSCAFP00000035699, | | | |
| | | ENSCAFT00000039792, | ENSCAFP00000035691, | | | |
| | | ENSCAFT00000039791 | ENSCAFP00000035690 | | | |
| MAPKBP1 | ENSCAFG00000009695 | ENSCAFT00000015402 | ENSCAFP00000014253 | ENSG00000137802 | ENST00000457542 | ENSP00000397570 |
| | | | | | (17 other | |
| | | | | | variants) | |
| NCAPH2 | ENSCAFG00000000603 | ENSCAFT00000000932, | ENSCAFP00000000851, | ENSG00000025770 | ENST00000420993 | ENSP00000410088 |
| | | ENSCAFT00000000931 | ENSCAFP00000000850 | | (15 other | |
| | | | | | variants) | |
| PPP6C | ENSCAFG00000020203 | ENSCAFT00000032176 | ENSCAFP00000029962 | ENSG00000119414 | ENST00000373547 | ENSP00000362648 |
| | | | | | (4 | |
| | | | | | other | |
| | | | | | variants) | |
| Q597P9_CANFA | ENSCAFG00000010423 | ENSCAFT00000016552 | ENSCAFP00000015315 | | | |
| SGIP1 | ENSCAFG00000018570 | ENSCAFT00000029488, | ENSCAFP00000027410, | ENSG00000118473 | ENST00000371037 | ENSP00000360076 |
| | | ENSCAFT00000029487, | ENSCAFP00000027409, | | (17 other | |
| | | ENSCAFT00000029485, | ENSCAFP00000027407, | | variants) | |
| | | ENSCAFT00000029483, | ENSCAFP00000027405, | | | |
| | | ENSCAFT00000029482 | ENSCAFP00000027404 | | | |
| XM_533169.2 | ENSCAFG00000007649 | ENSCAFT00000012229 (NPP) | | | | |
| XM_533289.2 | ENSCAFG00000003796 | ENSCAFT00000006097 | ENSCAFP00000005642 | ENSG00000101367 | ENST00000375571 | ENSP00000364721 |
| XM_541386.2 | ENSCAFG00000024642 | ENSCAFT00000037998, | ENSCAFP00000033655, | | | |
| | | ENSCAFT00000037993, | ENSCAFP00000033650, | | | |
| | | ENSCAFT00000037987 | ENSCAFP00000033643 | | | |
| XM_843895.1 | ENSCAFG00000012296 | ENSCAFT00000019526 (NPP) | | | | |
| XM_844292.1 | ENSCAFG00000023944 | ENSCAFT00000037096, | ENSCAFP00000032544, | | | |
| | | ENSCAFT00000037094, | ENSCAFP00000032542, | | | |
| | | ENSCAFT00000037093 | ENSCAFP00000032541 | | | |
| C1GALT1 | ENSCAFG00000002227 | | | NSG00000106392 | | |
| RPL6 | NSCAFG00000008873 | | | ENSG00000089009 | | |
| | ENSCAFG00000016065 | | | | | |
| PIK3R6 | ENSCAFG00000017382 | | | ENSG00000174083 | | |
| | ENSCAFG00000029323 | | | | | |
| XLOC_011971 | | | | | | |
| FGFR4 | ENSCAFG00000016518 | | | ENSG00000160867 | | |
| | | | | ENSG00000066468 | | |
| SCARA5 | ENSCAFG00000008354 | | | ENSG00000168079 | | |
| GFRA2 | ENSCAFG00000010049 | | | ENSG00000168546 | | |
| | ENSCAFG00000013622 | | | | | |
| CD5L | ENSCAFG00000016447 | | | ENSG00000073754 | | |
| XLOC_102336 | | | | | | |
| CXL10 | | | | | | |
| SLC25A48 | ENSCAFG00000001085 | | | ENSG00000145832 | | |
| KRT24 | ENSCAFG00000016017 | | | ENSG00000167916 | | |
| RP11-10N16.3 | | | | ENSG00000232298 | | |
| HIST1H | | | | | | |
| HS3ST3B1 | ENSCAFG00000028975 | | | ENSG00000125430 | | |
| C1GALT1 | ENSCAFG00000002227 | | | ENSG00000106392 | | |
| RPL6 | ENSCAFG00000008873 | | | ENSG00000089009 | | |
| PIK3R6 | ENSCAFG00000017382 | | | ENSG00000174083 | | |
| XLOC_011971 | | | | | | |
| FGFR4 | ENSCAFG00000016518 | | | ENSG00000160867 | | |
| | | | | ENSG00000066468 | | |
| SCARA5 | ENSCAFG00000008354 | | | ENSG00000168079 | | |
| GFRA2 | ENSCAFG00000010049 | | | ENSG00000168546 | | |
| KIAA1377 | ENSCAFG00000015131 | | | ENSG00000110318 | | |
| GRM5 | ENSCAFG00000004381 | | | ENSG00000168959 | | |
| GPC3 | ENSCAFG00000018864 | | | ENSG00000147257 | | |
| | ENSCAFG00000030890 | | | | | |
| FABP4 | ENSCAFG00000025410 | | | ENSG00000170323 | | |
| HTR4 | ENSCAFG00000018345 | | | ENSG00000164270 | | |
| U2 | | | | | | |
| CD300A | ENSCAFG00000032631 | | | ENSG00000167851 | | |
| Q95J95 | ENSCAFG00000013694 | | | | | |
| ZNF662 | ENSCAFG00000005374 | | | ENSG00000182983 | | |
| XLOC_026187 | | | | | | |
| MPO | ENSCAFG00000017474 | | | ENSG00000005381 | | |
| KIF5C | ENSCAFG00000005610 | | | ENSG00000262907 | | |
| | | | | ENSG00000168280 | | |
| CACNA1D | ENSCAFG00000008525 | | | ENSG00000157388 | | |
| XLOC_044225 | | | | | | |
| XLOC_067564 | | | | | | |
| NETO1 | ENSCAFG00000000031 | | | ENSG00000166342 | | |
| RGS13 | ENSCAFG00000030137 | | | ENSG00000127074 | | |
| COL6A6 | ENSCAFG00000006035 | | | ENSG00000206384 | | |
| KIAA1456 | ENSCAFG00000006759 | | | ENSG00000170941 | | |
| | | | | ENSG00000250305 | | |
| ADAMTS2 | ENSCAFG00000000334 | | | ENSG00000087116 | | |
| CD5L | ENSCAFG00000016447 | | | ENSG00000073754 | | |
| XLOC_102336 | | | | | | |
| CXL10 | | | | | | |
| SLC25A48 | ENSCAFG00000001085 | | | ENSG00000145832 | | |
| KRT24 | ENSCAFG00000016017 | | | ENSG00000167916 | | |
| HS3ST3B1 | ENSCAFG00000028975 | | | ENSG00000125430 | | |
| CCR6 | ENSCAFG00000030966 | | | ENSG00000112486 | | |
| XLOC_083025 | | | | | | |
| | ENSCAFG00000028509 | | | | | |
| PADI4 | ENSCAFG00000015768 | | | ENSG00000159339 | | |
| XLOC_022131 | | | | | | |
| XLOC_068212 | | | | | | |
| PROK2 | | | | ENSG00000163421 | | |
| XLOC_088759 | | | | | | |
| GZMA | ENSCAFG00000006735 | | | ENSG00000145649 | | |
| OBSL1 | ENSCAFG00000015641 | | | ENSG00000124006 | | |
| KIAA1598 | ENSCAFG00000011908 | | | ENSG00000187164 | | |
| U6 | | | | | | |
| NPDC1 | ENSCAFG00000019525 | | | ENSG00000107281 | | |
| PGBD5 | ENSCAFG00000012098 | | | ENSG00000177614 | | |
| XLOC_094643 | | | | | | |
| LBH | ENSCAFG00000005309 | | | ENSG00000213626 | | |
| GPR27 | | | | ENSG00000170837 | | |
| PTPN22 | ENSCAFG00000009255 | | | ENSG00000134242 | | |
| CSF1 | ENSCAFG00000019798 | | | ENSG00000184371 | | |
| KLRK1 | ENSCAFG00000025596 | | | ENSG00000255819 | | |
| | | | | ENSG00000213809 | | |
| CNNM1 | ENSCAFG00000009428 | | | ENSG00000119946 | | |
| B6F250 | ENSCAFG00000003692 | | | | | |
| | ENSCAFG00000029236 | | | | | |
| CD8A | ENSCAFG00000007464 | | | ENSG00000153563 | | |
| | ENSCAFG00000031437 | | | | | |
| GALNT13 | GALNT13 | | | ENSG00000144278 | | |
| EXTL1 | ENSCAFG00000012712 | | | ENSG00000158008 | | |
| RAB19 | ENSCAFG00000003959 | | | ENSG00000146955 | | |
| XLOC_100547 | | | | | | |
| CCL22 | ENSCAFG00000032287 | | | ENSG00000102962 | | |
| | ENSCAFG00000031494 | | | | | |
| MT1 | ENSCAFG00000009113 | | | | | |
| EOMES | ENSCAFG00000005510 | | | ENSG00000163508 | | |
| XLOC_091705 | | | | | | |
| RP11-664D7.4 | | | | ENSG00000248801 | | |
| TNFAIP3 | ENSCAFG00000000267 | | | ENSG00000118503 | | |
| FAM190A | ENSCAFG00000009949 | | | ENSG00000184305 | | |
| XLOC_077615 | | | | | | |
| | ENSCAFG00000029651 | | | | | |
| GZMK | ENSCAFG00000018379 | | | ENSG00000113088 | | |
| GZMB | ENSCAFG00000025287 | | | ENSG00000100453 | | |
| CCDC168 | ENSCAFG00000025243 | | | ENSG00000175820 | | |
| MARCKSL1 | ENSCAFG00000010588 | | | ENSG00000175130 | | |
| MAPK11 | | | | ENSG00000185386 | | |
| TRBC2 | ENSCAFG00000014478 | | | ENSG00000211772 | | |
| | | | | ENSG00000260881 | | |
| SCN2A | ENSCAFG00000011130 | | | ENSG00000136531 | | |
| CD151 | ENSCAFG00000023924 | | | ENSG00000177697 | | |
| TBXA2R | ENSCAFG00000019175 | | | ENSG00000006638 | | |
| TNFRSF21 | ENSCAFG00000002078 | | | ENSG00000146072 | | |
| | ENSCAFG00000029467 | | | | | |
| NKG7 | ENSCAFG00000002845 | | | ENSG00000105374 | | |
| CHGA | ENSCAFG00000024864 | | | ENSG00000100604 | | |
| CCL5 | ENSCAFG00000018171 | | | ENSG00000161570 | | |
| H6BA90 | ENSCAFG00000016175 | | | | | |
| PLEKHG5 | ENSCAFG00000019602 | | | ENSG00000171680 | | |
| SMOC1 | ENSCAFG00000016602 | | | ENSG00000198732 | | |
| TNIK | ENSCAFG00000015157 | | | ENSG00000154310 | | |
| CCL19 | ENSCAFG00000001954 | | | ENSG00000172724 | | |
| | ENSCAFG00000028940 | | | | | |
| XLOC_024761 | | | | | | |
| RGS10 | ENSCAFG00000031204 | | | ENSG00000148908 | | |
| TMPRSS13 | ENSCAFG00000012845 | | | ENSG00000137747 | | |
| DLGAP3 | ENSCAFG00000003602 | | | ENSG00000116544 | | |
| | ENSCAFG00000028850 | | | | | |
| | ENSCAFG00000030894 | | | | | |
| SLC38A11 | ENSCAFG00000010662 | | | ENSG00000169507 | | |
| KEL | ENSCAFG00000003658 | | | ENSG00000197993 | | |
| | | | | ENSG00000260040 | | |
| ABCA4 | ENSCAFG00000020121 | | | ENSG00000198691 | | |
| TNFRSF18 | ENSCAFG00000019329 | | | ENSG00000186891 | | |
| TNFRSF4 | ENSCAFG00000019328 | | | ENSG00000186827 | | |
| AFF2 | ENSCAFG00000019094 | | | ENSG00000155966 | | |
| | | | | ENSG00000269754 | | |
| CXCR3 | ENSCAFG00000017146 | | | ENSG00000186810 | | |
| TCTEX1D4 | ENSCAFG00000004701 | | | ENSG00000188396 | | |
| FBXO11 | ENSCAFG00000002669 | | | ENSG00000138081 | | |
| CHRM4 | ENSCAFG00000009203 | | | ENSG00000180720 | | |
| CD8B | ENSCAFG00000007461 | | | ENSG00000172116 | | |
| HTRA1 | ENSCAFG00000012556 | | | ENSG00000166033 | | |
| LAT | ENSCAFG00000017333 | | | ENSG00000213658 | | |
| LAD1 | ENSCAFG00000030784 | | | ENSG00000159166 | | |

### EXAMPLES

### Example 1

### Identification of Germ-Line Risk Factors for B-Cell LSA and HSA

To search for inherited (i.e. germ-line) risk factors predisposing to B-cell LSA or HSA, a Genome-Wide Association Study (GWAS) was performed on golden retrievers. DNA was extracted from whole blood samples taken from 43 B-cell LSA cases, 148 HSA cases, and 190 healthy controls>10 years of age were genotyped using the Illumina 170K canine HD array (CamFam2.0, Illumina, San Diego, Calif.).

Since the dog population contained high levels of encrypted relatedness and complex family structures, it was necessary to apply a method that could successfully control for the population stratification present in this data set (see FIG. 1, Price et al 2010). In brief, the dataset was analyzed in three steps. First, PLINK (Purcell et al, 2007) was used to apply standard quality filters including genotyping rate per single nucleotide polymorphism (SNP, >95%) and per individual (>95%), and minor allele frequency (MAF, >1%). Secondly, GCTA (Yang et al, 2011) was used to estimate a genetic relationships matrix to remove excessively related individuals, and also to calculate the principal components of the whole-genome SNP genotype data per individual by the EIGENSTRAT method (Price et al 2006), which was used as a covariate in the final step. Finally, EMMAX (Kang et al, 2010) was used to test for the disease-genotype association with adjustment for the identity-by-state (IBS) matrix calculated by EMMAX and for the first principal component calculated by GCTA. This resulted in the final dataset of 127,188 SNPs from 145 HSA cases, 42 B-cell LSA cases, and 186 controls for the association analysis.

Chromosome 5 was identified with significant association to disease status (FIG. 2, p=3.5×10⁻⁷). More specifically, 19 disease-associated SNPs were found to be located on chromosome 5 between 32 Mb and 37 Mb. The top two SNPs identified, BICF2G63035726 (at position 32,901,346 [32.9 Mb] based on CamFam2.0) and BICF2G630183630 (at position 36,848,237 [36.8 Mb] based on CamFam2.0), had P values of 3.5×10⁻⁷ and 4.2×10⁻⁷, respectively (Table 4). BICF2G63035726 was found to be part of the linkage disequilibrium (LD) region ch5:32.5 Mb˜33.1 Mb, and BICF2G630183630 was found to be part of the LD region ch5:36.6 Mb˜37.3 Mb (FIG. 3).

Among the dogs with LSA, 88% of the dogs had at least one risk allele (19 dogs/45%+/+, 18 dogs/43%+/−, and 5 dogs/12%−/−. Among the dogs with HSA, 94% of the dogs have at least one risk allele (78 dogs/53%+/+, 60 dogs/41%−/−, and 10 dogs/7%−/−). Frequency of the risk alleles is shown in FIGS. 3C and 3D.

TABLE 4
Location and LD regions associated with the top two disease-associated SNPs

| | | Position | Alleles | | | | |
|---|---|---|---|---|---|---|---|
| | | (bp, | (non-risk/ | MAF | MAF | Odds | P |
| LD Region | SNP ID | CamFam2.0) | risk) | cases | control | Ratio | value |
| chr5: 32.5Mb~33.1Mb | BICF2G63035726 | 32,901,346 | T/C | 0.29 | 0.49 | 2.36 | 3.52E−07 |
| chr5: 36.6Mb~37.3Mb | BICF2G630183630 | 36,848,237 | C/T | 0.23 | 0.09 | 3.07 | 4.20E−07 |

### Identification of Candidate Genes in the Associated Regions

Several genes were found to be located within the two disease-associated regions ch5:32.5 Mb˜33.1 Mb and ch5:36.6 Mb˜37.3 Mb. These genes include C11orf7, ANGPTL5, TRPC6, KIAA1377, NTN1, NTN3, STX8, WDR16, USP43, GLP2R, novel transcript chr:5:32732962-32766974 (SEQ ID NO:1) and DHRS7C (FIG. 3). The two disease associated regions were further analyzed for potential candidate genes located within or near these regions that could predispose dogs to HSA or LSA.

First, whole genome sequencing was performed on tumor and normal DNA paired samples form 3 B-cell LSA dogs, and the coding exons of genes within the associated regions were examined to find germ-line mutations that associated with the risk haplotypes at the 32.9 and 36.8 Mb loci. Three genes, KIAA1377, ANGPTL5, and TRPC6, were found to have germ-line mutations within the coding sequence associated with the risk haplotypes and are summarized in Table 5.

TABLE 5
Genes found within the disease-associated regions
with germ-line mutations in the coding sequence

| | | # of | # of | | |
|---|---|---|---|---|---|
| | | Germ-line | Somatic | | |
| Gene | # of | candidate | muta- | Frequency | Type of |
| name | Mutations | mutations | tions | in sample | mutation |
| KIAA1377 | 1 | 1 | 0 | ⅔ | non- |
| | | | | | synonymous |
| ANGPTL5 | 1 | 1 | 0 | ⅔ | non- |
| | | | | | synonymous |
| TRPC6 | 1 | 1 | 0 | ⅓ | non- |
| | | | | | synonymous |

Secondly, gene expression profiles of tumor samples from nine B-cell LSA dogs were generated using the Affymetrix canine expression array (Affymetrix, Santa Clara, Calif.). The nine dogs were divided into two groups (risk vs. non-risk) defined by the genotypes of the 32.9 Mb or the 36.8 Mb top SNPs. The risk group defined by the 32.9 Mb SNP contained 5 homozygotes for the risk allele (T/T), and non-risk group included 3 heterozygotes (T/C) and 1 homozygote for the non-risk allele (C/C). The risk group for the 36.8 Mb SNP included 4 heterozygotes (T/C) and 1 homozygote for the risk allele (T/T), and non-risk group included 4 homozygotes (C/C) for the non-risk allele. The expression data was analyzed by the methods described below to detect differentially expressed genes between the risk and non-risk groups.

Following hybridization on the array described above, each chip passed quality assurance and control procedures using the Affymetrix quality control algorithms provided in Expressionist Refiner module (Genedata AG, Basel, Switzerland). Probe signal levels were quantile-normalized and summarized using the GeneChip-Robust Multichip Averaging (CG-RMA) algorithm. Normalized files were imported into the Expressionist Analyst module for principal component analysis (PCA), unsupervised clustering, and to assess significant differences in gene expression. There are no precise tests to develop sample size estimates for gene expression profiling, theoretical principles and empirical observations were applied to support the sample size for these experiments a priori. The Power Atlas available online, provided an empirical estimate that the sample sets used for these experiments should have provided >90% power at p=0.05 to identify true positives, although the power to identify true negatives could have been be lower. The correlation coefficient (r2) for expression values of all probes between duplicated samples was >0.95. Probe IDs were mapped to corresponding canine Entrez Gene IDs using Affymetrix NetAffx EntrezGene Annotation. Prior to hierarchical clustering, normalized chip data were median-centered and log 2-transformed. Supervised groups included all of the tumors available for each defined genotype. Two group t-tests were done to determine genes that were differentially expressed between groups.

Data were confirmed by quantitative real time reverse transcriptase-polymerase chain reaction (qRT-PCR) using 11 samples where 4 had been included in the original set of 9 dogs on the array and 7 were independent of the samples from the array dataset. The genes that had a P value less than 0.05 and greater than two-fold difference between the two groups were considered significant. The genes located within 1 Mb from, or in between the two SNPs were considered differentially expressed if the P value was less than 0.05 regardless of fold-change.

This analysis identified eight candidate genes, ZBTB4, BIRC3, CD68, CHD3, CHRNB1, MYBBP1A, RANGRF, and ANGPTL5, located at or proximal to the disease associated regions, as differentially expressed (FIG. 5) and 141 genes (genome-wide) as differentially expressed between the risk and non-risk groups.

These 141 genes are ABTB1, AGA, AK1, ANXA1, B4GALT3, BAG3, BAT1, BCAT2, BEX4, BID, BIRC3, BTBD9, CCDC134, CCDC18, CCDC88C, CD1C, CD320, CD68, CDKN1A, CMTM8, COASY, COL7A1, CPT1B, CTSD, DDX41, DENND4B, DGKA, DHRS1, DUSP6, ECM1, EFCAB3, EIF4B, LOC478066, FABP3, FADS1, FBXL6, FBXO11, FBXO33, FBXW7, FNBP4, GALNT6, GBE1, GDPD3, GNGT2, GPR137B, GSTM1, GTF2IRD2, GTF3C3, GUCY1B3, HBD, LOC609402, ICAM4, IRF5, KIF5C, KLHDC1, KLHDC9, LBX2, LOC475952, LOC479273, LOC479683, LOC482085, LOC482088, LOC482361, LOC482532, LOC482790, LOC483843, LOC484249, LOC484784, LOC485196, LOC487557, LOC487994, LOC490377, LOC490693, LOC491116, LOC609521, LOC610353, LOC610841, LOC611771, LOC612387, LOC612917, LOXL3, LZTS2, MED24, MFSD6, MTA3, MYC, MYO19, NAGA, NAPRT1, NEIL1, NQO2, OGT, OSGEPL1, OVGP1, P2RX5, PDLIM7, PER3, PHKA2, PIGV, PIK3R6, PITPNM1, PRKRA, PVRIG, RAB24, RAB25, RABEP2, RASAL1, RBM11, RBM18, RBM35A, RBPJ, REC8, RILPL1, RPA1, SIGLEC12, SLC37A1, SP1, SUOX, TIMM22, TIPARP, TLE4, TMED6, TMEM41B, TNFSF8, TRAF5, TRMT1, TTC39C, TTF1, TUBB2A, UNC93B1, YWHAE, ZFAND2A, ZFC3H1, ZMAT1, ZMYM1, ZNF215, ZNF292, ZNF331, ZNF513, ZNF608, ZNF674, and ZNF711.

### Example 2

### Identification of Somatic Mutations Associated with LSA

### Methods

Whole-genome sequencing of lymphomatic tumors as well as a matched normal blood sample from six dogs diagnosed with canine lymphoma was performed with the goal of identifying candidate somatic mutations, including SNPs, insertion/deletion events (indels), copy number variants (CNVs) and structural rearrangements. Four tumors were of B-cell type LSA and two were of T-cell type LSA.

For each sample, approximately 1 billion 101 base-pair paired-end reads were generated using Illumina HiSeq (Illumina, San Diego, Calif.), of which 98% were aligned to the CanFam2.0 reference genome with the Picard pipeline (Broad Institute, Cambridge, Mass.). The resulting depth of coverage per sample was roughly 40×. Genotype calls were made from the aligned reads using the Genome Analysis Toolkit's (GATK, Broad Institute, Cambridge, Mass.) UnifiedGenotyper in multi-sample mode. Hard filters were applied for low quality, strand bias, clustering, and excessive read depth. Accurate genotype calls were possible at 95% of the bases in the genome. Alignments were subsequently cleaned around novel insertion/deletion events in accordance with the GATK Best Practices guide.

Comparison of the SNP calls produced by the UnifiedGenotyper to GWAS data from the same samples showed greater than 99% concordance at common sites in 11 of the 12 samples. One tumor sample did not match its GWAS data, although the matched normal from the same sample did. Both the tumor sample and the matched normal sample were excluded from further analysis.

Somatic SNP variants were called with the MuTect software package v1.0.18339 (Broad Institute, Cambridge, Mass.) using standard practices and the pathology-based estimates of tumor purity provided in the table below. Somatic insertion/deletion variants were called using the GATK's SomaticIndelDetctor in ‘somatic’ mode. Results were filtered using standard practices. Both somatic SNP and indel variants were then annotated with the software package snpEff (“SNP effect predictor”; which is available at the snpeff website through sourceforge) using the CanFam2.61 (ENSEMBL version 61) gene annotation database.

Passing variants were segregated into two bins. Those variants observed in only a tumor sample were declared somatic candidate mutations. Those variants observed in both a tumor sample and its matched normal were declared germ-line variation. A second filter was then applied to the list of both germ-line variation and somatic candidate mutations such that the confidence of the reported genotype call exceeded that of finished sequence. The freely available software snpEff (“SNP effect predictor”; snpeff.sourceforge.net) was used to annotate the variants with snpEff's CanFam2.61 (ENSEMBL version 61) annotation database.

### Results

Several genes were identified with somatic mutations associated with LSA. These genes include TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1. Several of these genes, TRAF3, FBXW7, DOK6, RARS, JPH3, LRRN3, MLL2, OGT, POU3F4, SETD2, CACNA1G, DSCAML1, MLL, were known previously to occur in human lymphoma or leukemia. These genes were found to have disease-associated somatic mutations in dogs and are summarized in Table 6.

TABLE 6
Genes associated with human lymphoma or leukemia that contain somatic
mutations in LSA samples collected from golden retrievers

| | Genomic | | | |
|---|---|---|---|---|
| Common | Location | Frequency | Also occurs | |
| name | (CanFam 2.0) | in samples | in human | Result of Mutation |
| FBXW7 | 15:53199637- | ⅗ | Lymphoma | Amino acid substitutions (2), |
| | 53297430 | | Leukemia | Premature stop |
| CACNA1G | 9:29805671- | ⅕ | Lymphoma | Amino acid substitution |
| | 29868112 | | Leukemia | |
| DSCAML1 | 5:19091789- | ⅕ | Lymphoma | Amino acid substitution |
| | 19192555 | | Leukemia | |
| MLL | 5:18223809- | ⅕ | Lymphoma | Frame shift |
| | 18310951 | | Leukemia | |
| TRAF3 | 8:73804434- | ⅖ | Lymphoma | Frame shift, Amino acid |
| | 73832278 | | | substitution, Premature stop |
| JPH3 | 5:68451434- | ⅕ | Lymphoma | Amino acid substitution |
| | 68530493 | | | |
| LRRN3 | 14:53886322- | ⅕ | Lymphoma | Amino acid substitution |
| | 53888693 | | | |
| MLL2 | 27:8531732- | ⅕ | Lymphoma | Frame shift |
| | 8572773 | | | |
| OGT | X:58747315- | ⅕ | Lymphoma | Frame shift |
| | 58782084 | | | |
| POU3F4 | X:67483018- | ⅕ | Lymphoma | Amino acid substitution |
| | 67484103 | | | |
| SETD2 | 20:44710232- | ⅕ | Lymphoma | Frame shift |
| | 44799043 | | | |
| DOK6 | 1:11456167- | ⅕ | Leukemia | Amino acid substitution |
| | 11709626 | | | |
| RARS | 4:46435613- | ⅕ | Leukemia | Amino acid substitution |
| | 46458784 | | | |

The remaining genes, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, have not been identified in association with human lymphoma or leukemia but were found to have somatic mutations associated with canine LSA (Table 7).

TABLE 7
Genes that contain somatic mutations in LSA
samples collected from golden retrievers

| | Genomic | | |
|---|---|---|---|
| | Location | Frequency | |
| Common name | (CanFam 2.0) | in samples | Result of Mutation |
| ADD2 | 10:72213503- | ⅕ | Amino acid substitution |
| | 72245100 | | |
| ARID1A | 2:76224444- | ⅕ | Amino acid substitution |
| | 76292627 | | |
| ARNT2 | 3:59871792- | ⅕ | Amino acid substitution |
| | 59983630 | | |
| CAPN12 | 1:117252707- | ⅕ | Frame shift |
| | 117264668 | | |
| EED | 21:16258069- | ⅕ | Amino acid substitution |
| | 16287537 | | |
| ENSCAFG00000002808 | 13:61588099- | ⅕ | Amino acid substitution |
| | 61661603 | | |
| ENSCAFG00000005301 | 16:24614753- | ⅕ | Frame shift |
| | 24686356 | | |
| ENSCAFG00000017000 | 4:45571611- | ⅕ | Amino acid substitution |
| | 45574208 | | |
| ENSCAFG00000024393 | 16:62455232- | ⅕ | Amino acid substitution |
| | 62457524 | | |
| ENSCAFG00000025839 | 11:35540786- | ⅕ | Amino acid substitution |
| | 35541109 | | |
| ENSCAFG00000027866 | 17:43160406- | ⅕ | Amino acid substitution |
| | 43160525 | | |
| L3MBTL2 | 10:27065825- | ⅕ | Amino acid substitution |
| | 27085050 | | |
| LOC483566 | 18:43341294- | ⅕ | Amino acid substitution |
| | 43343499 | | |
| MAPKBP1 | 30:11801201- | ⅕ | Amino acid substitution |
| | 11849823 | | |
| NCAPH2 | 10:19778049- | ⅕ | Amino acid substitution |
| | 19789727 | | |
| PPP6C | 9:61225428- | ⅕ | Amino acid substitution |
| | 61260767 | | |
| Q597P9_CANFA | 25:42853852- | ⅕ | Amino acid substitution |
| | 42958217 | | |
| SGIP1 | 5:46748758- | ⅕ | Amino acid substitution |
| | 46862013 | | |
| XM_533169.2 | 18:40592009- | ⅕ | Amino acid substitution |
| | 40593998 | | |
| XM_533289.2 | 19:12678157- | ⅕ | Amino acid substitution |
| | 12679076 | | |
| XM_541386.2 | 1:104332009- | ⅕ | Amino acid substitution |
| | 104334024 | | |
| XM_843895.1 | 7:13297370- | ⅕ | Premature stop |
| | 13297789 | | |
| XM_844292.1 | 8:77227023- | ⅕ | Amino acid substitution |
| | 77227897 | | |

TABLE 8
Nucleotide Sequence of SEQ ID NO: 1 (chr5: 32732962-32766974). The
underlined sequence represents a novel transcript (SEQ ID NO: 2)

TGTGCATGGTTGGTCCAGATTTGGGGTGTACGTGACTACCACTGTTTGTGTTATACATGATCAGGAGAATGAACAG
GAAGAATATGGAATCCACTTAACACATGTGGTGTCGCCAGGAGGAGCATGATTCTGCCAGCATTGACTAGACTTAT
CTCTGATATGTTTTCAATCTGCAAAACTGAGAAGCTAGTAAATTTGTATCAATTTAGACTCTTTTCTTCAATGAGA
AGTGAGTTGAAGGTGGGTGGATGGAGACCCTCTGTTTTCTTCTATCCATAAATTGCCAATATAGAAATTTATGCAT
ATGTATTTCATATTCATTTTTTTGATACAAGAAGAATGTCATGAACTTTATGTGGGTCATAGTTTTCTTCTTTTTT
TCCCCCTTAGCTTGCAACTTACTAAAAGAGTAAGTTGTTTTGCAAAGATCATTAGGTAGATTTTTTGTTTGTTTGT
TTGTTTTAATCGTGTATTTCTAATGTTTTGTGTGCAGAAAATCTTACTTGCTCTTTCCCAGCTGGAAATGATATCT
CTTATATATTTTTATTTTTGGATCTCTTTTATGACATGTTTCACTTCCTGTTTTAGTTCATAGATACCTAAATGTG
TTTTATTTTTCCTATTAGAATGTAAAACAGCCTTGAGAAATGATGTTTTATATACTGTATTTCCAGTATTTATAAA
TAGAATATATAAGTAAATTAAAAACTTCCCACTTTGAATAGCAACAGCAAAAAACATCTATAGTCCCTAAAAGAGA
ACAGAGATCCAGTGGAAAATGTTCCTGGAAAATGCAGCTCTGCTGTTTTCTGGGTACTAAGGAGCCTATGAGTTGG
TGTGACTTCAGGCAACATGTTCCTTCTAAGTTTTTTCACCTGTAAAATGAAAATATGTCTGCCTCATAGAACTGAT
GAGAAGGTCTAAGTTAGGAAACACATAGAACTCCAAGGTGCTATAGAACTGTTAAGAAACCACACCATATGCAAAC
GGGAATGTGGAGCTTTCACTCTCACATTTAACTTAGTTTACAGTCCTCAGGAGAAATATATAAGCCAAACATTAAG
TATATGGAAGAACATCAACCTCATAGTAGGAACTCAGAACCGAATTTGCTGAACTGAATTACTGTTAACTTCAATT
ATCCAAAAAGCTTCCCTTAGAGAGTCAAGGATGGAAGGCTCTAAGCTGGCACACAGGAATAGCGTTATTGGATTTG
TGCTCAGATCATGATTCCACAGAGCTTTGGATCCATCCCATAAGAGAGCTGTTGTTGGAAAGCACTGTGTAGGATT
TTACTTAGAGGAATCAGCATTGACTCCTTTTTTAATCGCTCAACAGAAGTGGCACTCAGAAATTACCTTTGAGTTA
TCCAGCTGCTGGAAATCTAAGAGCTGTCCAGAGGCAAGAAATACTTAAAACTTAGAGTGGTTAAGGTAAGTAGATG
CTGAAGACAATTTTCACTAGTGATTGCTGTCAACTGGAGTGATAGCAGTTGGTAAGACCTATTCCTTTATTGAACT
TATTCAATGCCTACTTGTGACAGCTGCTAGTTAGCCACAGGGATTCAGGTGATGGTAAGGATGAAAATCCCTTCCA
ACCCTTCAGAGTAGCTCTTGGAAGGTACTGTCACAAGCGCTGGCATCAATGAGTCCTGCACTAGTGGTGGCCTCCA
CACCCCAGGATCCATGAATGACTCACTCTGACAGAAGCACCTGTGGAGTAAAACCAGCGGTTTCCCCAACATCCAA
TATGAAGCATCCTAACATTCAGGAGAAGAATGATGATCACTGAGCTTTAAAAATAAACTTTTATTTTCCAAAAGGA
AAGTCATGGCTTGATTCCCTTGTCAATTGACACTGAGTAATTTCCTTCATCTGTGTGCAGTTCTCTCTTACATTGT
GTAGCACTTTCTAGCTTACACTGCAGCTAACTCTTCTTAAAGGGAAGAGAGGCTAGAATTAAGCATTGCTGGAAAA
ACTATATATGAAGATACTCATGCTTTAATAAGAAGTCTAAAATAGTCTCTAAAAAGTTGGGTGACTCATTAGTAAC
ATTCTCTTCCCACGGCCTCCTGCCATATAATTTTGACCTGCAGTGACCTCCCCTGTACTTTTAGATGGACATCAGC
ATCTAGTATTTATTATGGTACCTTGGAAAGATGCCAAAAGATCAAACAGAGCATGAAAATATTTGTAGGATGGAAA
TGCATTCTATAGACTCTTAAGTATCACTCTAAGAAGTTGATGTATTGGGTGAAATAAGAAAATTGAGATTACTTGC
TTACAAAACATAGGAGTTTTCTTTTAAAATTTTGTAATTCAGAATGGCTTGCTTACATTTTATGGATTTACTGGTT
ATAGCTTCAGATGAAGAGCATGGACCTTGAGGTCTGCCTGGTTGTGTTTTTTTTTTTTTTTTAGATTTTATTTATT
CATTCATGAGAGACAGAGAGAGAGAGAGAGAGAGAGGCAGAGACACAAGCAGAGGGAGAAGAAGGCTCCCTGCAGG
GAGCCCAATGTGGGACTCGATCCCAGGACTCCAAGATTACACCCTGAGCCAAAGGCAGATGCTCAACCACTGAGCC
ACCCAGGCATCCAAGCCTGCTTGTGTTTCAACATCATTTTTGGTACTTACCTTATAGATTGGTCCATTAGGAGAAT
CGAATAAAATAATGCATGTAAAGCATTTAGTAGAGTGCCATGCCTACATCCTAATAATCATATATAAATGTGCATT
CATGGTGATTTTGTTATTCCCAGTCATTTACATCTAAGTAATACGTTTGTTCATTCCCTACCATATACCTCAGGGA
GTGTATCATTCAATGTCATACTACTTTGTTCAAGAAACAATATTCTGTCAGTGATTGGCTGTTAAAGGGAGATTTA
AAGGCTTTTGGCCTAATTTTAGGACAGTTCTGAAAAGGCCTTTCAGCCTCAGAACAATCTGTTGGATTAGTTCAAG
TCTCATTTGTGACTTCATGTTAATGGGCCACCATAAATCTAACCAAGTAGGATGCCAAAAACAGTTGCTATGCTGG
ATATGGTGTCTTTGCTAGAGCAGGTTAACACTCTTTTGCCTGCATGGGATGTGAGTCATCGATATAGTAAATGCAT
TCTTGCGCTTACTCATCAAGAAAGAAGATTGGAAGCTAATCTCTTATAGTCACAAGAAAGGGTTAATAGTCTACAA
AATCTTACCCAAGGCTACATTAATCCTACTATTTGTCATAATTTAGTTCAAAGGGACCTGGGTTATCTTAAAATAC
TGCAGAAGACCATTGTTCCCCTATATTAATGATGTCATGTCAATGGGATGTGATGAGTAAGAGCTGGCACATATTC
TGAATGCTTTGGTAAAATATTTCCCGCTCCAAAAGGCAGAAAAAACCTGACATAGTTTTCTAGGAGAATTTAGTGA
CATGTAAAGTGTTTATGAATCTAATGGTCTGGGGTGTACCTTTACATACTTTCCAGAAAGTGGGGTGGTTATTGCT
CCTTGCCCCTAAAGAAGAAGCACAAAGCTTGGTAGGGCTTTTTTGGGGTTTGTAGTCAACACAGTCTCCACATGGG
GTTACTTTAATGCTCAATTTATTTATTGATATGAAAGCCTTTTCACCCTGAGAGATTCAGATCAAGATCAGGAAAG
GTCACTGTAGCTGGTCTAGTTTGCAAAACGAGCGGTTCTGCCACTTGGGCTATACAACCCAGCAGATCCCATAATT
CTGATGTTACCTGTGGTAAAGAGAGATGCTTGGACCCTGAACTTTATGGGCAACTAATATTCGATAAAGGAGGAAA
GACTATCCATTGGAAGAAAGACAGTCTCTTCAATAAATGGTGCTGGGAAAATTGGACATCCACATGCAGAAGAATG
AAACTAGACCACTCTCTTTCACCATACACAAAGATAAACTCAAAATGGATGAAAGATCTAAATGTGAGACAAGATT
CCATCAAAATCCTAGAGAAGAACACAGGCAACACCCTTTTTGAACTCGGCCATAGTAACTTCTTGCAAGATACATC
CACAAAGGCAAAAGAAACAAAAGCAAAAATGAACTATTGGGACTTCATCAAGATAAGAAGCTTTTGCACAGCAAAG
GATACAGTCAACAAAACTCAAAGACAACCTACAGAATGGGAGAAGATATTTGCAAATGACATATCAGATAAAGGGC
TAGTTTCCAAGATCTATAAAGAACTTATTAAACTCAACACCAAAGAAACAAACAATCCAATCATGAAATGGGCAAA
AGACATGAACAGAAATCTCACAGAGGAAGACATAGACATGGCCAACATGCATATGAGAAAATGCTCTGCATCACTT
GCCATCAGGGAAATACAAATCAAAACTACAATGAGATACCACCTCACACCAGTGAGAATGGGGAAAATTAACAAGG
CAGGAAACAACAAATGTTGGAGAGGATGCGGAGAAAAGGGAACCCTCTTACACTGTTGGTGGGAATGTGAACTGGT
GCAGCCACTCTGGAAAACTGTGTGGAGGTTCCTCAAAGAGTTAAAAATAGACCTGCCCTACGACCCAGCAATTGCA
CTGTTGGGGATTTACCCCAAAGATACAAATGCAATGAAACGCCGGGACACCTGCACCCCGATGTTTCTAGCAGCAA
TGGCCACTATAGCCAAACTGTGGAAGGAGCCTCGGTGTCCAACGAAAGATGAATGGATAAAGAAGATGTGGTTTAT
GTATACAATGGAATATTACTCAGCTATTAGAAATGACAAATACCCACCATTTGCTTCAACGTGGATGGAACTGGAG
GGTATTATGCTGAGTGAAGTAAGTCAGTTGGAGAAGGACAAACATTATATGTTCTCATTCATTTGGGGAATATAAA
TAATAGTGAAAGGGAAAATAAGGGAAGGGAGAAGAAATGTGTGGGAAATATCAGAAAGGGAGACAGAACGTAAAGA
CTGCTAACTCTGGGAAACGAACTAGGGGTGGTAGAAGGGGAGGAGGGCGGGGGGTGGGAGTGAATGGGTGACGGGC
ACTGGGTGTTATTCTGTATGTTAGTAAATTGAACACCAATAAAAAAAAAATAAATAAAGAGAGATGCTGTTTGGAG
TTTCTGGAAGACCAAAGAGAGAAACACAGTACAGCCCCATAGAGTTCCAGAGTAAGGACAAGCCTATATAACTATA
GACGATTCCTTTTATTTTTAAGATGAAACTGAGATTTATTTATTTTTTATAAATTTATTTTTTATTGGTGTTCAAT
TAGCCAACATATAGAATAACACCCAGTGCTCATCCCGTCAAGTGCCCACCTCAGTGCCCGTCATCACCCAGTCACC
CCCACCCCCCACCCACTCCTTTTCCCCACCCCTAGTTCGTTTCCCAGTTAGGAGTCTTTCATGCTCTGTCTCCCTT
TCTGATATTTCCCACTAATTTTTTCTCCTTTCCCCTTTATTCTCTTTCACTATTTTTTATATTCCCCAAACTATAG
ACGATTCCAACAGCTCTGGATGCTACTGGGCTCTAAGAGAAACCCAGTGTCTCACCATGGGGCCTATGTGACCACA
ATTCCAGAACTGCCCATCAGAGTTTGGCTGCTGCTAGATATACCAAGTCATAAGATCAGGTGAATGAGAAGCTACC
CTATGATGGGGGCACATCTCAGGGTTGGTGTAGAAGTCACCAGTAGGCTAACATGATAAGTGGCCCAGACACTCAT
CACCTACCACTATTGTGGATGCTTATTTTCAGCCTCAATTTATATATGACAGGTGATGGAGGGTGCCCCGTGACCA
GCTGATGAAGGAAGGAAACGGTTAACTTGATCACAAGGACGGTGGCTAAAAGTGGACACCCAACTCTCTACACCCC
CTTGAGGATGGCCTTGAAAACCAGGCAAGGTGAAATCCTCCTTGTGGGCAGAGCTGTGTGTGGAGGGGAAATTGTG
CAAGACAAGGGTGTGTGTGTGTCCATAGGGCATGGTGAAAAGTCTCCCTGGCTGGCTGGGGCTGTACAGGAAGAAA
ACTGGAGAAAGTCCAGGTCAGGAAGGGGCATTAATTTACCAGGCACATGGAATAAGCAGGGGTTAATTGCAAGCCT
CTGTCCTTGGCCACTGCCACACCTGTGCCATGACTGCAGCCATGGGAATGGTACAGTCATAGGGACGGCCCAGTCT
GCCCACCCCACCCCCCAGGCCGACCAACCAAAGACTGACCATTGTAAGACAGAAGAGGGCCCGGCCTCCTTCCATG
AGTGGTGCGTCTGCATCCAGAGCCTCTCACGGGATCGAGGATGGCCTTCAGATGGGACCTGGCCAGACATTCTGGC
AAGCTTATTTCTCCACCTTATCTTTCTTCCCTTACCTCCCTACTCTTGAGAACACTCCTTCAATAAATCACTTTAA
CAAGGATCCTGTTCTTGGGCTGTTTTCTGGATATCTGACCCAGGCCACCAGTCATCAGCATACAGTTGGTGTTTGG
AGCTGTGTGCATGAGTGAGATCCCTTAAGAGTGCAGAGTGACAGCAACAGTGGCTACAGGACAGTGAGCGGCAGGA
CTGCTGAGTCACTGGGAATGTGGGACATAAACCTACATAGAAGCCAAGACAATGTTTCAGGAATGAAAAGGCAGTT
AGAGTGTCAATGGGAGAAAGTTCCACAGGTGCTGTGGTGACCTCAGCAGATTCCCCTGGTTCTGGGTGCTGTGGAA
GGTCCAGCAATCACCCCAGTGTCTGACCTCGGGGCAAGGGCTCTGATCCTTTGCTGCAGGTGGAACAATGCTGTAG
TTCGTGCTCCAGAGTTCCCTGGGGGAACAGGTGGAGGCTCAGTATCACCTGAAGCTGCATCCTTGTCTCTTCTTCA
TCTGGAAACCTCACTCCCTTATAGACTCCTCCCGAGAGCATGGGCTCCTAGAAACTGAGCCTGGGGTTATGATTCT
GGGGATTCGTTAAAGGATCGCTAAGACAAGAATTCAAGGAAAACAAGTATCAAAATGTGTTTTTAGCATATGGAAA
CTAGGATGTAACTAGTAAAATGGGGCAAAAGTCAGAGTAGACAGAGAAATGAATAGAAGGTGAGTCAAAGAAGTCC
ATGGGTTCAGACAAGTGCACCAAGCACTTTGTTGTGAAACCAAGCAAAGCCTTTATGGTAGATAGATTTGGAAACA
GGGTAAAAACAGATGCGTGTTCATGTGTACACATGCGTGTACATGTTGTAAGACATGGCATTGAGTGCTCTAAGTT
GAGAGAAAATAACCAACAAGAAGGAAGAGGTTGATAATCCAGGAAAAGAGGTGGAACCAGTATCACTGAACACCTG
TGAGATCTTGTAAAAGTTTTTGTGATGAAAAACCACAAACTTGGGGGATGCCTGGGTGGCTCAGTGGTTGAGTATC
TGCCTTCGGCTCAGGTTGTGATCTCAGGGTCCTGGGACAGAGTCCCACATTGTGCTCCCTATGAGGAGCCTGCTTC
TCCCTCTGCCTATGTCTCTGCCTCTCTCTGTGTCTCTCATAAATAAAGAAAATCTTAAAAAATAGTAAAAAAGTAA
AAAGTAAAAACCACAAACTCAGAATAAAGGATGAGTGGCAACATATTTTATTTTATATTTAATAAAAGTTTTTGCT
GTTATAGTCTCACATACTCAGATGTATCCAAACCTCCTGAATAGATAATAATGATAATTTTCTGCAAAATGGATGA
GCAGGACTTTAAAAATAATGGGTTTCTTATTGCCTGACTTGTGCCATGTAAAATATATTTCCTGTTGCTAGTGTCA
GATGTGATAAACTATAAAGCAAGTGATGTGTGCTATTTTAGGTGTTTGCCAATTATTTAAAAGCAGACTGATAATA
TAACTACTTTGGGATAGATAAAGGTAACAAAAATCTGAGAAAAAGGAACTAAGAAATTTGATGTTTTAAGTTCAGT
TAATATAAACTCACAGGACTACCTGTGGTAAGGGCTACAGAAAGATTAGACAATGATTCCTGTCTCCATGATCTTA
TAATTATTGTGTACTAGACATCCTTATCTTAAAGTCTAAAAGTTATTCATAGTAGAGCAGGCTTATTTTTTAAGAT
TTTATTTATTTATGAGAGACACACAGAGAGGCAGAGACACAGGCAGAAGGAGAAGCAGGCTCCCTGCAAAAGCCTG
AGGTGGGACTCGATCTCGGATCCTGGGATCACGTCCTAAGCTGAAGACAGAAGCTCAACCACTGAGCCACCCAGGC
ATCCCTAGAGCAGGCATATCATGTCTGTGATGTGAATACTTGCCTCTCATTTTTCCATCTACAAATGCCAACTGGC
TATGATGGTCATGCACCTTATTTTCCACCCCCTTCTTGTTGTTTTTCAAAGACTTTGTCCTTTTGACTCTGGGAAC
TCTTACTTATTTGCCCACTTTCTTCTTTTGGCTTAATTCAAGAAATCTGCAATTGACAATTCTTCATCATACTCTG
CTTCTCCTATTTCTACAATATATGCAAGTAGATATTTAGGAATTGGCTCCAAAGCCAAGGATGTAGCATTATCATC
AAGTCAGGCCTGTGGCAAGAATTATTAACTTCCTAGGATCCCCCAGTTATCTTTATACTCCAGGATGTTGATTCAT
TTCTGTTTTTTCCTTGGTACTTCCATCATTTTTTTCATGTTTCATCATGTTTTATTTATAGAAGCAAGTGTGAGGA
GTTAAAAGCAACTGGTTCTCGAGCTTCTGGCAAAAGCTTGAGCTGAAATGTTAGCTCTGGGCTGTGCTTGTGGGTC
AGCTGACTCAGATGTTGCCAGGCCCTCTGGGGTCATGGCAAAGGCCACATGCCTTGTGAGACTCTCTACTTTCTTC
CTGGCATGACTTTCTTCCCCCCTTTCTTGCTTGCACCTCAATACCAGGGTCAGTCAGTGTGGTCTGGATCAGAGAT
GACCTTCCCAGGATAGTCAGGTCTGCAATTGTTCTCCTTACTTTTTCCAATAAAAATAGTAATTAGTTATTGGGAA
ACTTTGTAAAGATAATGCTTCTGTTATAGGTTCAGCTCCAAATCTTTTATTGCTAAGTGTTTTTACTCACTGTGTT
ATTACCAGGAGCTAATGCTGTCATTCACTGCCACCGTGCTCCCTTTCTAAATCCCTGGGTTTCCAGAAGTCATCAC
GAGTTATATTCAGAAAATTGTAGTGATAAAAATCTTCCATGTGTAAAAAAACTCATGGGAGGTGACACCCAAATAC
AAACCACAGTGCCAGGAATTCTAAGTTTCAGCTTGCCAAAAAGCCAGACACATGTTAGGGTTACTCAAAACGATGA
ACATGGATTTTAAAATTCCACGTTGACTAATCTAGGTAGATATGGTCACCAAATAGTCAAGTAAAAGATGTAATAA
TTCTGCATAACCTGCTTGTCTTTTGTACTCCTACCTTTGCTCTATCCTTTCCCCTTCCTCTTTTCCTCATCAATTT
TATTGAGAATGAGAATGTCAAAGTAGAGAAATTAAACTCAGGTTGTGAGAAAGCCAGGATAACAATACTTATATTG
GGTAAACTAGGCTTTAAAACAAAAACTGTAACAAGAGACAAAGAAGAACACTAAAGAATAATAAAGGGGGCAATCC
AACAAGAAGGTCTAACAGTTGTAATTATTTATGCTTCAAACATGGGAGCACCCAAATACACAAAACAGTTAATAAC
AAACATAAAGGAAATAATTGATGGTAATACAATAATAGTAGGGAACTTTAACACCTTACTTATAACAACAGATTGA
TAATCCAAACAGAAAATCAATAAAGAAACAATGGCTTTGAATGACACCCTGGACCAGATGGATTTAACAGACATAG
TCAGAACATTCCATCATAATACAGCAGAATACATTCTTTTCAAGTGCACATGGAGCATTCTCCAGAAAATGTCATA
TATTAGGTCACAAAATAAACCTCAACATAGTCAAAAAGATCAAAGTCATACCATGCATATTTTCTGATCACAATGC
AATAAAAATCTGGAAAGAGCACAAGTACATGGGTTAAATAACAGGCTGCTAAACAATAAGTGGATCAACCAGGAAT
CAAAGAAGAAATAAAAAATTACATGGGAACACATGAAAATCAAAACAAAATGTTCCCAAATCTTTGGGATGCAAGA
AAAATGATTCCAGCAGCGAAGTTTATAGCCATACAGGCCTACTTCAAGAAGCAAGAAAAATCTCAAATAAACAACC
TAACTTTACACCTAAAGGAGCTAGAAAAAGAGCAACAAACAAAACTGAAAGCCATCAGAAGGAAGGCAGTAATAAA
GATTAGAGCAGAAATAAATGATATAGAATCCTAAAAAACTCCACAAAAATAAAAACAAAAACCCAGTAGTTCAATG
AAACCAGGAGCTCGTTCTTCGAAAAGATCAACAAATCGATAAATCTCTAGCCAGACTCATAATAAAAAAAGAGAGA
AGACCCAAAATAAACAAAATCACAAATGAAAGAAGAGAAGTAACAGCCAATACCACAGAAATACAAACAACCTTAG
GGGAATATTATAAAAAAGCTATATGCCAATAAATTGCACAGCCTGGAAGAAATGGATAAATTCCTAAAAGCCTATA
ACCTCCCAAAAATGAATCAGGAAGAAATAGAAAATTTGAACAGACTGCCAGCAATGAAATTGAGTCAGTAATTGAA
AAACTCCCAACAAACAGAAGTCCATGGCCAGATGTTTTCACAGGCAAATTCTACCAAACACTAGAAGAAGAGTTAA
TAGTTATTCTTCTCAAATTATTCCAAAAAATAGAATAAGGAAAACCTCCAAATTCATTCTATGAGGCCAGCATTAC
CTTGATACCAAAGCCAGATACAGACTCCACTAAGAGAAAGAACCAAAGGCCAATATCCCTGATGAATGCAGATGTA
AAAATTCTCAATAAAATGTTGGCAAGCTGAATTCAACATTAAAAAAATGATTCACCAAAGCTGACAGTGAGAGCTG
AGTGTTTGCTGTTTTTACAACAGAATTCCACCCCGTGGCTCCATCAGAGATTTCTCAAAAGCCAGGTCTGCTCAGC
TAGGCCAGGGCAAAGCCTCTCACCCTTACTCCTTCACTCGCTAGGCACATATTCAAGACAAAGCAAAGCCTCAGTA
GAGCGGTTTTGTATTTCAGGGACTGTAGAATGCCTTGTCAGAAATTGTTTAAAAGGAAGATTTGGTGTCCATCGAA
AGATGAATGGATAAAGAAGATGTGGTCTATGTATACAATGGAATATTACTCAGCCATTAGAAATGACAAATACCAT
TTGTTTCAATGTAGATGGAACTGGAGGGTTCCATCAACATTGCTGAGTGGAATAAGTCAATCAGAGAAGGACAAAC
ATTATATGGTCTCATTCATTTGGGGAATATAAAAAATAGTGAAAGGGAATAAAGGGGAAAGAAGAAAAAATGAGTG
GGAAATATCAGAAAGGGAGAGAGAACATGAGAGACTCCTAACTCTGGGAAACGAACAAGGGGTGGTGGTATAAAGG
GAGGTGGGTGGGGGTGGGGGTGACTGGGTGATGGGCACTGAGATGGGCCCTTGAAGGGATGAGCACTGGGTGTTAT
TCTATATGTTGGCAAATTGAACACCAATAAAAAATTTTAAAAAATGATTCACCCCAATCAAGTAGGATTTATTCTC
AGGATGCAAGGGTGGTTCAATACCTGCAAATTAATCAACATGATACATTACATGAATAAAAGAAATGATAAAAACC
TTATGATCATCTCAATAGATGCAGAAAAAGCATTTGGCAAAGTATGACATGCATTCATGATAAAAACCTTCAACAA
AGTCGATTTAGAGGGACCAAGCCTCAATATAATAAAGACTTTATATGAAAAACCCACAGGTAACATCAGACTCAAT
GGTGAAAAATGTAAAGCTTTCCCCTAAAGATCAAGAACAAAACAAGGATGTCCACTCGTACCACTGTTGTTCAACA
CAGTACTGGAAGTCCTAGCCTCAGCAGTCAGACAACAAAGGAAATAAAAGTCATCCAAATTGGTAATGAAGAAGTA
AACTTTCACTGTTTGCAGAAGACATGATACTATAGATAGAAAATATTAAAGATTACACCAAAAAACCCATACTAGA
TCTGATGAATTCAGTAAGGTTGCAGGATACATAATCAATGTACAGAAATCTGTTGCATTTCTATGCATTTATAATG
AAGCAACAGAAAGAGAAATTAAGAAAACAATCCCATTTACAATTGCACCGAAATAATACATCTTCTAGGAATAAAC
TTAACGAAAGAGGTGAAAAACCTATACTCTGAAAACTATAAAACACTGATGAAAGAAATTCATCTTTCTAGATATG
TCTCCTGAGGCAGGGTAAATAAAAGCAAAATTAATCTATTGAGACTACATCAAAATAAAAATCTATACAGTGAAGG
AAACAATCAGCAAAACTAAAAGGCAACCTACAGAATGGGAGAAGATATTTGCAAATGAAATATCTGATAAAGGGTT
GGTATCCAAAATATATAAAGATCTTATATAACTCAGCAGCCAAAAATTGAATAATCCAATTAAAAAATGGGTGGAA
GACTTGAAATGACACTTTTCCAAAGAGGACATACACATGGCCAACAGGCACATGAAAAGATGCTCCACATCACTCA
CCATCAGGGAAATGCAAATCAAAACTACAGTGAGATCCCACCTCACACCTGTCAGAAATGTTAAAATTAACAACAC
AGGAAACAACAGATGTTCACAAGGATGAGGAGAAAGGGGAACCATCTTTCACTGTTGGTGGGAATGCAAACTGGTG
CACCCACTCTGGAAAACTGTGTGGAGGTTCCTCAAAAAATTAAAAATAGAACTACCTTATGACCCAGCAAATGTAC
TACTAGGTATTTACCCAAAGGAAAGAAAAATACTGATTCAAATGTTTATAATAGCAATGTCCATGATAGCCAAACT
ATGGAGAGAGCCCAGATTTCCATCAACAGATGAATGGATAAAGAAGCTGTGGTATAAATACAATGGAATATCACTT
CTGTCATAAAAAAAGAATGTGATCCTATTATTTGCAGTGATGTGGATGGAACTAGATGGTATTATGCTAAGCGAAA
TAAGTCAGAGAAAGACAAATACCATATGATTTCACTCACATCTGGAATTTAAGAAAGAAAACATATAAGCATATGG
GAAAGGAGTAGGAAAAAAAGGAGAGAGAGAAACAAAGCATAAGAGGCTCTTAGCAATAGGAAATAAATAGGAAACA
AATAGGAGGATTGCTTGAGGGGACCTGGGTGGGGGATGAGCTAGAAGGGGTATGGGCATTAAGGAGGGTGTTGTGA
TGATGAGCACTGGGTGTTACATATTAGTGATGATCACTGAATTCTACTCCTGAAACCAATATTGCACTGTATGTTA
ACTAAATAGAATTTAAATAAAAATTTGAAGAAAATAAAAATAAAAAATAAAAATAAATAAAAATTATGTTTTTGCA
ATCAAAAGAAAAGAAAATGTTATCTTTATACCTACTACCTCAAACACATTTTAAAGTAGATTTGGATAGATCATTA
TAGCATTTTGCCCCTACATTAGAGGTGTTTTATCTTAATATTTAACTCAATTTGGAATGTGGGCAGATGGAAGAAG
AGGACTCTCTAAGGTCAATATGAAGGATATGGAAGCTGGATCACTTTAAAGAACTGGTAGAGAATGTCAGAAGTTA
AGAAAAAAAGTGTCATCTGGTCAATACTGCCATAGCTGCTATGAAAAATGCTTTTGTTGGATAACTAGGAAGTCTT
GGTGATCTTAGGTAGCAAAGTTTCATGTTAAGGCTCAATTTAGAGTCCCTTGAAATCTCAGGCTATCTTCATTAAA
CTATGTCTTCAGCACATGCTCTTGTGCTCAGTCACAAAATTCATGAACATCCAAACAGACTGACCTGCTGAAGAAC
CCATAAAAAAGACTATAGAGAAGGTGAATAAATTGCAAAATTTTTGCCAGCAAAAGCCAGAAACACATTCCAAATA
ATACATGATAGTACTTATGTATGTCATGCGTATCATTGACTCAGTTTTTCTGGATATTATTTTGAATTTCTGTACA
TGTTCTCATGGAGAGAACACATTTTCCAGATACAGAGGTTCTGAAGGAGTACTCTGGAGATCTGTTCACCATGTAA
GTCACATAGAAACAGAAATAGTGGTTTTCATAGCAATTCAAACTGTCGGGTTAGAAAAAACTAACATGTAATTTTA
AAGGACACTAATACACATGAGTTTTGTTACAAAACGTAGACTGATGGGACTTTTGATTTCACTCTTCAAAGTATGG
AAAACTTTATTAATTTGGTTAACATATCCTCATCTCTTTGGACGGAAAAATCTGTAACAACTAAGAACCTCAAATA
CAGCATTTCATTGCCTCCTTGACTCATGTTTTGAAGATGTACTAATTGCTTCTTGTCCTTTACTTCTTTAAAAAAA
AAAAACCAAGGAGGGACGCTTGATGGAAAAAGAACAATTGGTAAGGTTATTAGTTGTGAATGACTTAGAGAAAAAC
AAAAACCAAAAGACGTTTGTGTTGTTTGATAATTTTCCCTAAATAACATTTTCTTGGTACATATATTTTTCTAATT
ATTTGTTTTGTTACATATTTTGAAAATATTTCAGCCTTTTGAAGGAAAATTAGAAAACATCTTGTTTTCTCAGATC
CAGGTATGTGAGTCTAAGATGCCTGGAATTTTTAATTAGCTGGAATGAAGGAAAAACATCGATATATAGTATGAAC
TTGCCTTTTAATGAAATTGATGACTTTTTTTTGCTTTGATTACATACTCATCATTTTATTATTAATTTTATTTTTA
GAAAATAGTGGGAGTTGGAAAAATATTAAGAATATGTTGACTTATGACTAAGGTAGTGGTCTGTGGGCCTATCTGA
CAAGGAAGATAATATCAGGGTGTTCCAGAATGCATCCTACTTGTCTAGCATTGACATAGGTACAAAAATAAAATGT
TCAAATAAGGCTTTTTTTTCCTTCAATAGCTTTTAATATTTAAATAGTACCATCCTACTATCATTTAAGAGTTTGA
GGTATTTATGTTATTTTATTAAAAAAAATAAATGTTAGGAATAAGTTCTCTAGAAATTGAGGAAAGGAGATCAGTG
TACATGGGAGTGTTTCTCAAACTTCATTAAGGAGGCTGATCTTGACTTGGCTCTAGCTTCCAGAGGGATGAGTAGA
AGGATTTAGTTGGTGGAAGGAGAAGTAGGGTGAAAACAGACACTGCTGACTCAGCACTCCTCTCTCACTCATGGCT
GCCCACAATGGGCAGCTGGTAGATGTCCCGTCAAATTAAAGTGGACAAAGTGTTTTACCACCATTTTCCTAAGCCT
TGATTGAAGGTTTTTGTTCCAAGTAGGTGGTGGGATTTTGAGTGTGGAATTTACGTAAGCTATTAAGAGAATAGTG
TAAAACAGGTAGAAGCCCATTCTATTAAAATTTCTTTTGCTCTTCAGGTGTGTGCTATTTTCATAAACGTTGCTCA
CATCTATTACAAACATTTCTAAACACATATTTAAATATATTTTATATAAATAAATATGGTCAAGAAATCATCATGA
ATGTTATTATCTGGTGGCTGGTGATAATCCTTCTATCAGTTTTTTCCCCTTTGAAAATTCCTGTAAGTCAAAGATG
AAATTTTTAAAAACTATCAGAAAAAGTGATGTCAGCAAACAAAATGGACTGGATAATTCCAAAGGCCCATCCCTCA
GAGAAATACTAAAATATGAAGCAAAAACTGTCAGAAGCAACTTTGTAAGAACTCTGGAAAATAGCCAAAAGTTTAC
AGAAAGCAAGAAAATGCTGGATAAGAAAGGAGAGATAATGGTGGTGACAGTTTTAACATAATAGGAAAGCTTTGTC
AAATTTTGCTTGCTCTTGCCTCACTTGCCTTCCTGGTCTGGTGGTGATCTTGAAGATAGTAGCCTGAGTTCCCAGT
GTGGACTCTGGTCTCTGGTCCTGGAGGGAGCAGAAAAGATCATATTCTTAAAGAACTATGTTTTGTCTGTTTTGAT
CTATCTGGAGATGACCTAAAGGATGGATGTAAGGTGCTTGATTTTGTTCCACCCAACTTGGAACTCTCTTGGGATC
AAAGAGTGGCTATGCAGAGGGCATTCCTTGAAAACATTGTGAGGCAATAGAACAATGTGCTATAGCCTGGGGCAAA
AGACTGCATTTAAGCCAAAAAATGACACTCCAACAGTCCAAGAGTCCAGTTACTCCAAGAGAAACTGGGGAGAGAG
TTTCTTTGGGAGATTAAGCCATTGAGAAGTGCCTGTATAAACCAGAGAATCTAGAAAGCTGTGTGCATAGCCAGGA
CAGAATGCTTTCTCAGAGAGACCAGGCAACACCCAGAGCTTTCATCTATGGCCATTCTTTGGGCTCACAGAAAGCA
TAAAGTGGGGGCTAATGCAGACTTGTAAACAGCCTGGCTAAGCACTGAAAGAACCAATCTGCAAACATTGGGAGAG
GCTCTTTTTCTCCTCTCTTCCCTCTCCTTCCCTTCCCTTCCCTTCCCTTCCCTTCCCTTCCCTCTCCTTCCTTCCT
CTCCTCTCCTTTCCTTTCCTTTCCTCTCCTCCCCTCTTCTTTCTTTCTTTCTTTCTTTCTTTCTTTCTTTCTTTCT
TTCTTTCTTTCTTTCTTTCCTTCTTATTTTCTTTCTTCCTTCACCCAGGCATTCAAGGCAATCTCTGTCAACACTA
GCTGACTATAAGCCAAAGGTCAGAAACTTCAGAAACTACATCCAACAAATAATACAGACTTTACAGAAATAGTTTA
GAAAAGTAACAAAAGAAACAAACCAGTACAGCCTACAGCAAGCAAAAATAAAATAAATTACCCAAGGAGGGGGAAG
AATCTGATTTCCAGTTATCATATTATAATATTCAAATATCAGTTTTCCACAAAAAATTACAATATTCAAAGAAAAA
AAGAAAGTAGAACCTATCCACAGAGGAAATTAACAGAAATTGTCATTTAGGAAGCACAGATATTGGACATATGAGA
CAAAGATTTTAAACCAACTGACCTACTGGACTAGCAGAGTTAAAGGAAACCATAGACAAAGAGGTAGATATCAGTA
AAATGAAGTCTCAACAAATAGAGAATATCAATAGAAATTATAAAAATGATCTCAACAGAAATGCTGGACCTGAAAA
GTACAATACCAGAAACGAAGAATTCAATAGTAGGTTTCAGTAGAATATTTGAGCAGGTAGAAGAAAGAACAAGCAA
ACTTGAAGACAGATCAATTAAAATTACCCAGTGTGGGGAGAAGAAAGAAGAGGGAATGGAGAAAAAGACATCATCA
AACATACAAACATACTCATCAGGTGAGTTCCAAGAAAGAGGACAGAGAGAAAGGGACAGAAAGAATATTTGAAATA
ATAATGGCCCCAAATTCCTCCAAGTTGAACATGAATCTACACATCTGAGAGGCTCAACAAACTTTAAGGAGTATAA
ACATGAGGATAACCATTCTGAGACATACTATAATTTAACTGTTAAAAATCAAAAATGGAGAATCTTGAAAGCAGTG
AGATAAGCAATGCATTATATATGAGTGATTCTACATGAAATTAACAGGTAATTTCCCAGCACAATCCATGGAATTA
GAAGGCATGGAATGACATATTAAAGTGTTGAAAGAAAAAAATCTGCCAACCAAAAATTGTATATCTGGCAAAACTA
TCCTTCAAATTGAAGGAAACTCTAGGGCATTTCCCAATAAAGAGAAGCTGAGGTAGTTCATCGCTAGTAGACCTGC
TCTATGGAAATACTAAAGGGAATCCTTCAGGTGGAAATGAAAGAACACCAGATGATGACTTGAAGGCATACAAAGA
AATGAGGAACACTGGGAAAGTTCACTAGATAGGTAAATACAAGCGATAGCATGCCCAGTTTTGCCACTTCTGTTTA
GCTTTATACCGATGTTCTAGCCAGAGCGATTAGGCAAGCAAAAGAAACAAAGACATCCAAATTGGAGAGGAATAAG
TAAAACTATTTATATCCACAGATGATCTTATATGTAGAAAGTCCGACAGAATCCACAAAATACAATTAGGGCTAAT
AAATTGTGAAAAACTGCAGGATATAAGATCAATACACAAAAGTCAGTCTTATTTTTATACACTAGCAATGAATTGT
CCACGAATAAAACCAAGAAGACTTACAATAACATCCATAATAGTGAAATACCTATAAATAAATGTAACAAAGGTGA
AAGACCTAAATAAATGGAGACAACCTGTGTTAATGGATTAGAAGACTTAATATTCATGTGATCTACAGATTTCAGT
GTGATCTGTATCAAAATTCCAATAGCCTTTGTCCCCCCAGAAATAGCCAATCCTCAAATTTATATGGTATTGGAAG
AAGTAAAAACAATTTTGAGAAGGAGCACAGTTGGAGGACTCTCACTTCCTGATTTCAAAGTTTAATAAAAAGCTAC
TTTAGTTAAAACAATGTGGTGCTGACCTAAAGAAAGACATAAAAATCTGTAGAATAGAATAGAAAGCCCAGAGATA
ATTCTTCACACATATGGTTAACTAATTTTCAACAAGAGTGCCAAGATCATCATACAGGGAATGAATAGTTTCCTCA
GCAGATGATACTGGGGCAAATGGACATGTTAAAGAATACAGTTGCACCCCTACCTCATACTATATACAAAATTAAC
TCATTATCTACATTTAGATAAGCAACCTAAATGTAGGAGCTAATGTTATAAAACCCTTAGAAGAAGAGGTGAATCT
TCATGACTGAGGATTTGGCAATGGATTATTAGATAATTCAAAAGCATAAATAATGAAAGAAAAAGCAAATGAACTA
AACACCACCAAAATTTAAAACGTTTGTGCATTGTAGGACATTGTAGAGAAAGTGAAAAGACAACCTACAGAATGTA
AGAAAATATTTGCAAAACAGATATTTGAACAGAATATATTCATTCAGAATATATAAAGAGCTCAACTCAACAACAA
AAAGGCACACAACCTGATTAAAAATGGACAAAGGAGCTGAGTAGACATATATTTGAGGAAAGTATACAAATGGCTA
ACAAGTATATAAAGTATGTTTAACATTATTAGGCATGAGGTAAATGCAAATTAAAACCATGATAGAGATCACTTTA
CACCCACTGGGATGTCTATAATAGAAAAAGTGTTGTCAAGGATGTGGAGAATTTGGAGCCCTTGCATACTGTTAGT
GAGAATGTAAAATGGTGCAGCCACTATGGAAAGTCATTTAGTGGTTCCTCAAAAAGTTAAACATAGAACTATCAAA
TAACCCAGCAATTTTACTTGTAGGCATACTCACCACCCCTGAAATAGAAAAGAGATACTCAAGAGGGCCTGGATGG
CTCAGTCAGTTAAGTGTCTGACTCTTGATTTTATTAGTTCAGGTCATGATCTCAGGGTCATGATATTGAGCCTTGC
AGTGGCTCCATGCTCTCTCTCCCTCTCTGTCCCTCTCCCTCCCCACCCCTGCTCACACTTGCATGTGCTCTGTGTC
TCTAAAAAAATCAAAATTTAAAAATTTAAGGAAAGAAAAGAGGTATCAAACAAATACCTGTACTGGGATCTTCACA
GCAGTACTTTTCACAATAGACAAAATGTGGATAAAACAAAATGCATTAATGGATGAAAGGACCACCAATGTGGTAG
GTACATAGGATTGGTTATTATTTGGCAATAAAAAGAATGAAGTATCTGTATATGTTATACTGTAAACATCTCAGAA
ACATTATGCTAAGTGCAAAAAGCTGGACACAAAAGGTCTTACAGTGTATGGCTCCATTTAAAAAAAAATTTTATTG
GAGTTCAATTTGCCAACATAGAGCATAACCCCCAGTGCTCATCCCGCCAAGTGCCCCCCTCAGTGCCCATCACCCA
GTTACCCCAACCCCTGCCAACCTCCCCTTCCACTACCCCTTGTTCGTTTTCCAGAGTTAGGTGTCATGTTTTGTCA
CCCTCACTGATACTTTCACTCATTTTCTCTCCTTTATTTAAAAAATAAATTTATTTTTTATAGGTGTGCAATTTGT
CAACATATAGAATAACACCCAGTGCTCATCCCATCAAGCGCCCACCTCAGTGCCCGCCACCCAGCCAGCCCCACCC
CCATCCCCCTCCCCTTCCACCACACCTAGTTCATTTCCCAGAGTTAGGAGTCTTTCATGTTCTGTCTCCCTTCCTG
GTATTTCCCACTTATTTTTTTCCTTTCCCCTTTATTCCCTTTCACTATTTTTTATATTCCCCAAATAAATGAGACC
ATATAATGTTTGTCCTTCTCCGATTCACTTATTTCACTCAGCATAATACCACCCAGTTCCATCCACATCAAAGCAA
ATGGTGGGTATTTGTCATTTCTAATGGCTGAGTAATATTCTATTGTATACATAAACCACATCTTCTTTATCCATTC
ATCTATCGATAGACACTGAGGCTCCTTCCACAGTTTGGCTGTTATGGACATTGCTGCTATAAACATCGGGGTGCAG
ATGTCCTGGCATTTCACTGCATCTGTATCTTTGGGGTAAATCCCCAGCAGTGCAATTGCTGGGTCATAGGGCAGGT
CTATTTTTAACTCTTTGAGGAACCTCCACACAGTTTTCCAGAGTGGCTGCACCAGTTCACATTCCCACCAACAGTG
CAAGAGGGTTCCCCTTTCTCCACATCCTCTCCAACATTTGTGGTTTCCTGCCTTGTTAATTTTCCCCATTCTCACT
GGTGTGAGGTGGTATCTCATTGTGGTTTTGATTTGTATTTCCCTGATGGCAAGTGATGCAGAGAATTTTCTCATGT
GCTTGTTGGCCATGTCTATGTCTTGCTCTGTGAGATTTCTGTTCATGTCTTTTGCCCATTTCATGATTGTATTGTT
TGTTTCTTTGCTGTTGAGTTTCGTAAATTCTTTATAGATCTTGGATACTAGCCCTTTACCTGATAGGTCATTTGCA
ACTATCTTCTCCCATTCTGTAGGTTGTCTTTTAGTTGTGTTGACTGTTTCTTTTGCTGTGCAGAAGCTTTTTATCT
TGATGAAGTCCCAAGAATTCATTTTTGCTTTTGTTTCCCTTGCCTTCATGGATTTATCTTGCAAGAAGTTGCTGTG
GCCAAGTTCAAAAAGGGTATTGCCTGTGTTTTCTAGGATTTTGATGGAATCCTGTCTCACATTTAGATCTCTCATC
CATTTTGAGTTTATCTTTGCGTATGGTATAGGACAATGGCCAGTTTCATTCTTCTGCATGTGAATGTCCAATTTTC
CCAGCACCATTTATTGAAGAGACTGTCTTTTTTCCAGTAGATGGTCTTTCCTGCTTTATCGAATATTAGTTAACCA
TAAAGTTGAGGGTCCACTTCTGGATTCTCTATTCTGTTCCATTGATCTATGTGTCTGTTTTTGTGCCAGTACCACA
CTGTCTTGATGACCACAGCTTTGTAGTACATCCTGTAATCTGGCATTGTGATGCCCCCAGATATGGTTTTCCTTTT
TAATATTCCCCTGGCTATTCGGGGTCTTTTCTGATTCCACACAAATCTCCAGATGATTTGTTCCAACTCTCTGAAG
AAAGTCCATGGTATTCTGATAGGGATTGCATTAAATGTGTAAATTGCCTTGGGTAGCATTGACATTTTCACAATAT
TAATTCTTCCAATCCATGAGCATGGAATATTTTCACATCTCTTTCTGTCTTCCTCAATTTCTTTCAGAAGTATTCT
GTAGTTCTTAGGGTATAAATCCTTTACCTCTTTGGTTAGGTTTATTCCTAGGTATCTTATGCTTTTGGGTGTAATT
GTAAGTGGGATTGACTCCTTAATTTCTTTTTCTTCAGTCTCATTGTTAGTGTAGAGAAATGTCACTGACTTTTGGG
AATTTATTTTGTATCCTGCCGCACTGCCAAATTGCTGTATGAGTTCTAGCAATCCTGGGGTGGAGTCTTTGGGTTT
TCTATGTACAGATTCATGTCATCTGCAAAGAGGGAGAGTTTGACTTCTTCTTTGCCAATTTGAATGCTTTTTATTT
CTTTTTGTTGTCTGATTGCTGAGGCTAGGACTTCTAGTACTTTTTTGAAGAGCAGTGATGAGAGTGGGCATCCCTG
TCATGTCCCTGATCTTAGGGGAAAGGCTCCCAGTGTTTCTCCATTGAGAATTATATTTGCTGTGGGCTTTTCGTAG
ATGGCTTTTAAGATGCTGAGGAATGTTCCCTCTATCTCTACACTCTGAAGAGTTTTGATCAGGAACGGATGCTGTA
TTTTGTCAAATGCTTTCTCTGCATCTATTGAGAGGATCATATGGTTCTTGTTTTTTCTCTTGCTGATATGATCAAT
CACATTGATTGCTTTACAAGTGTTGAACCAGCCTTGCATCCCGGGGATAAATCCTACTTGGTCATGATGAATAATC
TTCTTAATGTACTGTTGGATCCTATTGGCTAGTATCTTGTTGAGAATTTTTGCATCTGTGTTCATCAGGGATATTG
GTCTATAATTCTCCTTTTTTGGTGGGGTCTTTGGTTTTGGAATCAAGGTGATGCTGGTTATGGCTCCATTTATATG
AAATACACAGAATTGGTAAATTAACGGTTACAAAATCAGATTGGCAGGGTGTCAGGGGCTGGGATGAGGGAGAACA
CAGAGTGCCTGCCTACTATCACAAGGTTTCCTTTTTGGCTCACAAACTGTTTTGGAATTTGATATGGATAGTCGCA
CAATGTTATGAATATATTAAATGCCATTCACTTTAAAATGGTTTATTTTACGTTATGAAAATTTCACCTCAATAAG
AAATCACTGGACCCTGTGTTCCAGGCAAAACAGAAAGAGAAAATAGAGAAATGAATTATTTTGATTAGCACTGTAA
TTAAAATTACGCTACTCAGATAAGGGCACATTCAGTCATTCTGTAATTTATTTCTTACAACTTCACTGAATGTCTA
ATTATCTTGAAATAAAAAAATTAATTGAAAAGATTATTGGAGTTTGTTGAAAAGCTGAAAGGTCGTTTAAATGGGG
AGATATAATCAATAGGTACTTGGGACTTAGTCCACTCTTCTATTTTCTATTAGTTTAATAATAGACACATACTACT
TACTGTGTGGTAGGCACAGTGCTAAGTGCTTTACAAATAATAACTCTGTAAGCAGCTATTCTTATAGCCACTTCCT
GGAACAGTGTCCCCAGTGCTGGCAACAGTGCTCGGCACTTGGTAAATGCTACATAGATAAGTGTTGGAGGATTAGA
TGGTACTCTTTCCAAGTTGCCTCATTGCCTTTGTTTCAGTATCTAAATTCCTAGATAAAATTTCTTTCATCTAGAT
GACTCTAGATCTTCAGATATTTGGCTTCTTCTGAAAGTAGAACTTTATATGTGAAGCTGGGAGCAGATGCTTTGAT
TGGGAGGGGAGCCCAGGAAGCACTAGAGAAGGTGCATAGATAATGGGCTGGGGAGGGAGAAGAAGCCCACACTATG
GTAAGTCCTTGAAGTTGAGGCTGGCACAGTTTTTCAAGAATCTCTGAGGAACATACAGAATGGGACTCTCCATCTG
AAGTACAGGAGGTTGGGGCACTTCTCTGCCACTTCCCATGCCTCCCTGCCTGAGAGCTGCCCATACTCCTGGACTT
TGCTTTTGTGGGGCTGAGCAAGCTCTTTGGCTCTGAACAGGCTTTGGTGTGGAACTGTGGAGGTGGAGTGCTTTAG
ATGGCATGTTGGCGCTTTTACAGACAGGTGGTCCCAACTGCAGCGGGAATCAGCTGCGGTGTGTTGATGAGATGCA
AGGCAGCAGAAGCATTTGCTACATCTGATAGATATGATCTTTAATTTAGTGAACAACTTTTAAATGATAATCAAAG
AAAATATCATAAATTAAATGCACAAATGTAACAGTTACAAACTGAGTTGTTAGATATCTAAAAGCCAGCATTCATT
TTCAGAAGATAGATCACTGTCTCAATGTGCTGTAGAAATATTTATGGTGATAGACATGATATGTACAAACTGTCTA
CCCTTAGGTCATGGAAACCTTCTGCTGTTGGCTTTCCCTTCCACCCACTCCCCCAGACTCAAGCTCTTTGGTTTAC
AGCTGACTCTTCTCAAGCATCTGTGTTTACTGAACAGCAGTTCAATATTCCCATAAATGGTCTGTGCTTCTCTAGA
AAAATGCTTGTTTTTCAGCATCATCACTGGTGTGTATTGTGATTGCTCAAAATTTAATGGAAGGGTTATGACTTCG
GTTTATACTCTGGTATCATTAGTGGGCCATGACCCACCTGGAGTTTGTAATACAGCATTTTCTCAGAAATCAGTCA
CAGGACCTGGGATTATTAAAAGAGAATGAAAACACATCTTTATCAAAGACCATATCGGTGCAGATGGAAAGCTATG
TCTATTTTTGAACATTAGCTTTTTTTCACTAATTTTTAATCGCAGACTACTTTTCCCTGAATATAACAAGTAGAAA
GTAGTTAATTTTCCTGTAGAAAGGTCTCTGAAGATTTCACTCATCCTTTCTTGTCTCTCTAATCTCCCAAATGACT
GGAAATAATGAAGGAAGAGATGCTGGTACTCCTCTGGGGTAATGATCTCATGACTTCCAAGGGTGGGTTTGCCGTC
ATAATAGTTGGGGACAAAAGTCAGGGTGGTAACTGGGGGTCGAATAAGATCTGGCAATCTTGAGAATGTGAAGTAA
TTTGGAATATAGCATGCTCACAGAGTGGGTATTTTATCCTGTTGGGTAGAAAAGACCATCAGGTGTGCTGGTGTCT
GTTACTCTTGGGAGCCCCTAGTTTAAAGACTAGGAGGCTAGCTCAGAGTTGAGGAGATGCCTTTTTTTCAAGAGTA
AGAATAATTTTGATTTTCAGTATTTCCAGTGGAGCAGATCCCTCCAGAAAAGTCGTGAGAGCTGCATGTCAATTGC
TGTTATCCCAATGGTATTTAGAACATTTTCTTGATTAATTACAAGAAAATTTGGCCTGGGAGGGGGCTGCATCCTG
CTGCTTTCAGGAGCCATGGCCTGGGAGCACAATTCCAGCAGTGCAGGCCCTGGATCCCAGGGTGCTAAGGGGACAC
AGCCCAGGATCCTGCACTCCTCTGGGGATAGGCAGAGGCAGGGAGAGCACAGGACAGTGAGGGCTTTCCTGCTGCT
GGGTGCCCCCAAGTTGTGCAGGTCAGTGACCCCCGCCCTGGGAGCATCCAGGCCAGTGCAGACTGGGAGACTGCGG
TAGTCACCGCAGGGAGCAGACTACAGGGCTAGGGACCTGGCCGCCACCAGTGGTGTTGTTCTTCTTTGTTTCACCC
TGTGCCTGGGAGAGGCAGGGTGTCAGGGAACAGGGGTCTCACGAGGTAAACAGCTCCCACTGAGCCCGGCACCTAG
CAGGGGGCACGGTAGCTCCCAGGTACACACACCTGAGAACCAGCACAACAGGCCCCTCCCCCAGAAGACCAGCTGG
ATGGACAGGGGAAGAGCAAGTTCCTGACTAAGCAGCACTAGGAAGGTCCAGGGGAAGTTGAGGGATTTACAGTACA
TAGAACCAGAGGTTACCCCTCCTTTTTTCCTCTTTTTTTCTTCCTTTTTCCAGTACAACTTGTTTCTATATCAGAC
TGTAAATTTCCATTTTTTTTCTTTTTTCCCACCTTAACTACAATATTTTACCACCTATTCATTTTTAAGATTCTTC
CTTTTTGACTTCAATATTTCTACAATTACAGGTCCTAGATATATTTTCCACTTCTAGATTCCCTTCAACGTGCTCA
CCATAATTTTGGGAGATATACAAGATATATTTTTTGTTTTGTTTTGTGTTTTGTGTTTTCTCTGCCTCATGTTGTT
CTACAATGGCAGAAGTTTTTTTTTAATAATAATAAATTTATTTTTTATTGGTGTTCAATTTGCCAACATACAGAAT
AACACCCAGTGCTCATCCTGTCAAGTGCCCCCCTCAGTGCCCATCACCCATTCACCCCCATCCCCCGCCCTTCTCC
CCTTCCATCACCCCTGGTTCGTTTCCCAGAGTTAGGAGTCTTTATGTTCTGTCTCCCTTTCTGACATTAGAAATGA
CAAATACCGACCATTTGCTTCAACGTGGATGGAAATGGAAGGTATTATGCTGAGTGAAGTAAGTCAATCGGAGAAG
GACAAACATTGTATGTTCTCACTCATTTGGGGAATATAAATAATAGTGAAAGGGAATATAAGGGAAGGGAGAAGAA
CTGTGTGGGAAATACAATGGCAGAAGTTAATACCTTCAAAAACATGACCAGTATGCACCCAGAACCAAGTGGTATA
CTGTGCTGGTTCATTCTGTGAGATTCCTCATTCCCATTCTGCCCCCCTGTTTTATCTCATTTATGTTTTGGTGGTC
AATGTTGGGGCCTTCTACAAGTATTTCTGTTTTATATAAATTTGGAACTGAGTGTCTTCTAATATACAGAACTTAA
TATACTCAGAAACAAGAGGATCACCCTCTAGAACCCCCCAGGTAGACTACATTCTCCCACTACTACAACTTCGTCA
CCACCACCATCTCCCAGTACCCCCCCCCCTTGAATTCTTCTCCTTTTTTTCTTTTTTCTTTTTCTTTTCTTTTTTT
TTCTATTCTTTAGGATTCCTGGCCTTTTATTTTTTACTACTTTGTTTTATAATTAGGTTTCACTTTAGTGGTCCTT
TTGTTTTATTTCATTCTGATCTTTGTTTTCAATTTCTGGTCTCTGACCCTGGCAGAATCATCTAGGGTGAAATTTA
CTTAGGTCATGGTTGATATTCTTGATGCAGCCCACTCATACAGCCATTCTGCACTGAGCAAAGTGACTAGAAAGAA
CTCACCACAAAAGAAAGAATCAGAAATAGTACTCTCTGCCACAGAGTTGCAGAATTTGGATTACAATTCAACGTCA
GAAAACCAATTCTGAAGCACAATTATAAAGCTACTGGTGGCTATGGAAAAAAGCATAAAAGAATCAAGAGACTTCA
TGACTGCAGAATTTAGATCTAATCAGGCCAAAATTAAAGATCAATTAAATGAGATGCAATCCAAACTGGAGGTCCT
AACGATGAAGGTTAATGAGGTAGAAGGAGTGAGTGACATAGAAAACAAGTTGATGGCAAGGAAGGAAACTGAGGGA
AAAAGAGAAAAACAAGAGACTATGAAGTAAGGTTAAGGGAAATAAATGACAGCCTCAGAAGGAAAAAAATCTACAT
ATCATTGGGGTTCCAGAGGGCGCCGAAAGAGACAGGGGACCAGAAAGTGTATTTGAACAAATCATAGCTGAGAACT
TCCCTAATTAGGGGAGGGAAACAGGCATTCAGATCCAGGAAATAGAGAGGTCCCCCCCTAAAATCATTAAAAACTG
TTCAACACCTTGACATGTAACAGTGAAACTTGCATATTCCAAAGATAAAGAAAAAACCCTTAAAGTGGCAAGAGAC
AAGCGATCCCTAACTTACATGGGGAGGGATATTAGATTAACAGCGGACCTTTCCACAGAGACCTGGCAGGCCAGAA
AGGACTGGAATGATATATTCAGGGTACTAAATGAAAAGAACAGGCACCCAAGAATACTTTATCTAGCAGGGCTCTC
ATTCAGAATAGAAGGGGAGATAAAGAGCTTCCAAGATAGGCAGAAACTGAAAGAATATGTGACCACCAAACCAGCT
CTGCAAGAAAGATTAAGGGGGATTCTGTAAAAGAAAGAAGTCCAAAGAAATAATCCACAAAAACACAGACTGAATA
GGTATTATAATGACACAAAATTCATATCTTTCAGTAGTAACTCTGAACGTGAATGAGCTAAATGATCCCATCACAA
GACACAGGGTTTCATACTGGATAAAAAAGCAAGACCCATCTATTTGCTGTATACAAGAGACTCATTTTAGACAGAA
GGGCACCTAAAGCCTGAAAATAAAAGGTTGGAGAACCATTTACCATTCAAATGGTCCTCAAAAAAATGCTGGGGTA
GCAATCCTTATATCAGATAAATTAAAGTTTATCCCAAAGACTGTAGTAAGAGATGAAGAGGAACACTATATCGTAC
TTAAAGGGTCTATCCAACAAGAGGACCTAACAATCCTCAATATATATGCCCCGAATGCAGGAGCTGCCAAGTATAT
TAATCAATTAATAACCAAAGTTAAGCCATACTTAAATAACAATACACTTATACTTGGAGACTTGAACATGGCACTT
TCTCTAATTAATCTTCAAAACACAACATCTCCAAAGAAACAAGAACTTTAAATGATACACTGGACCAGATGGATTT
CACAGATATCTACAGAACTTTACATCCAAACGCAACTGAATACACATTCTTCTCAAGTGTACATGGAACTTTCTTC
AGAATAGACCACATACTGGGTCACAAATCACGTCTTAACCAATACTAAAAGATTGGGATTGTCCCCTGCATATTTT
CAGACCACAATGCTTTGAAACTTGAACTTAATCACAAGAAGAAATTTGGAAGAAACTCAAACATGGGAAGGTTAGG
AGCATCCTTCTAAAAGATGAAAGGGTCAACCAAGAAATTAGAGAAGAATTAAAAGATTCACAGAAAGTAATGAAAA
TGAAGAAACAACTATTCAAAATATTTGGGATACAGCAACAACAGTCCTAAGAGGGAAATACATCGCATTACAGCAT
CCCTCAAAATTGGGAAAACTCAAATACACAAGCTAACCTCACACCTAAGGAACTGGAGAAAGAACAGCAGATAACA
CCTATGCCAAGCAGAAGAGAGTTTATAAATAGTCGAGCAGAACTCAATGAAATAGAGGCCCGAAGAACTGTAGAAC
AGATCAACAAAACCAGGAGTTGGTTCTTTGAAAGAATTAATAAGATAGATAAACCATTAGCCAGCCTTATTAAAAA
TGAAAAAGAAAAGACTCAAATTAATAAAATCATGAATGAAGAAGGAGAGATCACAACCAATACCAAGGAAATACAA
ATTATTTAAAAAACATATTATAAGCAGCTACACACCAATAAATTAGGCAATCTAGAAGAAATGAATGCATTTCTGG
AAAACCACAAATTACCAAACCTGGAACAGGAAGAAATAGAAAACCTGAACAGGCCTATAACCAAGGAGGAAATTGA
AGCAGTCATCAAAAACCTCCCAAGACACAAAAGTCCAGGGCCAGATGGGTTTGCAGGCAGATTCTATCAAACATTT
AAAGAAGAAACAATACCTATTCTACTAAAGCTGTTCTGAAAGATAGAAAGGGATGGAATACTTCCAAACTCATTTT
ATGAGGCCAGCATCACCTTAATTCCAAAACCAGACAAAGACCCCACCAAAAAGGAGAATTATAGACCAATTTCCCT
GATGAACACAGATGCAAAAATTCTCAACAAGATACTAGCCAATAGGATCCAACAGTACATTAAGAAGATTATTCAC
GATGACCAAGTGGGATTTATCCTCAGGATGCAAGGCTGGTTCAACACTCGTAAAGCAATCAATGATATAGATCAAA
TAAACAAGAGAAAAATAAGAACCATATGATCCTCTCAATAGATGCAGAGAAAGCATTTGACAAAATACAGCATCCA
TTCCTGATCAAAACTCTTCAGAATGTAGGGAATAGAGGGAACATATCTCAGCATCTTAAAAGCCATTTACGAAAAG
CCCACAACAAATATAATTCTCAATGGGGAAACACTGGGAGCCTTTCCCTTATGATCAGGAATACAACAGGGAGGTC
CACTCTCACCACTGTTATTCAACATAGTACTAGAAGTGCTCGCCTCAGCAATACTGTCTTGAGGATCACAGATTTG
TAGTATAACTTGAAATCCAGCATTGTGATGCCCCCGGCTCTGGTTTTCTTTTTCAATATTCCCCTGGCTATTCGCG
TTTTTTTCTGATTTCACACAAATCTTGAGATTATTCATTCCAACTCTATGAAGAAAGTCCATGGTATTTTGATAGG
GCTTGCATTGAATGTATACATTGCCCTGGGTAGCATTGACATTTTCACAATATTAATTCTGCCAATCCATGAGCAT
GGAATATTTTTCCATCTCTTTGAGTCTTCCTCAATTTCTTTCAGAAGTGTTCTATAGTTTTTAGGGTAGATCCTTT
ACCTCTTTGGTTAGGTTTATTCCTAGGTATCCTATGCTTTTGGGTGCAATTGTAAATGGGATTGACTCCTTAATTT
CTCTTTCTTCAGTCTCATTGTTAGTGTAGAGAAATGCCACTGACTTCTGGTCATTGATTTTGTATCCTGCCACACT
GCCAAATTGCTGTATGAGTTTTAGCTATCGATGGGGTGGAGTCTTTTGGGTTTTCTACATACAGTATCACGTCATC
TTCAAGGAGGGAGAGTTTGACTTCTTCTTTGCCAATTTGAATGCCTTTTATTTCTTTTTGTTGCCTGATTGGTATT
GGCACAAAAACAAACATGGATCAATGGAACAGAATAGAGAACCCAGAAATGGGCCCTCAACTCTATGGTCAACTGT
TATTCGACAAGCAGGAAAGACTATCTGCTGGAAAAAGGACAGTCTCTTCAATAAACGGTACTGGGAAATTTGGACA
TCCACATGCTGAAGAAGGAAACTAAAGCATTCTCTTATACCATAGACAAAGATAAGCTCAAAATGGATGAAAGATC
TAAATGTGAGACAGGAATACATTAAAATGCTAGAGGAGAACACAGGCAACACCCTTTGTGAACTTGGCCTCAGTAA
CTTCTTGCAAGATACATCCTGAAGGCAAGGGAAACTGAAGCAAAAATGAACTTGGGACTTAATCAGGATGAAAAGC
TTCTGCACAGCAAAAGAAACAGTCAACACAACTAAAAGACAATCTACAGAATGGGAGAAGATAGTTGCAAATGACC
TATCAGTTAAAGCGCTAGTATCCAAAGATCTATAAAGAACTTATGAAACTCAACAGCAAAGAAACAAACAATCCAA
TTATGAAATGGGCAAAAGACATGAACAGAAATTTCACCAAAGAAGTCATACACATGGCCAGCAAGCACATGAGAAA
ATGTTCCACATCAGTTGCCATCAGGGAAATACAAATCAAAACCACAATGAGATCCCACCTCACACCAGTGAGAATG
GCGAAAATTAACAAGACAGGAAACAACAAATGTTGGAGAGGATGTAGAGAAAGGGGAACCCTCTTGCACTGTTAGT
GGGAATGTGATCTGGTGCAGCCACTCTGGAAAACTGTGTGGAGGTTCTTCAAAGAGTTAAAAGTTGAGCTACCCTT
CAATCCAGCAATTGCACTGCTGGCGATTTACCCCAAAGATACAGATGCAGTGAAACGCCAGGACATCTGCACCCCG
ATGTTTATAGCAGCAATGTCCACAATAGCCAAACTGTGGAAGGAGCCTCAGTGTCTATCGATAGGTGAATGGATAA
AGAAGATATGGTATATATATATAAAATATTAATATATTATGAAATATTATATTTATATATAATATTTTATATAGTA
AAATATATGAATAATGAAATATTCATAATTAATAATTAATAATAATGATAAATTTTCATTTCAACAATGAAATAAA
AAAATGAAATATTATATATATACATATATATAAAATGAAATATTACTCGGCCATTAGAAATGACAAATACCTACCA
TTTGCTTCAACGTGGTTGGAACTGGAAGGTATTATGCTGAGTTAAGTAAGTCAATCAGAGAAAGACAAACATTATA
TGATCTCATTCATTTGTGGAATATAAAAAATAGTGAAAGGGGAAAGGAGAGAAAATGAGTGGGAAATATCAGAGTG
ACAGAACATGAGAGACTCCTATCTCTGGGAAACAAACAAGGGGTAATGGAAGATAAAGTGGACAGGGGATGTGGTG
ACTGGGTGACGGGCACTCAGTGGGGGCACTTGATAGGATGAGCACTGGGTGTTATGCTATATGTTGGCAAATTGAA
CTCCAATAAAAAATATACCAAAAAAATAAGAAGAAAATTTGGCCTGTTAGGGTCAAGTGGACCCATAGGGACACAG
AAGCATTCTAGACCATCATTAAAGGATTAAGAAAAAAGCAAACGAGTGAGATCAGCAGTTACTGAAGCCTCTTTTC
TCAAACTGAAGTGGCAGGATCAGATCCAGCTTCTTCTTCTTTTTAAAAATAACTTTGTTGGGATGCTCTCACTTAT
GTGTGGAATCTAAAATAGTAATATCCCAAGAAGCAGAGATAGAATGGTGGTTGCTGGGGGTGCAACTCAGGGAATA
AGGAGACTATCAAATGGTACAAAAGTTCAGTTATGCAAGACACACGAGTTCTGAAGATCTCATGAGTGCCATGATG
ATCATAGTTAACAATACTGTATGCTAAAAATCTGCTAAGACACAGATTGTGCTTCTTGAACTTGACTTTCCAGACT
CAACTGGCTGAGGCCAGTGAGGCACAGTCTTCCTTATATCAATTCCCAGCTGGCCCCTCCAGTCCCTGACTCCCTC
CCCCTTGCTTCTGGGCTTTAGTGAAGTTGTCTGGTTTCACCTGTTACCTGAAACGGCCTCCATGTCCCCTGACATC
ATCCAGGAGATGAGGGGCCCCAGTGGACCACACATGTGGATAATAAACACAATAGTCCTCCATGTCTTCTTCTAGT
TTGGAAGTTCTTATCCTCAGAGACACACTTCACTGCTTCCATTGTATGTAAAGTCTTACATCCAAACAGTAAAGTG
ACCCTGGCATGAGAACACCACTTTCTAGGGAATAACCACATGGTTGAGGAGCTTAGATCGATTCCCCCCAGTTCAG
CCCTATTCCATTCTGGATGTTCTCTGGGATGGCTTTAGTCTTCCAGTGCCATGCCCCTGCATGGACCTCCTTCCAA
AGGCGAGGTGTAATGGTCTGTCACTCTAGGCATTGCCTTATGTCTTCTGGTAAAGAATCTTGGTTGTGACAGCCAA
AAAGCAGGAATATTCCCATGAAATACTGCAGACACAGTGAAGAGAGACATTCTTTTTTGAGGAACTTAGTCTGGTC
TTTAGTTCACTTATAATTAAGATGCTGAAGAAAATGTATAATGTATTTTATCTACCCTGCACAAGATCGCCAAAAT
GTACTCTCGTATATCTCTCCACCCTGTCTCATCTTGTGATACAGTGAAGCTAACAGCAGTGATAGATAATATTTAT
TGAGTGCATTTTATTTTTCAGGCACTATGCTAAATACTTCACAGAATCTCATTTAATTACCAAACTATTCCCATGT
GTTAGATTATTATCATCCCCTTAACCTGAGCTGTGCTTTCTTTCCCACTGTCACTCAGCTAGTAAGGTGATAGAAT
TTGAAGTCACTTCTGACCCCAAGGCCACAGTTATTTACACTCTCAATGGGAAGGCATGCACTATAATGTGGTGATT
AACTCAAGTCACATGAAAAAACATTTATTAAGTGTTTGTGGTGTGAAATTCTGTGCTAAGTGCTGTGATCTGAAAC
AGCAGAAAGACAGTGTGCTAGCTGGCTAAGGCGGCTATAACCAAGTGCCATGGCCATGGGAGCTTGAGCAATAGAA
ATGCATTTCCTCACCACTCTGGAGGCTGGAAGTCTGAGATTGAGGTGTTGGCAGGGCTCCTTCGGTGGTGGGATCT
GTCCCAGGTCCCTCTCCTTGGCTTGAACATGGACATTTTCTCTTATTTTCCACATTGCTACCCTCTGCACATGTCT
GTCTAAATTTCCTGATCTTATAAAGATGCTTGTCTTATTGATTCAGGGCTGGGATAAAGCAGACCAAATGTGAGTG
GGATGTAGGGAAGGACAGCCCATGTGGGGTCTCTGATGATGCTAATGATTTTGTGTTTATCCAACAACCAAATGGT
TTCAAGTGCATCACGAAGCACATGAATTTTCATTCCCACCCATGCTTTTGCCATAGGTATGACAAGAACAGATGTG
AATTTTGGAAAGGTGACTGGCTCCAGGGTGATGAACTAGTTGAAGGGAAGCCACACTCCTGTCAGAAGACCAGCTG
AAGTTACTGCAGTGGAGGAAGGTCAGAGCCCGTGGAGGCAG (SEQ ID NO: 1)
TGTGCATGGTTGGTCCAGATTTGGGGTGTACGTGACTACCACTGTTTGTGTTATACATGATCA
GGAGAATGAACAGGAAGAATATGGAATCCACTTAACACATGTGGTGTCGCCAGGAGGAGCATG
ATTCTGCCAGCATTGACTAGACTTATCTCTGATATGTTTTCAATCTGCAAAACTGAGAAGCTA
GTAAATTTGTATCAATTTAGACTCTTTTCTTCAATGAGAAGTGAGTTGAAGAAGTGGCACTCA
GAAATTACCTTTGAGTTATCCAGCTGCTGGAAATCTAAGAGCTGTCCAGAGGCAAGAAATACT
TAAAACTTAGAGTGGTTAAGGTATGACAAGAACAGATGTGAATTTTGGAAAGGTGACTGGCTC
CAGGGTGATGAACTAGTTGAAGGGAAGCCACACTCCTGTCAGAAGACCAGCTGAAGTTACTGC
AGTGGAGGAAGGTCAGAGCCCGTGGAGGCAG (SEQ ID NO: 2)

### Example 3

Dogs can serve as excellent model of human complex disease, as many canine diseases including cancer show similar clinical and molecular profiles to their human correlate. Dogs receive modern health care, have recorded family structures, and largely share the human environment. In addition, based on the recent breed creation, purebred dogs have megabase-sized haplotypes and linkage-disequilibrium (LD), allowing genome-wide association studies (GWAS) in dogs to be performed with 10-fold fewer SNPs than in humans (Lindblad-Toh, Wade et al. 2005). Power calculations and proof of principle studies have shown that 100-300 cases and 100-300 controls can suffice to map risk factors contributing a 2-5 fold increased risk (Lindblad-Toh, Wade et al. 2005).

Golden retrievers, one of the most popular family-owned dog breeds in the U.S., have a high prevalence of cancer with over 60% eventually dying from cancer (Glickman 2000). Two of the most common cancer types in golden retrievers are lymphoma and hemangiosarcoma with a lifetime risk of 13% and 20%, respectively (Glickman 2000). Canine lymphoma and hemangiosarcoma are clinically and histologically similar to human Non-Hodgkin Lymphoma (NHL) and visceral angiosarcoma, respectively (Priester 1976; Paoloni and Khanna 2007).

Approximately 50% of lymphoma in the golden retriever is of B-cell origin, within which the most common subtype is diffuse large B-cell lymphoma (DLBCL) (Modiano, Breen et al. 2005). In human adults, DLBCL and follicular lymphoma (FL) are the two most common subtypes of NHL, accounting for 60% of all B-cell NHL in North America (Anderson, Armitage et al. 1998

In contrast, while canine hemangiosarcoma is relatively common, angiosarcoma is rare in humans, accounting for 2-3% of adult sarcomas (Penel, Marreaud et al. 2011). The rarity of this disease in human limits the feasibility of genetic studies. Angiosarcoma is a very aggressive cancer in both species where the angiogenesis caused by the tumor is accompanied by highly invasive and metastatic nature.

As described herein, a GWAS of B-cell lymphoma and hemangiosarcoma in 373 golden retrievers from the U.S. was performed. The study revealed two major loci on chromosome 5 that together explain approximately half of the disease risk in this cohort. In addition, RNA sequence analysis of differential gene expression in B-cell lymphoma identified that the risk alleles of those 2 loci significantly altered expressions of genes that affect T-cell mediated immune responses.

### Identification of Germ-Line Risk Factors

To search for inherited risk factors predisposing to B-cell lymphoma in golden retrievers, GWAS was performed using the canineHD Illumina 170 k SNP array (Vaysse, Ratnakumar et al. 2011) (FIG. 2A, Table 9). Since dog breeds contain high levels of cryptic relatedness and complex family structures, it was necessary to apply a method that could successfully control for the population stratification (Price, Zaitlen et al. 2010). This resulted in a final dataset of 42 cases and 153 controls, with 128,330 SNPs used for the association analysis. The quantile-quantile plot (QQ-plot) showed an inflation factor λ of 1.02, indicating that the population stratification had been well controlled (FIG. 2A). The plot revealed four SNPs with p-values below 1×10⁻⁵, at which the observed values significantly deviate from the expected distribution. Three of these SNPs were located on chromosome 5, while one SNP was located on chromosome 19 (FIG. 2A, Table 9). The top SNP on chromosome 5 (p-value=1.1×10⁻⁶) is located within the last intron of the STX8 gene, and had a strong allelic odds ratio (ORallele) of 7.0 by PLINK. When EMMA-X was used with a mixed model to calculate OR by a regression model taking other factors into account the ORreg=1.45. The top SNP on chromosome 19 (p-value=7.7×10⁻⁶, ORreg=2.12) was located in an intergenic region between the DPP10 and DDX18 genes.

An independent GWAS for hemangiosarcoma in golden retrievers identified significant association to 10 loci on six chromosomes (FIG. 2B, Table 9). After quality control, the dataset included 142 hemangiosarcoma cases and 188 controls, and 127,188 SNPs for the association analysis. The QQ-plot for this analysis showed that the observed p-values significantly deviated from the null expectation below 1×10-4 (λ=1.05, FIG. 2B), identifying 27 SNPs on six chromosomes as significantly associated. Of those 27 SNPs, 17 were located on chromosome 5. All but one of these 17 SNPs were located between 32.7 Mb and 37.1 Mb, overlapping with the region associated with B-cell lymphoma (Table 9). The top SNP (ORallele=2.50, ORreg=1.22, p-value=1.4×10⁻⁶) was located at 32,901,346 bp in close proximity to TRPC6 and in strong LD (r2>0.8) with 10 other significantly associated SNPs. The second most significant SNP (ORallele=2.75, ORreg=1.31, P=2.1×10⁻⁶) was located within the last intron of the STX8 gene at 36,848,237 bp, and a short distance (8.7 kb) away from the top SNP associated with the B-cell lymphoma (Table 9).

TABLE 9
List of significantly associated SNPs from each GWAS.

| | | | Alleles | | | | | | R² from | R² from |
|---|---|---|---|---|---|---|---|---|---|---|
| | | Position | (minor/ | MAF | MAF | OR | OR | | 32.9 Mb | 36.8Mb |
| SNP ID | Chr | (bp) | major) | cases | controls | (allelic) | (empirical) | P | top SNP | top SNP |
| B-cell lymphoma Analysis. Significantly associated SNPs (p-value <1.0 × 10⁻⁵) | | | | | | | | | | |
| BICF2G630183354 | 5 | 36,417,176 | C/T | 0.25 | 0.05 | 6.47 | 1.43 | 1.97 × 10⁻⁶ | n/a | 0.91 |
| BICF2G630183623 | 5 | 36,839,546 | T/C | 0.24 | 0.04 | 7.04 | 1.45 | 1.07 × 10⁻⁶ | n/a | Top |
| | | | | | | | | | | SNP |
| BICF2G630183652 | 5 | 36,882,261 | A/G | 0.23 | 0.04 | 6.59 | 1.42 | 4.85 × 10⁻⁶ | n/a | 0.97 |
| BICF2P885817 | 19 | 35,831,635 | T/A | 0.07 | 0.00 | NA* | 2.12 | 7.74 × 10⁻⁶ | n/a | n/a |
| Hemangiosarcoma Analysis. Significantly associated SNPs (p-value <1.0 × 10⁻⁴) | | | | | | | | | | |
| BICF2G63035476 | 5 | 32,708,612 | G/A | 0.31 | 0.50 | 0.46 | 0.84 | 2.03 × 10⁻⁵ | 0.88 | 0.10 |
| BICF2S23317145 | 5 | 32,725,862 | G/A | 0.31 | 0.50 | 0.45 | 0.84 | 1.91 × 10⁻⁵ | 0.88 | 0.10 |
| BICF2P1405079 | 5 | 32,757,545 | T/G | 0.31 | 0.48 | 0.48 | 0.84 | 6.05 × 10⁻⁵ | 0.84 | 0.09 |
| BICF2G63035510 | 5 | 32,757,807 | C/A | 0.31 | 0.48 | 0.48 | 0.84 | 6.05 × 10⁻⁵ | 0.84 | 0.09 |
| BICF2G63035542 | 5 | 32,771,537 | C/T | 0.31 | 0.48 | 0.48 | 0.84 | 6.05 × 10⁻⁵ | 0.83 | 0.09 |
| BICF2G63035564 | 5 | 32,787,898 | A/G | 0.31 | 0.49 | 0.47 | 0.84 | 2.62 × 10⁻⁵ | 0.85 | 0.09 |
| BICF2G63035577 | 5 | 32,804,686 | C/T | 0.31 | 0.49 | 0.47 | 0.84 | 2.62 × 10⁻⁵ | 0.85 | 0.09 |
| BICF2G63035700 | 5 | 32,876,294 | G/T | 0.28 | 0.48 | 0.42 | 0.83 | 4.86 × 10⁻⁶ | 0.98 | 0.09 |
| BICF2G63035705 | 5 | 32,879,166 | G/T | 0.27 | 0.48 | 0.41 | 0.82 | 3.69 × 10⁻⁶ | 0.99 | 0.09 |
| BICF2G63035726 | 5 | 32,901,346 | C/T | 0.27 | 0.48 | 0.40 | 0.82 | 1.40 × 10⁻⁶ | Top SNP | 0.09 |
| BICF2G63035729 | 5 | 32,902,463 | C/T | 0.27 | 0.48 | 0.41 | 0.82 | 3.69 × 10⁻⁶ | 0.99 | 0.09 |
| BICF2G630183626 | 5 | 36,845,402 | C/T | 0.21 | 0.09 | 2.61 | 1.29 | 9.52 × 10⁻⁶ | 0.09 | 0.99 |
| BICF2G630183630 | 5 | 36,848,237 | C/T | 0.21 | 0.09 | 2.75 | 1.31 | 2.11 × 10⁻⁶ | 0.09 | Top |
| | | | | | | | | | | SNP |
| BICF2G630183805 | 5 | 37,081,986 | A/G | 0.20 | 0.10 | 2.32 | 1.26 | 8.55 × 10⁻⁵ | 0.08 | 0.94 |
| BICF2P267306 | 5 | 37,099,612 | A/G | 0.22 | 0.10 | 2.44 | 1.28 | 1.25 × 10⁻⁵ | 0.08 | 0.86 |
| BICF2P1337948 | 5 | 37,111,219 | G/T | 0.23 | 0.10 | 2.57 | 1.30 | 2.59 × 10⁻⁶ | 0.08 | 0.87 |
| BICF2P125723 | 5 | 56,109,903 | T/G | 0.06 | 0.01 | 7.92 | 1.56 | 9.80 × 10⁻⁵ | n/a | n/a |
| BICF2S23516471 | 11 | 36,928,683 | T/C | 0.14 | 0.05 | 3.37 | 1.31 | 9.74 × 10⁻⁵ | n/a | n/a |
| BICF2P22260 | 11 | 40,632,694 | A/T | 0.28 | 0.15 | 2.15 | 1.22 | 3.98 × 10⁻⁵ | n/a | n/a |
| BICF2P858820 | 11 | 40,794,422 | T/C | 0.19 | 0.07 | 3.09 | 1.28 | 2.18 × 10⁻⁵ | n/a | n/a |
| BICF2P260258 | 11 | 41,830,089 | T/C | 0.44 | 0.53 | 0.70 | 0.83 | 4.04 × 10⁻⁵ | n/a | n/a |
| BICF2P1020079 | 11 | 41,864,823 | C/T | 0.52 | 0.41 | 1.56 | 1.20 | 1.82 × 10⁻⁵ | n/a | n/a |
| BICF2P1362415 | 13 | 64,457,753 | T/C | 0.13 | 0.03 | 4.18 | 1.35 | 3.01 × 10⁻⁵ | n/a | n/a |
| BICF2G630746301 | 13 | 64,471,848 | C/T | 0.12 | 0.03 | 4.66 | 1.35 | 4.67 × 10⁻⁵ | n/a | n/a |
| BICF2P354069 | 18 | 52,718,637 | T/C | 0.44 | 0.29 | 1.93 | 1.18 | 8.87 × 10⁻⁵ | n/a | n/a |
| BICF2G630105651 | 25 | 47,612,990 | T/C | 0.40 | 0.57 | 0.50 | 0.86 | 3.19 × 10⁻⁵ | n/a | n/a |
| BICF2S23634252 | 33 | 25,953,846 | A/C | 0.20 | 0.12 | 1.90 | 1.24 | 9.90 × 10⁻⁵ | n/a | n/a |
| Combined Analysis.. Significantly associated SNPs (p-value <1.0 × 10⁻⁴) | | | | | | | | | | |
| BICF2S23035109 | 5 | 11,757,453 | G/A | 0.30 | 0.17 | 2.14 | 1.19 | 7.07 × 10⁻⁵ | n/a | n/a |
| BICF2G63035383 | 5 | 32,622,509 | T/A | 0.32 | 0.49 | 0.49 | 0.85 | 2.23 × 10⁻⁵ | 0.74 | 0.09 |
| BICF2G63035403 | 5 | 32,632,285 | T/C | 0.32 | 0.49 | 0.49 | 0.85 | 2.23 × 10⁻⁵ | 0.74 | 0.09 |
| BICF2G63035476 | 5 | 32,708,612 | G/A | 0.32 | 0.50 | 0.46 | 0.84 | 4.61 × 10⁻⁶ | 0.87 | 0.11 |
| BICF2S23317145 | 5 | 32,725,862 | G/A | 0.32 | 0.50 | 0.46 | 0.84 | 4.33 × 10⁻⁶ | 0.87 | 0.11 |
| BICF2P1405079 | 5 | 32,757,545 | T/G | 0.30 | 0.48 | 0.47 | 0.84 | 7.91 × 10⁻⁶ | 0.82 | 0.10 |
| BICF2G63035510 | 5 | 32,757,807 | C/A | 0.30 | 0.48 | 0.47 | 0.84 | 7.91 × 10⁻⁶ | 0.82 | 0.10 |
| BICF2G63035542 | 5 | 32,771,537 | C/T | 0.30 | 0.48 | 0.47 | 0.84 | 7.91 × 10⁻⁶ | 0.82 | 0.10 |
| BICF2G63035564 | 5 | 32,787,898 | A/G | 0.30 | 0.49 | 0.46 | 0.83 | 2.83 × 10⁻⁶ | 0.83 | 0.10 |
| BICF2G63035577 | 5 | 32,804,686 | C/T | 0.30 | 0.49 | 0.46 | 0.83 | 2.83 × 10⁻⁶ | 0.83 | 0.10 |
| BICF2G63035700 | 5 | 32,876,294 | G/T | 0.28 | 0.48 | 0.43 | 0.82 | 6.89 × 10⁻⁷ | 0.97 | 0.10 |
| BICF2G63035705 | 5 | 32,879,166 | G/T | 0.28 | 0.48 | 0.42 | 0.82 | 5.77 × 10⁻⁷ | 0.98 | 0.10 |
| BICF2G63035726 | 5 | 32,901,346 | C/T | 0.29 | 0.49 | 0.42 | 0.82 | 3.52 × 10⁻⁷ | Top SNP | 0.10 |
| BICF2G63035729 | 5 | 32,902,463 | C/T | 0.29 | 0.48 | 0.43 | 0.82 | 1.10 × 10⁻⁶ | 0.99 | 0.10 |
| BICF2P93507 | 5 | 33,009,401 | A/G | 0.49 | 0.34 | 1.89 | 1.18 | 7.98 × 10⁻⁵ | 0.44 | 0.00 |
| BICF2G630183626 | 5 | 36,845,402 | C/T | 0.22 | 0.09 | 2.95 | 1.29 | 1.43 × 10⁻⁶ | 0.10 | 0.99 |
| BICF2G630183630 | 5 | 36,848,237 | C/T | 0.23 | 0.09 | 3.07 | 1.30 | 4.20 × 10⁻⁷ | 0.10 | Top |
| | | | | | | | | | | SNP |
| BICF2G630183805 | 5 | 37,081,986 | A/G | 0.22 | 0.10 | 2.64 | 1.25 | 1.49 × 10⁻⁵ | 0.09 | 0.95 |
| BICF2P267306 | 5 | 37,099,612 | A/G | 0.24 | 0.10 | 2.72 | 1.27 | 4.33 × 10⁻⁶ | 0.09 | 0.88 |
| BICF2P1337948 | 5 | 37,111,219 | G/T | 0.25 | 0.10 | 2.87 | 1.29 | 7.29 × 10⁻⁷ | 0.09 | 0.88 |
| BICF2P858820 | 11 | 40,794,422 | T/C | 0.18 | 0.07 | 2.90 | 1.25 | 7.57 × 10⁻⁵ | n/a | n/a |

*Due to the absence of MAF in controls

Because both B-cell lymphoma and hemangiosarcoma showed association to the same region of chromosome 5, the datasets for the two diseases were combined. After quality control, the combined dataset included 187 cases (144 hemangiosarcoma cases and 43 B-cell lymphoma cases) and 186 controls, and 127,188 SNPs for the association analysis. The QQ plot deviated from the null distribution at 1×10⁻⁴, identifying 21 SNPs with p-values ranging from 3.5×10⁻⁷ to 8.0×10⁻⁵ to be significant (FIG. 2C, Table 9). Of these 21 SNPs, 19 were located on chromosome 5 between 32.6 Mb and 37.1 Mb. Sixteen SNPs were identical to the significantly associated SNPs from the hemangiosarcoma analysis, but with more significant p-values, confirming their importance also in B-cell lymphoma. The associated SNPs in this region clustered in two peaks located 4 Mb apart. The top two SNPs were located at 32,901,346 bp and 36,848,237 bp, with p-value of 3.5×10⁻⁷ and 4.2×10⁻⁷, respectively.

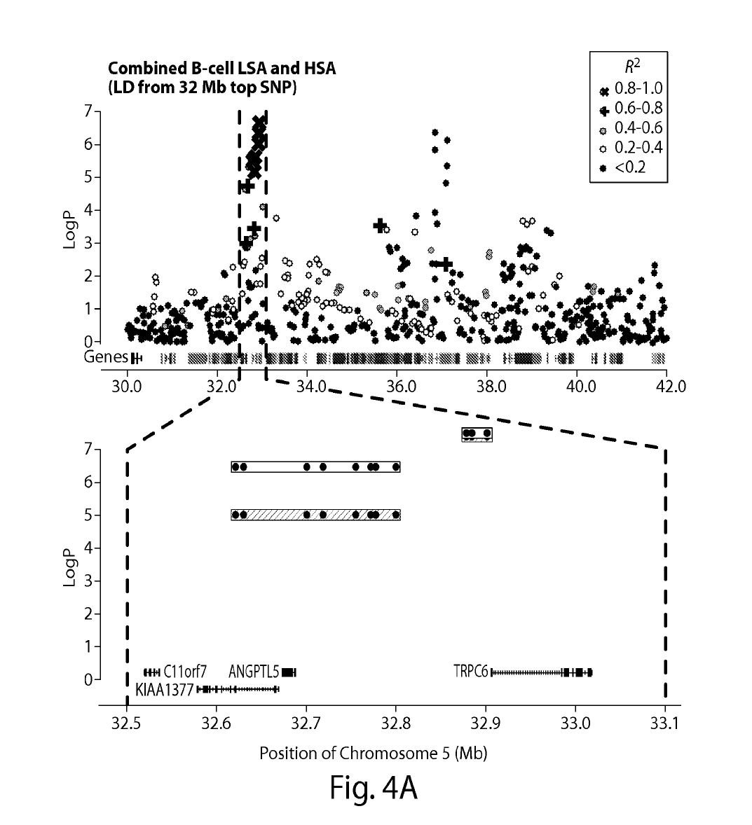

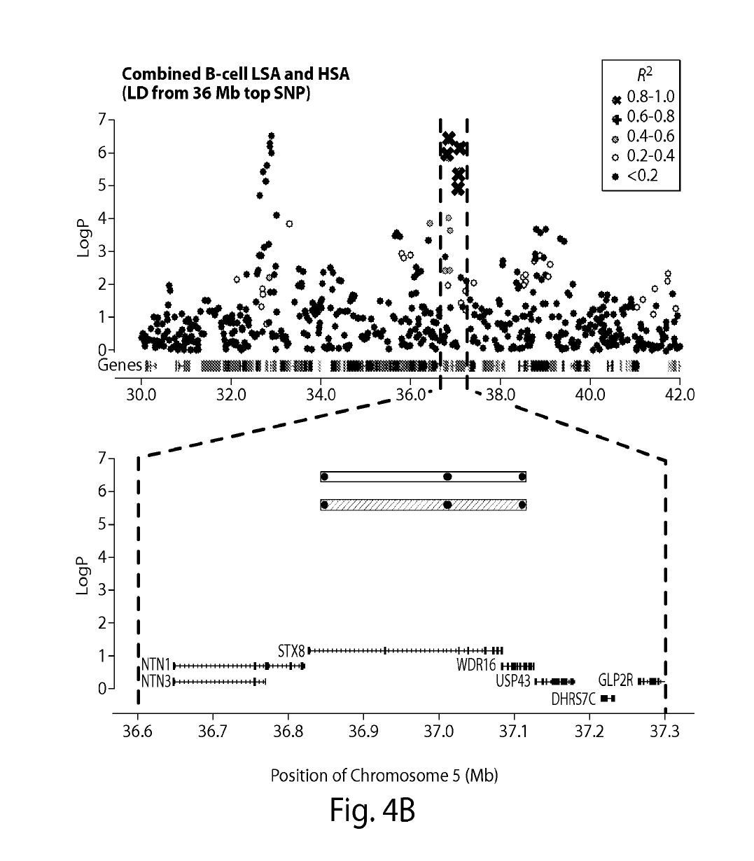

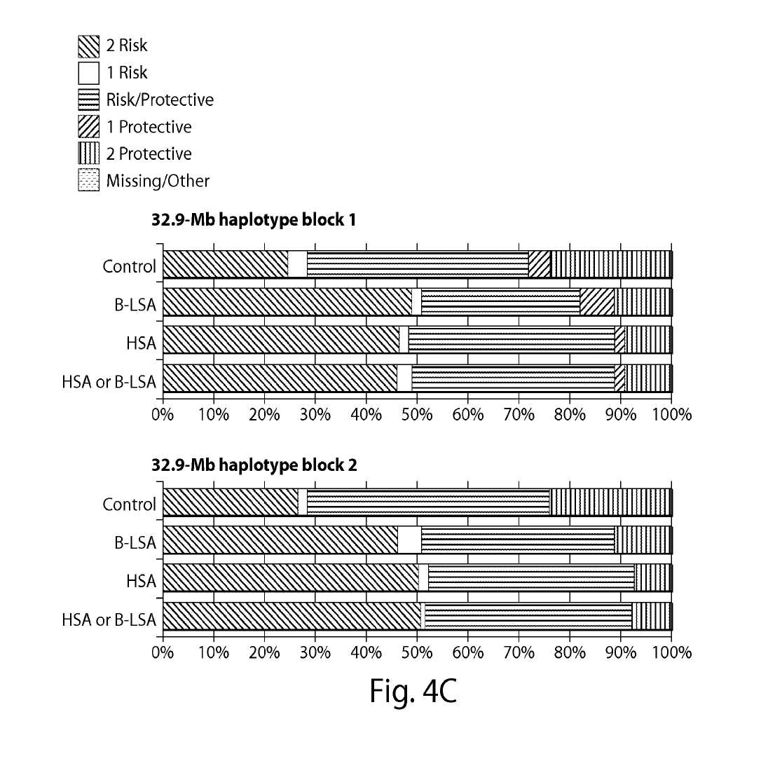

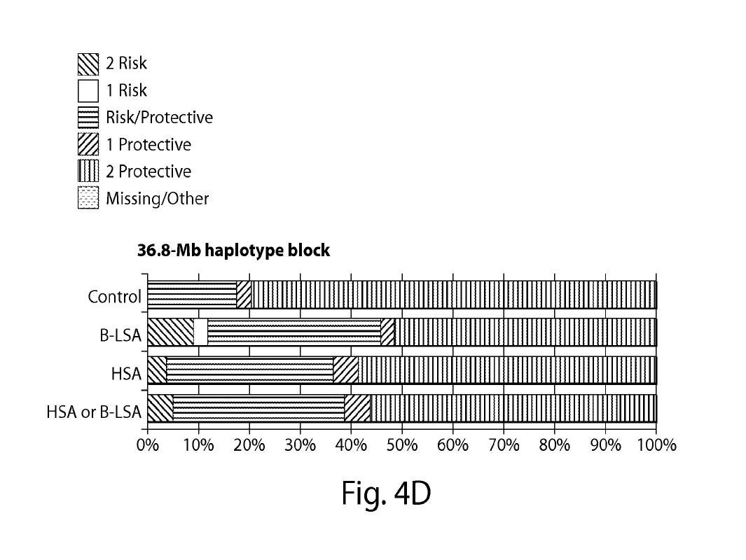

Importantly, the two loci located 4 Mb apart constitute two independent association signals, rather than a single signal due to the long linkage disequilibrium (LD) in dogs. The top SNP in each region show high LD (r2>0.8) with SNPs in the same peak, but low LD (r2<0.2) to the associated SNPs in the other peak (FIG. 4). Association analyses conditioned on the genotype of the top SNP of each peak also indicated independent signals.

To define the exact risk haplotypes and their boundaries, an r2-based clumping analysis was performed by PLINK and Haploview (Purcell; Barrett, Fry et al. 2005; Purcell, Neale et al. 2007) methods) identifying risk and protective haplotypes in both loci. In the 32.9 Mb region two associated haplotype blocks were seen; a 19-SNP block (“32.9 Mb block1”) spanning 182 Kb, and a 4-SNP block (“32.9 Mb block2”) spanning 26 Kb (Table 10 and 11). In the 36.8 Mb region, a 12-SNP haplotype block (“36.8 Mb block1”) spanning 266 Kb was identified (Table 10 and 11).

TABLE 10
List of significantly associated haplotypes

| | | | | | | | p-value |
|---|---|---|---|---|---|---|---|
| | Risk/ | | Frequency | OR | | | (10⁷ |
| | Protective | Frequency | (case, control) | (allelic) | ChiSq | p-value | permutations) |
| Combined | | | | | | | |
| 32.9 Mb | | | | | | | |
| block1 | | | | | | | |
| ACAAGATGT | Risk | 0.57 | 0.67, 0.48 | 2.21 | 27.68 | 1.43 × 10⁻⁷ | 4.00 × 10⁻⁷ |
| TTGGTCCAC | Protective | 0.39 | 0.31, 0.48 | 0.48 | 22.73 | 1.87 × 10⁻⁶ | 8.80 × 10⁻⁶ |
| 32.9 Mb | | | | | | | |
| block2 | | | | | | | |
| TTTT | Risk | 0.61 | 0.71, 0.51 | 2.34 | 30.74 | 2.95 × 10⁻⁸ | <1.00 × 10⁻⁷* |
| GGCC | Protective | 0.38 | 0.28, 0.48 | 0.42 | 31.78 | 1.73 × 10⁻⁸ | <1.00 × 10⁻⁷* |
| 36.8 Mb | | | | | | | |
| block1 | | | | | | | |
| CCAAG | Risk | 0.16 | 0.22, 0.089 | 2.89 | 24.70 | 6.71 × 10⁻⁷ | 2.40 × 10⁻⁶ |
| TTGGT | Protective | 0.83 | 0.76, 0.90 | 0.35 | 26.34 | 2.86 × 10⁻⁷ | 4.00 × 10⁻⁷ |
| | | | | | | | p-value |
| Hemangio- | | | | | | | |
| sarcoma | | | | | | | |
| 32.9 Mb | | | | | | | |
| block1 | | | | | | | |
| ACAAGATGT | Risk | 0.56 | 0.67, 0.48 | 2.25 | 24.96 | 5.86 × 10⁻⁷ | 1.10 × 10⁻⁶ |
| TTGGTCCAC | Protective | 0.40 | 0.31, 0.48 | 0.48 | 19.58 | 9.66 × 10⁻⁶ | 2.98 × 10⁻⁵ |
| 32.9 Mb | | | | | | | |
| block2 | | | | | | | |
| TTTT | Risk | 0.61 | 0.72, 0.51 | 2.47 | 29.60 | 5.32 × 10⁻⁸ | 2.00 × 10⁻⁷ |
| GGCC | Protective | 0.39 | 0.27, 0.48 | 0.41 | 29.18 | 6.60 × 10⁻⁸ | 2.00 × 10⁻⁷ |
| 36.8 Mb | | | | | | | |
| block1 | | | | | | | |
| CCAAG | Risk | 0.14 | 0.20, 0.089 | 2.61 | 17.66 | 2.64 × 10⁻⁵ | 7.37 × 10⁻⁵ |
| TTGGT | Protective | 0.84 | 0.77, 0.90 | 0.39 | 19.17 | 1.19 × 10⁻⁵ | 3.40 × 10⁻⁵ |
| | | | | | | | p-value |
| B-cell | | | | | | | |
| lymphoma | | | | | | | |
| 32.9 Mb | | | | | | | |
| block1 | | | | | | | |
| ACAAGATGT | Risk | 0.51 | 0.65, 0.48 | 2.04 | 8.34 | 0.0039 | 0.077^{#} |
| TTGGTCCAC | Protective | 0.44 | 0.30, 0.48 | 0.48 | 8.52 | 0.0035 | 0.076^{#} |
| 32.9 Mb | | | | | | | |
| block2 | | | | | | | |
| TTTT | Risk | 0.54 | 0.67, 0.51 | 1.96 | 7.30 | 0.0069 | 0.089^{#} |
| GGCC | Protective | 0.45 | 0.30, 0.48 | 0.47 | 9.04 | 0.0026 | 0.021 |
| 36.8 Mb | | | | | | | |
| block1 | | | | | | | |
| CCAAG | Risk | 0.12 | 0.28, 0.089 | 3.96 | 23.23 | 1.44 × 10⁻⁶ | 2.00 × 10⁻⁴ |
| TTGGT | Protective | 0.86 | 0.70, 0.90 | 0.26 | 23.28 | 1.40 × 10⁻⁶ | 2.00 × 10⁻⁴ |

*Permutation test with 10⁷ iterations did not produce any ChiSq value over what is observed.
^{#}For the B-cell lymphoma analysis, haplotypes that are not significantly associated in this analysis are also listed for comparison purposes.

TABLE 11
Haplotype annotated as risk and protective, and their association analysis

| | | Frequency | | | |
|---|---|---|---|---|---|
| | | (case, | OR | OR | |
| Combined | Frequency | control) | (allelic) | (empirical) | p-value |
| 32.9 Mb | | | | | |
| block1 | | | | | |
| PP* | 0.17 | 0.10, 0.24 | 0.34 | 0.81 | 0.39 |
| RP* | 0.41 | 0.39, 0.44 | 0.81 | 1.21 | 0.06 |
| RR* | 0.35 | 0.47, 0.24 | 2.73 | 1.33 | 4.65 × 10⁻⁶ |
| 32.9 Mb | | | | | |
| block2 | | | | | |
| PP | 0.16 | 0.08, 0.24 | 0.27 | 0.47 | 0.12 |
| RP | 0.44 | 0.40, 0.48 | 0.73 | 1.06 | 0.73 |
| RR | 0.39 | 0.50, 0.27 | 2.75 | 1.26 | 0.013 |
| 36.8 Mb | | | | | |
| block1 | | | | | |
| PP | 0.68 | 0.56, 0.80 | 0.33 | 0.60 | 0.07 |
| RP | 0.26 | 0.34, 0.18 | 2.36 | 1.17 | 0.18 |
| RR | 0.02 | 0.05, 0* | n/a* | 57.89* | 0.98* |

*PP: homozygous protective, RR: homozygous risk, RP: heterogygous (see Table 10)

The risk haplotype at the 32.9 Mb locus had high frequency (FIGS. 4C and 4D). The frequency was 65.1% in the 43 dogs with B-cell lymphoma (49% homozygous, 33% heterozygous for the risk allele) and 67.4% in the 144 dogs with hemangiosarcoma (46% homozygous, 43% heterozygous) as compared to 50.3% in the 186 control dogs (26% homozygous, 48% heterozygous) for block 1. For block 2, the frequencies were similar with 67.4% for B-cell lymphoma (46% homozygous, 42% heterozygous), 72.2% for hemangiosarcoma (47% homozygous, 42% heterozygous) and 51.3% in control dogs (27% homozygous, 49% heterozygous)

In contrast, the risk haplotype at the 36.8 Mb locus had a much lower frequency: 27.9% in dogs with B-cell lymphoma (9% homozygous, 37% are heterozygous) and 20.1% in dogs with hemangiosarcoma (3% homozygous, 33% heterozygous) as compared to 8.9% in controls (0% homozygous, 18% are heterozygous) (FIGS. 4C and 4D). The disparate frequency of the risk alleles at the two loci also supported a hypothesis of two distinct risk factors.

To determine the proportion of disease risk explained by the genotypes of these two loci, a restricted maximum likelihood (REML) analysis was performed using GCTA software (Yang, Lee et al. 2011). Together, the two loci accounted for 52.5%±17.8% of the risk for canine B-cell lymphoma and hemangiosarcoma, suggesting that these risk loci are major drivers of disease in the golden retriever breed. The risk contributed by these two loci may be slightly higher for hemangiosarcoma (56%±20%) than for B-cell lymphoma (46%±32%).

### Germ-Line Risk Factors Influence Expressions of Genes Located Both Cis and Trans

To evaluate potential candidate genes within the regions of association, two approaches were taken. First the coding exons of genes within the most associated regions were examined for risk-haplotype-concordant amino acid changing germ-line mutations using ˜40× coverage of Illumina sequence. None of the genes near the 36.8 Mb locus (NTN1, NTN3, STX8, and WDR16) had any amino acid changes. At the 32.9 Mb locus, the three genes, KIAA1377, ANGPTL5 and TRPC6 each had one SNP leading to amino acid substitutions. However, none of these variants were associated with the risk haplotype.

Because no coding changes were evident, it was investigated whether the risk haplotypes were associated with transcriptional changes in tumors. 22 B-cell lymphoma samples were studied, from which RNA-Seq and correlated expression levels for protein-coding genes genome-wide with the risk haplotypes were obtained. For the 32.9 Mb locus, 12 samples that were homozygous for the risk allele were compared to the remaining 10 samples (8 heterozygous, 2 lacking the risk allele). The risk haplotype (both block 1 and 2) in homozygous state significantly altered the expression of TRPC6, the closest gene to block 2 (log FCrisk=−6.70, p-value=2.85×10-16, FDR=5.38×10-12, Table 4, FIG. 3). In fact, the expression of the TRPC6 transcript was almost null in the tumor in dogs that are homozygous risk. TRPC6 encodes a transient receptor potential channel, which mediates calcium ion (Ca2+) influx to T-cells through a couple of independent pathways; PLCγ pathway regulated by the T-cell receptor, and PI3K pathway downstream of CD28, a T-cell co-stimulatory molecule (Carrillo, Hichami et al. 2012). TRPC6 is also activated through CXC-t e G-protein-coupled, and CC-type chemokine receptors, which are widely expressed by various immune cells including T-cells (Damann, Owsianik et al. 2009; Yao, Peng et al. 2009). The elevation of intracellular Ca2+ concentration is a key and necessary event in T-cell activation, leading to the activation of calcineurin and NFAT (nuclear factor of activated T-cell). The expression levels of TRPC6 have been shown to significantly alter levels of intracellular Ca2+ elevation and T-cell activation (Tseng, Lin et al. 2004; Carrillo, Hichami et al. 2012).

In addition, the expression levels of three nearby genes KIAA1377, ANGPTL5 and BIRC3 were also reduced significantly or near significantly by the risk haplotype, respectively (KIAA1377: log FCrisk=−2.25, p-value=1.98×10-6, FDR=0.004, ANGPTL5: log FCrisk=−2.60, p-value=1.30×10-4, FDR=0.06, and BIRC3: log FCrisk=−0.76, p-value=1.05×10-3, FDR=0.21, Table 4, FIG. 3). ANGPTL5 is a member of the angiopoietin growth factor family (Zeng, Dai et al. 2003). It affects plasma triglyceride levels (Romeo, Yin et al. 2009) and stimulates growth of hematopoietic stem cells in culture (Zhang, Kaba et al. 2008; Drake, Khoury et al. 2011). KIAA1377 is a novel centrosomal protein associating with centromere and kinetochore proteins (Tipton, Wang et al. 2012) and is required for cytokinesis (Chen, Lee et al. 2009). BIRC3 encodes an inhibitor of apoptosis. Thus, the predisposing mutation(s) tagged by the 32.9 Mb block1 and block 2 haplotypes may play a role in B-cell lymphoma by regulating multiple nearby genes.

Across the genome 28 additional genes had significantly altered expression linked to the 32.9 Mb risk haplotypes (Table 12 and 13). All genes, except for PIK3R6, are located on other chromosomes, suggestive of trans-regulation or downstream effects triggered by the predisposing mutation(s). Pathway analysis using Ingenuity Pathway Analysis (IPA; Ingenuity Systems) did not cluster significantly in any particular canonical pathways, nor was any common downstream biological functions identified. Instead nine micro RNAs (miRNAs) were identified as the upstream regulator of the genes with the observed expression changes.

TABLE 12
Top 10 differentially expressed genes by the risk haplotype at each locus

| | | | | | Start of first |
|---|---|---|---|---|---|
| Gene Name | logFC* | p-value | FDR | Chr | exon |
| Analysis by the risk | | | | | |
| status at 32.9 Mb | | | | | |
| TRPC6 | −6.70 | 2.85 × 10⁻¹⁶ | 5.38 × 10⁻¹² | 5 | 32,980,482 |
| C1GALT1 | −6.51 | 4.10 × 10⁻¹⁴ | 3.87 × 10⁻¹⁰ | 14 | 28,774,669 |
| RPL6 | 1.48 | 3.36 × 10⁻⁷ | 2.11 × 10⁻³ | 26 | 12,985,679 |
| PIK3R6 | −1.66 | 4.96 × 10⁻⁷ | 2.30 × 10⁻³ | 5 | 36,465,452 |
| ENSCAFG00000029323 | 6.04 | 6.10 × 10⁻⁷ | 2.30 × 10⁻³ | 26 | 28,182,315 |
| XLOC_011971 | 5.12 | 8.91 × 10⁻⁷ | 2.80 × 10⁻³ | 11 | 76,536,413 |
| FGFR4 | −3.82 | 1.21 × 10⁻⁶ | 2.96 × 10⁻³ | 4 | 39,432,031 |
| SCARA5 | −2.65 | 1.25 × 10⁻⁶ | 2.96 × 10⁻³ | 25 | 32,580,352 |
| GFRA2 | −3.31 | 1.63 × 10⁻⁶ | 3.42 × 10⁻³ | 25 | 38,426,197 |
| KIAA1377 | −2.25 | 1.98 × 10⁻⁶ | 3.74 × 10⁻³ | 5 | 32,568,213 |
| Analysis by the risk | | | | | |
| status at 36.8 Mb | | | | | |
| ENSCAFG00000013622 | 5.60 | 7.63 × 10⁻¹² | 1.44 × 10⁻⁷ | 26 | 30,231,942 |
| CD5L | −3.35 | 7.52 × 10⁻⁷ | 4.49 × 10⁻³ | 7 | 43,484,935 |
| XLOC_102336 | 6.32 | 9.14 × 10⁻⁷ | 4.49 × 10⁻³ | X | 53,591,387 |
| CXL10 | −3.33 | 9.51 × 10⁻⁷ | 4.49 × 10⁻³ | 32 | 3,561,942 |
| SLC25A48 | −3.84 | 1.60 × 10⁻⁶ | 5.59 × 10⁻³ | 11 | 26,765,802 |
| KRT24 | −5.01 | 1.79 × 10⁻⁶ | 5.59 × 10⁻³ | 9 | 30,231,942 |
| ENSCAFG00000029323 | 6.02 | 2.07 × 10⁻⁶ | 5.59 × 10⁻³ | 26 | 43,484,935 |
| RP11-10N16.3 | −4.97 | 2.76 × 10⁻⁶ | 6.49 × 10⁻³ | 2 | 53,591,387 |
| HIST1H | 2.74 | 3.09 × 10⁻⁶ | 6.49 × 10⁻³ | 35 | 3,561,942 |
| HS3ST3B1 | 1.96 | 4.15 × 10⁻⁶ | 7.77 × 10⁻³ | 5 | 26,765,802 |

*Fold change was calculated by designating the non-risk group as a reference.

Expression comparison between the 36.8 Mb locus risk (1 homozygous and 5 heterozygous dogs) and non-risk (16 dogs lacking the risk haplotype) haplotypes identified 89 alternatively expressed genes elsewhere in the genome, with no sign of cis-regulation (Table 13). However, the IPA analysis of the 89 genes showed that the expression changes of many of those genes were linked to decrease in the activation of immune cells. The analysis strongly associated various immune modulators to the observed expression changes, including TCR (poverlap=6.24×10-10), cytokines; IL2 (poverlap=1.53×10-9), IL15 (poverlap=9.41×10-6), which is essential for activation and survival of T-cells and NK cells, and TNF (poverlap=5.83×10-5). Seven miRNAs, four of which have been associated with lymphoma/leukemia were also identified to be the regulators. Significant enrichment of the differentially expressed genes was also observed in four canonical pathways that play role in innate and adaptive immunity, and hematopoiesis. All of these changes are consistent with the role of STX8 in fusing lytic granules with the plasma membrane in CD8 T-cells for cytotoxic release.

TABLE 13
Differentially expressed genes by the risk haplotype at each locus

| | | | | | Start |
|---|---|---|---|---|---|
| Gene Name | logFC* | p-value | FDR | Chr | first exon |
| 32.9 Mb risk analysis | | | | | |
| TRPC6 | −6.70 | 2.85 × 10⁻¹⁶ | 5.38 × 10⁻¹² | 5 | 32,980,482 |
| C1GALT1 | −6.51 | 4.10 × 10⁻¹⁴ | 3.87 × 10⁻¹⁰ | 14 | 28,774,669 |
| RPL6 | 1.48 | 3.36 × 10⁻⁷ | 2.11 × 10⁻³ | 26 | 12,985,679 |
| PIK3R6 | −1.66 | 4.96 × 10⁻⁷ | 2.30 × 10⁻³ | 5 | 36,465,452 |
| ENSCAFG00000029323 | 6.04 | 6.10 × 10⁻⁷ | 2.30 × 10⁻³ | 26 | 28,182,315 |
| XLOC_011971 | 5.12 | 8.91 × 10⁻⁷ | 2.80 × 10⁻³ | 11 | 76,536,413 |
| FGFR4 | −3.82 | 1.21 × 10⁻⁶ | 2.96 × 10⁻³ | 4 | 39,432,031 |
| SCARA5 | −2.65 | 1.25 × 10⁻⁶ | 2.96 × 10⁻³ | 25 | 32,580,352 |
| GFRA2 | −3.31 | 1.63 × 10⁻⁶ | 3.42 × 10⁻³ | 25 | 38,426,197 |
| KIAA1377 | −2.25 | 1.98 × 10⁻⁶ | 3.74 × 10⁻³ | 5 | 32,568,213 |
| GRM5 | −2.66 | 2.85 × 10⁻⁶ | 4.89 × 10⁻³ | 21 | 13,977,304 |
| GPC3 | −3.69 | 6.34 × 10⁻⁶ | 9.40 × 10⁻³ | X | 107,374,933 |
| ENSCAFG00000030890 | −6.35 | 6.47 × 10⁻⁶ | 9.40 × 10⁻³ | unknown | |
| FABP4 | −3.25 | 8.38 × 10⁻⁶ | 1.09 × 10−2 | 29 | 31,653,357 |
| HTR4 | −2.73 | 8.62 × 10⁻⁶ | 1.09 × 10⁻² | 4 | 63,478,926 |
| U2 | 4.88 | 9.52 × 10⁻⁶ | 1.12 × 10⁻² | 9 | 23,182,999 |
| CD300A | 1.59 | 1.41 × 10⁻⁵ | 1.57 × 10⁻² | 9 | 8,905,899 |
| Q95J95 | −4.09 | 1.51 × 10⁻⁵ | 1.58 × 10⁻² | 34 | 22,399,471 |
| ZNF662 | −2.22 | 1.67 × 10⁻⁵ | 1.66 × 10⁻² | 23 | 15,012,783 |
| XLOC_026187 | −1.72 | 1.96 × 10⁻⁵ | 1.85 × 10⁻² | 17 | 66,746,909 |
| MPO | 3.26 | 2.14 × 10⁻⁵ | 1.93 × 10⁻² | 9 | 36,248,220 |
| KIF5C | −1.73 | 2.40 × 10⁻⁵ | 2.03 × 10⁻² | 19 | 53,557,570 |
| CACNA1D | −2.26 | 2.48 × 10⁻⁵ | 2.03 × 10⁻² | 20 | 39,185,673 |
| XLOC_044225 | −3.02 | 2.68 × 10⁻⁵ | 2.10 × 10⁻² | 23 | 15,037,566 |
| XLOC_067564 | 3.78 | 2.99 × 10⁻⁵ | 2.26 × 10⁻² | 32 | 6,610,929 |
| NETO1 | 4.42 | 3.38 × 10⁻⁵ | 2.39 × 10⁻² | 1 | 9,117,342 |
| RGS13 | −5.41 | 3.42 × 10⁻⁵ | 2.39 × 10⁻² | 38 | 9,287,851 |
| COL6A6 | −2.18 | 4.74 × 10⁻⁵ | 3.20 × 10⁻² | 23 | 30,810,556 |
| KIAA1456 | −1.76 | 6.40 × 10⁻⁵ | 4.17 × 10⁻² | 16 | 39,369,371 |
| ADAMTS2 | −1.60 | 7.89 × 10⁻⁵ | 4.97 × 10⁻² | 11 | 5,283,211 |
| 36.9 Mb risk analysis | | | | | |
| ENSCAFG00000013622 | 5.60 | 7.63 × 10⁻¹² | 1.44 × 10⁻⁷ | 26 | 30,231,942 |
| CD5L | −3.35 | 7.52 × 10⁻⁷ | 4.49 × 10⁻³ | 7 | 43,484,935 |
| XLOC_102336 | 6.32 | 9.14 × 10⁻⁷ | 4.49 × 10⁻³ | X | 53,591,387 |
| CXL10 | −3.33 | 9.51 × 10⁻⁷ | 4.49 × 10⁻³ | 32 | 3,561,942 |
| SLC25A48 | −3.84 | 1.60 × 10⁻⁶ | 5.59 × 10⁻³ | 11 | 26,765,802 |
| KRT24 | −5.01 | 1.79 × 10⁻⁶ | 5.59 × 10⁻³ | 9 | 30,231,942 |
| ENSCAFG00000029323 | 6.02 | 2.07 × 10⁻⁶ | 5.59 × 10⁻³ | 26 | 43,484,935 |
| RP11-10N16.3 | −4.97 | 2.76 × 10⁻⁶ | 6.49 × 10⁻³ | 2 | 53,591,387 |
| HIST1H | 2.74 | 3.09 × 10⁻⁶ | 6.49 × 10⁻³ | 35 | 3,561,942 |
| HS3ST3B1 | 1.96 | 4.15 × 10⁻⁶ | 7.77 × 10⁻³ | 5 | 26,765,802 |
| CCR6 | 1.96 | 4.15 × 10⁻⁶ | 7.77 × 10⁻³ | 1 | 57,980,038 |
| XLOC_083025 | 1.06 | 4.53 × 10⁻⁶ | 7.77 × 10⁻³ | 5 | 11,840,549 |
| ENSCAFG00000028509 | −3.07 | 4.96 × 10⁻⁶ | 7.81 × 10⁻³ | 8 | 76,462,898 |
| PADI4 | −3.34 | 5.42 × 10⁻⁶ | 7.87 × 10⁻³ | 2 | 83,808,650 |
| XLOC_022131 | 2.13 | 6.82 × 10⁻⁶ | 9.20 × 10⁻³ | 16 | 11,680,172 |
| XLOC_068212 | 2.69 | 7.57 × 10⁻⁶ | 9.28 × 10⁻³ | 33 | 16,327,880 |
| PROK2 | 1.75 | 8.13 × 10⁻⁶ | 9.28 × 10⁻³ | 20 | 23,252,674 |
| XLOC_088759 | −5.80 | 8.35 × 10⁻⁶ | 9.28 × 10⁻³ | 6 | 50,988,431 |
| GZMA | 5.37 | 1.16 × 10⁻⁵ | 1.17 × 10⁻² | 2 | 45,338,897 |
| OBSL1 | −2.65 | 1.17 × 10⁻⁵ | 1.17 × 10⁻² | 37 | 29,048,300 |
| KIAA1598 | −1.23 | 1.46 × 10⁻⁵ | 1.36 × 10⁻² | 28 | 30,422,388 |
| U6 | −3.06 | 1.52 × 10⁻⁵ | 1.36 × 10⁻² | 1 | 108,582,089 |
| NPDC1 | 1.93 | 1.62 × 10⁻⁵ | 1.39 × 10⁻² | 9 | 51,929,374 |
| PGBD5 | −1.06 | 1.70 × 10⁻⁵ | 1.40 × 10⁻² | 4 | 11,919,640 |
| XLOC_094643 | −4.05 | 1.78 × 10⁻⁵ | 1.40 × 10⁻² | 8 | 73,700,104 |
| LBH | 1.84 | 1.91 × 10⁻⁵ | 1.45 × 10⁻² | 17 | 27,046,602 |
| GPR27 | −1.65 | 2.04 × 10⁻⁵ | 1.48 × 10⁻² | 20 | 23,274,848 |
| PTPN22 | −3.15 | 2.15 × 10⁻⁵ | 1.50 × 10⁻² | 17 | 54,698,307 |
| CSF1 | 1.05 | 3.28 × 10⁻⁵ | 2.01 × 10⁻² | 6 | 45,087,921 |
| KLRK1 | −1.21 | 3.37 × 10⁻⁵ | 2.01 × 10⁻² | 27 | 38,653,807 |
| CNNM1 | −2.67 | 3.42 × 10⁻⁵ | 2.01 × 10⁻² | 28 | 15,286,158 |
| B6F250 | −7.13 | 3.43 × 10⁻⁵ | 2.01 × 10⁻² | 11 | 77,297,376 |
| ENSCAFG00000029236 | −1.97 | 3.52 × 10⁻⁵ | 2.01 × 10⁻² | 26 | 29,827,617 |
| CD8A | −4.27 | 3.52 × 10⁻⁵ | 2.01 × 10⁻² | 17 | 41,434,534 |
| ENSCAFG00000031437 | −2.47 | 4.07 × 10⁻⁵ | 2.26 × 10⁻² | 13 | 39,940,336 |
| GALNT13 | −2.30 | 4.72 × 10⁻⁵ | 2.55 × 10⁻² | 36 | 3,612,369 |
| EXTL1 | −8.28 | 5.06 × 10⁻⁵ | 2.61 × 10⁻² | 2 | 76,822,410 |
| RAB19 | −2.63 | 5.23 × 10⁻⁵ | 2.61 × 10⁻² | 16 | 11,508,662 |
| XLOC_100547 | −3.02 | 5.45 × 10⁻⁵ | 2.61 × 10⁻² | unknown | |
| CCL22 | −2.92 | 5.51 × 10⁻⁵ | 2.61 × 10⁻² | 2 | 61,919,540 |
| ENSCAFG00000031494 | −2.57 | 5.67 × 10⁻⁵ | 2.61 × 10⁻² | 35 | 27,145,025 |
| MT1 | 1.13 | 5.67 × 10⁻⁵ | 2.61 × 10⁻² | 2 | 62,483,038 |
| EOMES | −2.23 | 5.88 × 10⁻⁵ | 2.61 × 10⁻² | 23 | 19,517,472 |
| XLOC_091705 | −2.45 | 6.02 × 10⁻⁵ | 2.61 × 10⁻² | 7 | 43,797,173 |
| RP11-664D7.4 | 1.68 | 6.09 × 10⁻⁵ | 2.61 × 10⁻² | 29 | 20,809,866 |
| TNFAIP3 | −4.57 | 6.65 × 10⁻⁵ | 2.79 × 10⁻² | 1 | 33,290,328 |
| FAM190A | −1.10 | 7.53 × 10⁻⁵ | 3.09 × 10⁻² | 32 | 16,312,691 |
| XLOC_077615 | −1.92 | 7.89 × 10⁻⁵ | 3.17 × 10⁻² | 4 | 56,275,195 |
| ENSCAFG00000029651 | 1.43 | 8.22 × 10⁻⁵ | 3.23 × 10⁻² | 16 | 9,847,648 |
| GZMK | −2.60 | 8.67 × 10⁻⁵ | 3.34 × 10⁻² | 4 | 63,762,701 |
| GZMB | −2.47 | 9.03 × 10⁻⁵ | 3.41 × 10⁻² | 8 | 7,514,312 |
| CCDC168 | −2.90 | 9.26 × 10⁻⁵ | 3.43 × 10⁻² | 22 | 55,175,232 |
| MARCKSL1 | 1.72 | 9.80 × 10⁻⁵ | 3.56 × 10⁻² | 2 | 71,790,975 |
| MAPK11 | −1.49 | 1.11 × 10⁻⁴ | 3.86 × 10⁻² | 10 | 19,967,115 |
| TRBC2 | −2.51 | 1.12 × 10⁻⁴ | 3.86 × 10⁻² | 16 | 9,743,931 |
| SCN2A | −1.93 | 1.14 × 10⁻⁴ | 3.86 × 10⁻² | 36 | 13,482,292 |
| CD151 | 3.63 | 1.16 × 10⁻⁴ | 3.86 × 10⁻² | 18 | 48,235,379 |
| TBXA2R | −0.91 | 1.18 × 10⁻⁴ | 3.86 × 10⁻² | 20 | 58,884,471 |
| TNFRSF21 | −2.51 | 1.19 × 10⁻⁴ | 3.86 × 10⁻² | 12 | 18,346,948 |
| ENSCAFG00000029467 | −1.10 | 1.21 × 10⁻⁴ | 3.86 × 10⁻² | 26 | 29,217,092 |
| NKG7 | −3.63 | 1.24 × 10⁻⁴ | 3.89 × 10⁻² | 1 | 108,487,191 |
| CHGA | −2.38 | 1.37 × 10⁻⁴ | 4.13 × 10⁻² | 8 | 4,960,139 |
| CCL5 | −1.59 | 1.39 × 10⁻⁴ | 4.13 × 10⁻² | 9 | 41,137,795 |
| H6BA90 | −2.36 | 1.43 × 10⁻⁴ | 4.13 × 10⁻² | 3 | 76,386,132 |
| PLEKHG5 | 0.87 | 1.44 × 10⁻⁴ | 4.13 × 10⁻² | 5 | 63,315,232 |
| SMOC1 | −1.48 | 1.45 × 10⁻⁴ | 4.13 × 10⁻² | 8 | 46,642,731 |
| TNIK | −2.86 | 1.49 × 10⁻⁴ | 4.13 × 10⁻² | 34 | 38,431,778 |
| CCL19 | −1.75 | 1.50 × 10⁻⁴ | 4.13 × 10⁻² | 11 | 54,369,910 |
| ENSCAFG00000028940 | −1.65 | 1.51 × 10⁻⁴ | 4.13 × 10⁻² | 1 | 106,256,696 |
| XLOC_024761 | −2.24 | 1.54 × 10⁻⁴ | 4.13 × 10⁻² | 16 | 61,735,846 |
| RGS10 | −2.75 | 1.56 × 10⁻⁴ | 4.13 × 10⁻² | 28 | 32,664,957 |
| TMPRSS13 | −1.58 | 1.58 × 10⁻⁴ | 4.13 × 10⁻² | 5 | 18,732,119 |
| DLGAP3 | 3.11 | 1.59 × 10⁻⁴ | 4.13 × 10⁻² | 15 | 10,066,983 |
| ENSCAFG00000028850 | 2.54 | 1.61 × 10⁻⁴ | 4.13 × 10⁻² | 26 | 30,457,438 |
| ENSCAFG00000030894 | −2.81 | 1.63 × 10⁻⁴ | 4.13 × 10⁻² | 8 | 76,822,609 |
| SLC38A11 | −3.29 | 1.64 × 10⁻⁴ | 4.13 × 10⁻² | 36 | 13,055,671 |
| KEL | 1.67 | 1.74 × 10⁻⁴ | 4.26 × 10⁻² | 16 | 9,604,993 |
| ABCA4 | −1.41 | 1.75 × 10⁻⁴ | 4.26 × 10⁻² | 6 | 58,112,925 |
| TNFRSF18 | −2.23 | 1.76 × 10⁻⁴ | 4.26 × 10⁻² | 5 | 59,395,487 |
| TNFRSF4 | −1.72 | 1.80 × 10⁻⁴ | 4.26 × 10⁻² | 5 | 59,402,594 |
| AFF2 | −2.22 | 1.85 × 10⁻⁴ | 4.26 × 10⁻² | X | 119,767,004 |
| CXCR3 | −3.37 | 1.87 × 10⁻⁴ | 4.26 × 10⁻² | X | 58,807,999 |
| TCTEX1D4 | 1.72 | 1.87 × 10⁻⁴ | 4.26 × 10⁻² | 15 | 18,522,379 |
| FBXO11 | −2.67 | 1.87 × 10⁻⁴ | 4.26 × 10⁻² | 10 | 53,052,977 |
| CHRM4 | 1.48 | 1.90 × 10⁻⁴ | 4.27 × 10⁻² | 18 | 46,110,510 |
| CD8B | −2.26 | 1.96 × 10⁻⁴ | 4.36 × 10⁻² | 17 | 41,407,816 |
| HTRA1 | −2.08 | 2.11 × 10⁻⁴ | 4.59 × 10⁻² | 28 | 35,122,204 |
| LAT | −1.18 | 2.12 × 10⁻⁴ | 4.59 × 10⁻² | 6 | 21,470,908 |
| LAD1 | −1.99 | 2.31 × 10⁻⁴ | 4.95 × 10⁻² | 7 | 4,486,977 |

*Fold change was calculated by designating the non-risk group as a reference.

Taken together the almost complete expression decrease of TRPC6 by the homozygous risk state at 32.9 Mb locus, and the expression changes correlated with at least one risk allele at the 36.8 Mb locus strongly suggest that the T-cell activation is severely compromised in the tumors of golden retrievers with B-cell lymphoma.

### Discussion

GWAS of human DLBCL in thousands of human patients have detected tens of loci which together account for <10% of the genetic risk. For human angiosarcoma no GWAS has been performed due to the rarity of the disease. As described herein, GWAS was performed for canine lymphoma and hemangiosarcoma using less than 400 dogs for both diseases combined, and two loci of strong effect accounting for approximately half of the disease risk were identified. The fact that one of the two risk factors on chromosome 5 (32 Mb) is very common in the U.S. golden retriever population may relate to the use of popular sires and may constitute an example of an allele accumulating either through drift or selective breeding for a nearby locus.

Incorporation of additional cases and controls in the future will likely identify additional risk factors, as several candidate loci fall just above or below the significance threshold. In this context it is noted that the 43 B-cell lymphoma cases alone produced a relatively weaker signal for the canine chromosome 5 locus at 32 Mb, suggesting that for this high frequency risk allele a higher sample number would be optimal as predicted by original power calculations that 100 cases and 100 controls are required for detection of a risk factor conferring a 5-fold increased risk.

In this study, the individual risk factors appear to contribute a risk of 1.5- to 7-fold depending on if a simple allelic risk or a context-dependent OR was calculated. The remarkable finding lies partly in the fact that only two loci contribute as much as 50% of the total risk.

The fact that both hemangiosarcoma and B-cell lymphoma predisposition map to the same loci is intriguing. Both diseases stem from the hematopoietic lineage, where hemangioblasts are proposed to have the ability to generate both lymphocytic stem cells and endothelial cells. Previous studies have also proposed that canine hemangiosarcoma carry hematopoietic stem cell markers consistent with an immature precursor for these cells.

Interestingly, the 32 Mb region contained two associated haplotypes, which could either constitute the same larger haplotype tagging a single mutation or each haplotype could be carrying different mutations. While in theory if there were two different mutations, these could be affecting the two different diseases differently, the multipotent function of the genes affected by the expression changes linked to the risk haplotype suggests that even a single mutation could easily have pleiotropic effects in different tissues.

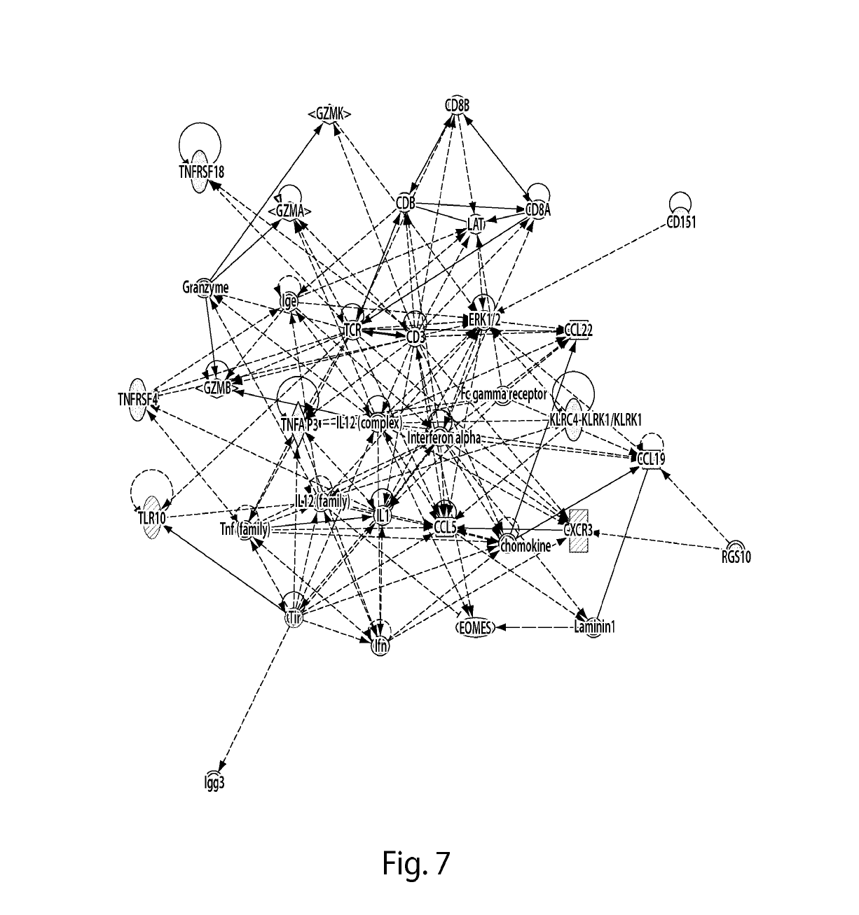

RNA-Seq data from B-cell lymphoma tumors demonstrated an almost complete reduction of TRPC6 transcript associated with the 32 Mb risk haplotype. Intriguingly, TRPC6 is not normally expressed in B-cells (Roedding, Li et al. 2006), but has been reported to play a major role for T-cell and NK-cell activation (Tseng, Lin et al. 2004; Finney-Hayward, Popa et al. 2010; Carrillo, Hichami et al. 2012). Without wishing to be bound by any theory or mechanism, it is hypothesized that B-cell lymphoma is at least partially promoted by a lack of T-cell and/or NK-cell response in the lymph nodes. This hypothesis is further supported by the effect of the 36 Mb locus, where ˜90 genes involved in T-cell activation were suppressed by the risk allele (FIG. 7). Interestingly, no coding mutation or direct expression change was seen in the STX8 gene, which overlaps the risk haplotype, although this gene acts as a SNARE promoting the docking and release of lytic granules at the plasma membrane in CD8+ T-cells.

The 32 Mb risk allele also correlated, although less strongly, with a reduced expression in other genes near the 32 Mb locus in B-cell lymphoma tumors. These genes include genes involved in both in B-cell anti-apoptotic signaling in human DLBCL (BIRC3) a potent hematopoietic stem cells growth factor (ANGPTL5) (Zhang, Kaba et al. 2008; Drake, Khoury et al. 2011; Khoury, Drake et al. 2011), a novel centrosomal protein with mitotic regulatory potential (KIAA1377) (Tipton, Wang et al. 2012) and could affect other aspects of tumor formation. The risk haplotype contained a potential lincRNA not previously reported, which hypothetically could be involved in regulating the expression of multiple genes in the region.

### Methods Summary

All golden retrievers in this study were privately owned pet dogs in the U.S., and participated in this study with owner consent. The diagnosis of B-cell lymphoma or hemangiosarcoma was confirmed by histology including immunohistochemistry and PCR based methods (Supplemental methods). Genomic DNA samples were isolated from whole blood and genotyped for 170,000 SNPs using the Illumina 170K canine HD array (Vaysse, Ratnakumar et al. 2011). To successfully control for the population stratification present in the dataset, analysis approach was taken based on a method described by Price et al. (Price, Zaitlen et al. 2010). The discovery of germ-line was performed by generating 40× whole genome sequencing by Illumina HiSeq of DNA samples from three dogs with that had B-cell lymphoma and had been included in the GWAS. The gene expression levels of twenty-two B-cell lymphoma tumors were profiled by strand-specific RNA-Seq, and analyzed for changes by the germ-line risk alleles at 32.9 Mb and 36.8 Mb loci on chromosome 5. To test if the genes affected by the germ-line risk alleles were enriched in particular biological pathways/biological functions, Ingenuity Pathway Analysis (IPA), proteins encoded in genomic regions associated with immune-mediated were used.

### REFERENCES

Conde L, Halperin E, Akers N K, Brown K M, Smedby K E, Rothman N, Nieters A, Slager S L, Brooks-Wilson A, Agana L, Riby J, Liu J, Adami H O, Darabi H, Hjalgrim H, Low H Q, Humphreys K, Melbye M, Chang E T, Glimelius B, Cozen W, Davis S, Hartge P, Morton L M, Schenk M, Wang S S, Armstrong B, Kricker A, Milliken S, Purdue M P, Vajdic C M, Boyle P, Lan Q, Zahm S H, Zhang Y, Zheng T, Becker N, Benavente Y, Boffetta P, Brennan P, Butterbach K, Cocco P, Foretova L, Maynadié M, de Sanjosë S, Staines A, Spinelli J J, Achenbach S J, Call T G, Camp N J, Glenn M, Caporaso N E, Cerhan J R, Cunningham J M, Goldin L R, Hanson C A, Kay N E, Lanasa M C, Leis J F, Marti G E, Rabe K G, Rassenti L Z, Spector L G, Strom S S, Vachon C M, Weinberg J B, Holly E A, Chanock S, Smith M T, Bracci P M, Skibola C F. Genome-wide association study of follicular lymphoma identifies a risk locus at 6p21.32. Nat Genet. 2010 August; 42(8):661-4.
Conde L, Bracci P M, Halperin E, Skibola C F. A search for overlapping genetic susceptibility loci between non-Hodgkin lymphoma and autoimmune diseases. Genomics. 2011 July; 98(1):9-14.
Kang H M, Sul J H, Service S K, Zaitlen N A, Kong S Y, Freimer N B, Sabatti C, Eskin E Variance component model to account for sample structure in genome-wide association studies. Nat Genet. 2010 April; 42(4):348-54.
Modiano J F et al. (2005). Distinct prevalence of B-cell and T-cell lymphoproliferative diseases among dog breeds indicates heritable risk. Cancer Res, 65, 5654-5661. PMID: 15994938
Price A L, Patterson N J, Plenge R M, Weinblatt M E, Shadick N A, Reich D. Principal components analysis corrects for stratification in genome-wide association studies. Nat Genet. 2006 August; 38(8):904-9
Price A L, Zaitlen N A, Reich D, Patterson N. New approaches to population stratification in genome-wide association studies. Nat Rev Genet. 2010 July; 11(7):459-63.
Purcell S, Neale B, Todd-Brown K, Thomas L, Ferreira M A R, Bender D, Maller J, Sklar P, de Bakker P I W, Daly M J & Sham P C (2007) PLINK: a toolset for whole-genome association and population-based linkage analysis. American Journal of Human Genetics, 81.
Smedby K E, Foo J N, Skibola C F, Darabi H, Conde L, Hjalgrim H, Kumar V, Chang E T, Rothman N, Cerhan J R, Brooks-Wilson A R, Rehnberg E, Irwan I D, Ryder L P, Brown P N, Bracci P M, Agana L, Riby J, Cozen W, Davis S, Hartge P, Morton L M, Severson R K, Wang S S, Slager S L, Fredericksen Z S, Novak A J, Kay N E, Habermann T M, Armstrong B, Kricker A, Milliken S, Purdue M P, Vajdic C M, Boyle P, Lan Q, Zahm S H, Zhang Y, Zheng T, Leach S, Spinelli J J, Smith M T, Chanock S J, Padyukov L, Alfredsson L, Klareskog L, Glimelius B, Melbye M, Liu E T, Adami H O, Humphreys K, Liu J. GWAS of follicular lymphoma reveals allelic heterogeneity at 6p21.32 and suggests shared genetic susceptibility with diffuse large B-cell lymphoma. PLoS Genet. 2011 April; 7(4):e1001378.
Tamburini B A et al. (2009). Gene expression profiles of sporadic canine hemangiosarcoma are uniquely associated with breed. PLoS ONE, 4(5), e5549. PMCID: PMC2680013.
Wang S S, Abdou A M, Morton L M, Thomas R, Cerhan J R, Gao X, Cozen W, Rothman N, Davis S, Severson R K, Bernstein L, Hartge P, Carrington M. Human leukocyte antigen class I and II alleles in non-Hodgkin lymphoma etiology. Blood. 2010 Jun. 10; 115(23):4820-3.
Yang J, Lee S H, Goddard M E, Visscher P M. GCTA: a tool for genome-wide complex trait analysis. Am J Hum Genet. 2011 Jan. 7; 88(1):76-82.
Alizadeh, A. A., M. B. Eisen, et al. (2000). “Distinct types of diffuse large B-cell lymphoma identified by gene expression profiling.” Nature 403(6769): 503-511.
Anderson, J. R., J. O. Armitage, et al. (1998). “Epidemiology of the non-Hodgkin's lymphomas: distributions of the major subtypes differ by geographic locations. Non-Hodgkin's Lymphoma Classification Project.” Ann Oncol 9(7): 717-720.
Baraniskin, A., J. Kuhnhenn, et al. (2011). “Identification of microRNAs in the cerebrospinal fluid as marker for primary diffuse large B-cell lymphoma of the central nervous system.” Blood 117(11): 3140-3146.
Barrett, J. C., B. Fry, et al. (2005). “Haploview: analysis and visualization of LD and haplotype maps.” Bioinformatics 21(2): 263-265.
Carrillo, C., A. Hichami, et al. (2012). “Diacylglycerol-containing oleic acid induces increases in [Ca(2+)](i) via TRPC3/6 channels in human T-cells.” Biochim Biophys Acta 1821(4): 618-626.
Chang, K. C., G. C. Huang, et al. (2007). “Distribution patterns of dendritic cells and T cells in diffuse large B-cell lymphomas correlate with prognoses.” Clin Cancer Res 13(22 Pt 1): 6666-6672.
Chen, T. C., S. A. Lee, et al. (2009). “From midbody protein-protein interaction network construction to novel regulators in cytokinesis.” J Proteome Res 8(11): 4943-4953.
Conde, L., P. M. Bracci, et al. (2011). “A search for overlapping genetic susceptibility loci between non-Hodgkin lymphoma and autoimmune diseases.” Genomics 98(1): 9-14.
Conde, L., E. Halperin, et al. (2010). “Genome-wide association study of follicular lymphoma identifies a risk locus at 6p21.32.” Nat Genet 42(8): 661-664.
Damann, N., G. Owsianik, et al. (2009). “The calcium-conducting ion channel transient receptor potential canonical 6 is involved in macrophage inflammatory protein-2-induced migration of mouse neutrophils.” Acta Physiol (Oxf) 195(1): 3-11.
Drake, A. C., M. Khoury, et al. (2011). “Human CD34+CD133+ hematopoietic stem cells cultured with growth factors including Angpt15 efficiently engraft adult NOD-SCID Il2rgamma−/− (NSG) mice.” PLoS One 6(4): e18382.
Finney-Hayward, T. K., M. O. Popa, et al. (2010). “Expression of transient receptor potential C6 channels in human lung macrophages.” Am J Respir Cell Mol Biol 43(3): 296-304.
Frantz, A. M., A. L. Sarver, et al. (2013). “Molecular Profiling Reveals Prognostically Significant Subtypes of Canine Lymphoma.” Vet Pathol.
Glickman, L. G., N.; Thorpe, R. (2000). “The Golden Retriever Club of America National Health Survey 1998-1999.” (available at http://www.grca.org/healthsurvey.pdf).
Goldin, L. R., M. Bjorkholm, et al. (2009). “Highly increased familial risks for specific lymphoma subtypes.” Br J Haematol 146(1): 91-94.
Hasselblom, S., M. Sigurdadottir, et al. (2007). “The number of tumour-infiltrating TIA-1+ cytotoxic T cells but not FOXP3+ regulatory T cells predicts outcome in diffuse large B-cell lymphoma.” Br J Haematol 137(4): 364-373.
Khoury, M., A. Drake, et al. (2011). “Mesenchymal stem cells secreting angiopoietin-like-5 support efficient expansion of human hematopoietic stem cells without compromising their repopulating potential.” Stem Cells Dev 20(8): 1371-1381.
Lawrie, C. H., J. Chi, et al. (2009). “Expression of microRNAs in diffuse large B cell lymphoma is associated with immunophenotype, survival and transformation from follicular lymphoma.” J Cell Mol Med 13(7): 1248-1260.
Lindblad-Toh, K., C. M. Wade, et al. (2005). “Genome sequence, comparative analysis and haplotype structure of the domestic dog.” Nature 438(7069): 803-819.
Lippman, S. M., C. M. Spier, et al. (1990). “Tumor-infiltrating T-lymphocytes in B-cell diffuse large cell lymphoma related to disease course.” Mod Pathol 3(3): 361-367.
Modiano, J. F., M. Breen, et al. (2005). “Distinct B-cell and T-cell lymphoproliferative disease prevalence among dog breeds indicates heritable risk.” Cancer Res 65(13): 5654-5661.
Muris, J. J., C. J. Meijer, et al. (2004). “Prognostic significance of activated cytotoxic T-lymphocytes in primary nodal diffuse large B-cell lymphomas.” Leukemia 18(3): 589-596.
Navarro, A., A. Gaya, et al. (2008). “MicroRNA expression profiling in classic Hodgkin lymphoma.” Blood 111(5): 2825-2832.
Paoloni, M. C. and C. Khanna (2007). “Comparative oncology today.” Vet Clin North Am Small Anim Pract 37(6): 1023-1032; v.
Penel, N., S. Marreaud, et al. (2011). “Angiosarcoma: state of the art and perspectives.” Crit Rev Oncol Hematol 80(2): 257-263.
Price, A. L., N. A. Zaitlen, et al. (2010). “New approaches to population stratification in genome-wide association studies.” Nat Rev Genet 11(7): 459-463.
Priester, W. A. (1976). “Hepatic angiosarcomas in dogs: an excessive frequency as compared with man.” J Natl Cancer Inst 57(2): 451-454.
Purcell, S. “PLINK.”
Purcell, S., B. Neale, et al. (2007). “PLINK: a tool set for whole-genome association and population-based linkage analyses.” Am J Hum Genet 81(3): 559-575.
Ralfkiaer, U., P. H. Hagedorn, et al. (2011). “Diagnostic microRNA profiling in cutaneous T-cell lymphoma (CTCL).” Blood 118(22): 5891-5900.
Riemersma, S. A., J. J. Oudejans, et al. (2005). “High numbers of tumour-infiltrating activated cytotoxic T lymphocytes, and frequent loss of HLA class I and II expression, are features of aggressive B cell lymphomas of the brain and testis.” J Pathol 206(3): 328-336.
Rimsza, L. M., R. A. Roberts, et al. (2004). “Loss of MHC class II gene and protein expression in diffuse large B-cell lymphoma is related to decreased tumor immunosurveillance and poor patient survival regardless of other prognostic factors: a follow-up study from the Leukemia and Lymphoma Molecular Profiling Project.” Blood 103(11): 4251-4258.
Roedding, A. S., P. P. Li, et al. (2006). “Characterization of the transient receptor potential channels mediating lysophosphatidic acid-stimulated calcium mobilization in B lymphoblasts.” Life Sci 80(2): 89-97.
Romeo, S., W. Yin, et al. (2009). “Rare loss-of-function mutations in ANGPTL family members contribute to plasma triglyceride levels in humans.” J Clin Invest 119(1): 70-79.
Smedby, K. E., J. N. Foo, et al. (2011). “GWAS of follicular lymphoma reveals allelic heterogeneity at 6p21.32 and suggests shared genetic susceptibility with diffuse large B-cell lymphoma.” PLoS Genet 7(4): e1001378.
Tipton, A. R., K. Wang, et al. (2012). “Identification of novel mitosis regulators through data mining with human centromere/kinetochore proteins as group queries.” BMC Cell Biol 13: 15.
Tseng, P. H., H. P. Lin, et al. (2004). “The canonical transient receptor potential 6 channel as a putative phosphatidylinositol 3,4,5-trisphosphate-sensitive calcium entry system.” Biochemistry 43(37): 11701-11708.
Vaysse, A., A. Ratnakumar, et al. (2011). “Identification of genomic regions associated with phenotypic variation between dog breeds using selection mapping.” PLoS Genet 7(10): e1002316.
Yang, J., S. H. Lee, et al. (2011). “GCTA: a tool for genome-wide complex trait analysis.” Am J Hum Genet 88(1): 76-82.
Yao, H., F. Peng, et al. (2009). “Involvement of TRPC channels in CCL2-mediated neuroprotection against tat toxicity.” J Neurosci 29(6): 1657-1669.
Zeng, L., J. Dai, et al. (2003). “Identification of a novel human angiopoietin-like gene expressed mainly in heart.” J Hum Genet 48(3): 159-162.
Zhang, C. C., M. Kaba, et al. (2008). “Angiopoietin-like 5 and IGFBP2 stimulate ex vivo expansion of human cord blood hematopoietic stem cells as assayed by NOD/SCID transplantation.” Blood 111(7): 3415-3423.

All references recited herein are incorporated by reference herein in their entirety. The definitions and disclosures provided herein govern and supersede all others incorporated by reference. Although the invention herein has been described in connection with preferred embodiments thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

What is claimed is:

1. A method comprising
analyzing genomic DNA from a canine subject for the presence of a risk allele at a chromosome 5 marker that is BICF2G63035726 or BICF2G630183630, and
identifying a canine subject having risk allele at a chromosome 5 marker that is BICF2G63035726 or BICF2G630183630 as a subject (a) at elevated risk of developing a hematological cancer or (b) having an undiagnosed hematological cancer.

2. The method of claim 1, wherein the genomic DNA is obtained from white blood cells of the subject.

3. The method of claim 1 or 2, wherein the genomic DNA is analyzed using a single nucleotide polymorphism (SNP) array.

4. The method of claim 1 or 2, wherein the genomic DNA is analyzed using a bead array.

5. A method comprising
analyzing genomic DNA from a canine subject for the presence of a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1, and
identifying a canine subject having a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1 as a subject (a) at elevated risk of developing a hematological cancer or (b) having an undiagnosed hematological cancer.

6. The method of claim 5, wherein the genomic DNA is obtained from white blood cells of the subject.

7. The method of claim 5 or 6, wherein the mutation is in a regulatory region of the locus.

8. The method of claim 5 or 6, wherein the mutation is in a regulatory region of a locus selected from the group consisting of ANGPTL5, BIRC3, CD68, MYBBP1A, CHD3, CHRNB1, RANGRF, ZBTB4, and a locus comprising SEQ ID NO:1.

9. The method of claim 5 or 6, wherein the mutation is in a coding region of the locus.

10. The method of claim 5 or 6, wherein the mutation is in a coding region of a locus selected from the group consisting of ANGPTL5, KIAA1377 and TRPC6.

11. The method of claim 10, wherein the mutation is in a coding region of TRPC6.

12. A method comprising
analyzing, in a sample from a canine subject, an expression level of a locus selected from the group consisting of ANGPTL5, BIRC3, CD68, MYBBP1A, CHD3, CHRNB1, RANGRF, ZBTB4, and a locus comprising SEQ ID NO:1, and
identifying a canine subject having an altered expression level of a locus selected from the group consisting of ANGPTL5, BIRC3, CD68, MYBBP1A, CHD3, CHRNB1, RANGRF, ZBTB4, and a locus comprising SEQ ID NO:1 as compared to a control, as a subject (a) at elevated risk of developing a hematological cancer or (b) having an undiagnosed hematological cancer.

13. The method of claim 12, wherein the sample is a white blood cell sample from a canine subject.

14. The method of claim 12, wherein the sample is a tumor sample from a canine subject.

15. The method of any one of claims 12 to 14, wherein the control is a level of expression in a sample from a canine subject having lymphoma and negative for risk marker BICF2G63035726 and risk marker BICF2G630183630.

16. The method of any one of claims 12 to 15, wherein the altered expression level is
(a) a decreased expression level of ZBTB4, BIRC3 and/or ANGPTL5 compared to control, and/or
(b) an increased expression level of CD68, CHD3, CHRNB1, MYBBP1A and/or RANGRF compared to control.

17. The method of any one of claims 12 to 16, wherein the altered expression level is analyzed using an oligonucleotide array or RNA sequencing.

18. A method comprising
analyzing, in a sample from a canine subject, an expression level of a locus selected from the group consisting of TRPC6, KIAA1377, PIK3R6, ANGPTL5, HS3ST3B1, and BIRC3, and
identifying a canine subject having an altered expression level of a locus selected from the group consisting of TRPC6, KIAA1377, PIK3R6, ANGPTL5, HS3ST3B1, and BIRC3 as compared to a control, as a subject (a) at elevated risk of developing a hematological cancer or (b) having an undiagnosed hematological cancer.

19. The method of 18, wherein the altered expression level is
(a) a decreased expression level of TRPC6, KIAA1377, PIK3R6, ANGPTL5 and/or BIRC3 compared to control, and/or
(b) an increased expression level of HS3ST3B1 compared to control.

20. The method of claim 18 or 19, wherein the locus is TRPC6.

21. A method comprising
analyzing genomic DNA in a sample from a canine subject for presence of a mutation in a locus selected from the group consisting of TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, and
identifying a canine subject having a mutation in a locus selected from the group consisting of TRAF3, FBXW7, DOK6, RARS, JPH3, LRRN3, MLL2, OGT, POU3F4, SETD2, CACNA1G, DSCAML1, MLL, ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, as a subject (a) at elevated risk of developing a hematological cancer or (b) having an undiagnosed hematological cancer.

22. The method of claim 21, wherein the genomic DNA comprises a risk factor that is BICF2G63035726 or BICF2G630183630.

23. The method of claim 21 or 22, wherein the genomic DNA comprises a mutation in a locus selected from the group consisting of C11orf7, ANGPTL5, KIAA1377, TRPC6, NTN1, NTN3, STX8, WDR16, USP43, DHRS7C, GLP2R, BIRC3, CD68, MYBBP1A, CHD3, CHRNB1, RANGRF, ZBTB4, and a locus comprising SEQ ID NO:1.

24. The method of any one of claims 21 to 23, wherein the sample comprises
(a) a decreased expression level of ZBTB4, BIRC2 and/or ANGPTL5 compared to control, and/or
(b) an increased expression level of CD68, CHD3, CHRNB1, MYBBP1A and/or RANGRF compared to control.

25. The method of any one of claims 21 to 24, wherein the genomic DNA is obtained from white blood cells of the subject.

26. The method of any one of claims 21 to 25, wherein the mutation is in a coding region of the locus.

27. The method of any one of claims 21 to 26, wherein the mutation (a) is a frame shift mutation, (b) is a premature stop mutation, or (c) results an amino acid substitution.

28. The method of any one of claims 1 to 27, wherein the hematological cancer is a lymphoma or a hemangiosarcoma.

29. The method of claim 28, wherein the lymphoma is a B cell lymphoma.

30. A method comprising
analyzing genomic DNA in a sample from a subject for presence of a mutation in a locus selected from the group consisting of ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, or an orthologue of such a locus, and
identifying a subject having a mutation in a locus selected from the group consisting of ADD2, ARID1A, ARNT2, CAPN12, EED, ENSCAFG00000002808, ENSCAFG00000005301, ENSCAFG00000017000, ENSCAFG00000024393, ENSCAFG00000025839, ENSCAFG00000027866, L3MBTL2, LOC483566, MAPKBP1, NCAPH2, PPP6C, Q597P9_CANFA, SGIP1, XM_{—}533169.2, XM_{—}533289.2, XM_{—}541386.2, XM_{—}843895.1, and XM_{—}844292.1, or an orthologue of such a locus, as a subject (a) at elevated risk of developing a cancer or (b) having an undiagnosed cancer.

31. The method of claim 30, wherein the subject is a human subject.

32. The method of claim 30, wherein the subject is a canine subject.

33. The method of any one of claims 30 to 32, wherein the cancer is a hematological cancer.

34. The method of any one of claims 30 to 33, wherein the cancer is a lymphoma or a hemangiosarcoma.

35. The method of any one of claims 30 to 34, wherein the cancer is a B cell lymphoma.

36. The method of any one of claims 30 to 34, wherein the cancer is a hemangiosarcoma.

37. The method of any one of claims 30 to 32, wherein the cancer is angiosarcoma.

38. An isolated nucleic acid molecule comprising SEQ ID NO: 2.


SEQUENCE LISTING (25 sequences)

SEQ ID NO: 1 - DNA, 34013 bp, Canus lupus familiaris
tgtgcatggttggtccagatttggggtgtacgtgactaccactgtttgtgttatacatgatcaggagaatgaacaggaagaatatggaatccacttaacacatgtggtgtcgccaggaggagcatgattctgccagcattgactagacttatctctgatatgttttcaatctgcaaaactgagaagctagtaaatttgtatcaatttagactcttttcttcaatgagaagtgagttgaaggtgggtggatggagaccctctgttttcttctatccataaattgccaatatagaaatttatgcatatgtatttcatattcatttttttgatacaagaagaatgtcatgaactttatgtgggtcatagttttcttctttttttcccccttagcttgcaacttactaaaagagtaagttgttttgcaaagatcattaggtagattttttgtttgtttgtttgttttaatcgtgtatttctaatgttttgtgtgcagaaaatcttacttgctctttcccagctggaaatgatatctcttatatatttttatttttggatctcttttatgacatgtttcacttcctgttttagttcatagatacctaaatgtgttttatttttcctattagaatgtaaaacagccttgagaaatgatgttttatatactgtatttccagtatttataaatagaatatataagtaaattaaaaacttcccactttgaatagcaacagcaaaaaacatctatagtccctaaaagagaacagagatccagtggaaaatgttcctggaaaatgcagctctgctgttttctgggtactaaggagcctatgagttggtgtgacttcaggcaacatgttccttctaagttttttcacctgtaaaatgaaaatatgtctgcctcatagaactgatgagaaggtctaagttaggaaacacatagaactccaaggtgctatagaactgttaagaaaccacaccatatgcaaacgggaatgtggagctttcactctcacatttaacttagtttacagtcctcaggagaaatatataagccaaacattaagtatatggaagaacatcaacctcatagtaggaactcagaaccgaatttgctgaactgaattactgttaacttcaattatccaaaaagcttcccttagagagtcaaggatggaaggctctaagctggcacacaggaatagcgttattggatttgtgctcagatcatgattccacagagctttggatccatcccataagagagctgttgttggaaagcactgtgtaggattttacttagaggaatcagcattgactccttttttaatcgctcaacagaagtggcactcagaaattacctttgagttatccagctgctggaaatctaagagctgtccagaggcaagaaatacttaaaacttagagtggttaaggtaagtagatgctgaagacaattttcactagtgattgctgtcaactggagtgatagcagttggtaagacctattcctttattgaacttattcaatgcctacttgtgacagctgctagttagccacagggattcaggtgatggtaaggatgaaaatcccttccaacccttcagagtagctcttggaaggtactgtcacaagcgctggcatcaatgagtcctgcactagtggtggcctccacaccccaggatccatgaatgactcactctgacagaagcacctgtggagtaaaaccagcggtttccccaacatccaatatgaagcatcctaacattcaggagaagaatgatgatcactgagctttaaaaataaacttttattttccaaaaggaaagtcatggcttgattcccttgtcaattgacactgagtaatttccttcatctgtgtgcagttctctcttacattgtgtagcactttctagcttacactgcagctaactcttcttaaagggaagagaggctagaattaagcattgctggaaaaactatatatgaagatactcatgctttaataagaagtctaaaatagtctctaaaaagttgggtgactcattagtaacattctcttcccacggcctcctgccatataattttgacctgcagtgacctcccctgtacttttagatggacatcagcatctagtatttattatggtaccttggaaagatgccaaaagatcaaacagagcatgaaaatatttgtaggatggaaatgcattctatagactcttaagtatcactctaagaagttgatgtattgggtgaaataagaaaattgagattacttgcttacaaaacataggagttttcttttaaaattttgtaattcagaatggcttgcttacattttatggatttactggttatagcttcagatgaagagcatggaccttgaggtctgcctggttgtgttttttttttttttttagattttatttattcattcatgagagacagagagagagagagagagagaggcagagacacaagcagagggagaagaaggctccctgcagggagcccaatgtgggactcgatcccaggactccaagattacaccctgagccaaaggcagatgctcaaccactgagccacccaggcatccaagcctgcttgtgtttcaacatcatttttggtacttaccttatagattggtccattaggagaatcgaataaaataatgcatgtaaagcatttagtagagtgccatgcctacatcctaataatcatatataaatgtgcattcatggtgattttgttattcccagtcatttacatctaagtaatacgtttgttcattccctaccatatacctcagggagtgtatcattcaatgtcatactactttgttcaagaaacaatattctgtcagtgattggctgttaaagggagatttaaaggcttttggcctaattttaggacagttctgaaaaggcctttcagcctcagaacaatctgttggattagttcaagtctcatttgtgacttcatgttaatgggccaccataaatctaaccaagtaggatgccaaaaacagttgctatgctggatatggtgtctttgctagagcaggttaacactcttttgcctgcatgggatgtgagtcatcgatatagtaaatgcattcttgcgcttactcatcaagaaagaagattggaagctaatctcttatagtcacaagaaagggttaatagtctacaaaatcttacccaaggctacattaatcctactatttgtcataatttagttcaaagggacctgggttatcttaaaatactgcagaagaccattgttcccctatattaatgatgtcatgtcaatgggatgtgatgagtaagagctggcacatattctgaatgctttggtaaaatatttcccgctccaaaaggcagaaaaaacctgacatagttttctaggagaatttagtgacatgtaaagtgtttatgaatctaatggtctggggtgtacctttacatactttccagaaagtggggtggttattgctccttgcccctaaagaagaagcacaaagcttggtagggcttttttggggtttgtagtcaacacagtctccacatggggttactttaatgctcaatttatttattgatatgaaagccttttcaccctgagagattcagatcaagatcaggaaaggtcactgtagctggtctagtttgcaaaacgagcggttctgccacttgggctatacaacccagcagatcccataattctgatgttacctgtggtaaagagagatgcttggaccctgaactttatgggcaactaatattcgataaaggaggaaagactatccattggaagaaagacagtctcttcaataaatggtgctgggaaaattggacatccacatgcagaagaatgaaactagaccactctctttcaccatacacaaagataaactcaaaatggatgaaagatctaaatgtgagacaagattccatcaaaatcctagagaagaacacaggcaacaccctttttgaactcggccatagtaacttcttgcaagatacatccacaaaggcaaaagaaacaaaagcaaaaatgaactattgggacttcatcaagataagaagcttttgcacagcaaaggatacagtcaacaaaactcaaagacaacctacagaatgggagaagatatttgcaaatgacatatcagataaagggctagtttccaagatctataaagaacttattaaactcaacaccaaagaaacaaacaatccaatcatgaaatgggcaaaagacatgaacagaaatctcacagaggaagacatagacatggccaacatgcatatgagaaaatgctctgcatcacttgccatcagggaaatacaaatcaaaactacaatgagataccacctcacaccagtgagaatggggaaaattaacaaggcaggaaacaacaaatgttggagaggatgcggagaaaagggaaccctcttacactgttggtgggaatgtgaactggtgcagccactctggaaaactgtgtggaggttcctcaaagagttaaaaatagacctgccctacgacccagcaattgcactgttggggatttaccccaaagatacaaatgcaatgaaacgccgggacacctgcaccccgatgtttctagcagcaatggccactatagccaaactgtggaaggagcctcggtgtccaacgaaagatgaatggataaagaagatgtggtttatgtatacaatggaatattactcagctattagaaatgacaaatacccaccatttgcttcaacgtggatggaactggagggtattatgctgagtgaagtaagtcagttggagaaggacaaacattatatgttctcattcatttggggaatataaataatagtgaaagggaaaataagggaagggagaagaaatgtgtgggaaatatcagaaagggagacagaacgtaaagactgctaactctgggaaacgaactaggggtggtagaaggggaggagggcggggggtgggagtgaatgggtgacgggcactgggtgttattctgtatgttagtaaattgaacaccaataaaaaaaaaataaataaagagagatgctgtttggagtttctggaagaccaaagagagaaacacagtacagccccatagagttccagagtaaggacaagcctatataactatagacgattccttttatttttaagatgaaactgagatttatttattttttataaatttattttttattggtgttcaattagccaacatatagaataacacccagtgctcatcccgtcaagtgcccacctcagtgcccgtcatcacccagtcacccccaccccccacccactccttttccccacccctagttcgtttcccagttaggagtctttcatgctctgtctccctttctgatatttcccactaattttttctcctttcccctttattctctttcactattttttatattccccaaactatagacgattccaacagctctggatgctactgggctctaagagaaacccagtgtctcaccatggggcctatgtgaccacaattccagaactgcccatcagagtttggctgctgctagatataccaagtcataagatcaggtgaatgagaagctaccctatgatgggggcacatctcagggttggtgtagaagtcaccagtaggctaacatgataagtggcccagacactcatcacctaccactattgtggatgcttattttcagcctcaatttatatatgacaggtgatggagggtgccccgtgaccagctgatgaaggaaggaaacggttaacttgatcacaaggacggtggctaaaagtggacacccaactctctacacccccttgaggatggccttgaaaaccaggcaaggtgaaatcctccttgtgggcagagctgtgtgtggaggggaaattgtgcaagacaagggtgtgtgtgtgtccatagggcatggtgaaaagtctccctggctggctggggctgtacaggaagaaaactggagaaagtccaggtcaggaaggggcattaatttaccaggcacatggaataagcaggggttaattgcaagcctctgtccttggccactgccacacctgtgccatgactgcagccatgggaatggtacagtcatagggacggcccagtctgcccaccccaccccccaggccgaccaaccaaagactgaccattgtaagacagaagagggcccggcctccttccatgagtggtgcgtctgcatccagagcctctcacgggatcgaggatggccttcagatgggacctggccagacattctggcaagcttatttctccaccttatctttcttcccttacctccctactcttgagaacactccttcaataaatcactttaacaaggatcctgttcttgggctgttttctggatatctgacccaggccaccagtcatcagcatacagttggtgtttggagctgtgtgcatgagtgagatcccttaagagtgcagagtgacagcaacagtggctacaggacagtgagcggcaggactgctgagtcactgggaatgtgggacataaacctacatagaagccaagacaatgtttcaggaatgaaaaggcagttagagtgtcaatgggagaaagttccacaggtgctgtggtgacctcagcagattcccctggttctgggtgctgtggaaggtccagcaatcaccccagtgtctgacctcggggcaagggctctgatcctttgctgcaggtggaacaatgctgtagttcgtgctccagagttccctgggggaacaggtggaggctcagtatcacctgaagctgcatccttgtctcttcttcatctggaaacctcactcccttatagactcctcccgagagcatgggctcctagaaactgagcctggggttatgattctggggattcgttaaaggatcgctaagacaagaattcaaggaaaacaagtatcaaaatgtgtttttagcatatggaaactaggatgtaactagtaaaatggggcaaaagtcagagtagacagagaaatgaatagaaggtgagtcaaagaagtccatgggttcagacaagtgcaccaagcactttgttgtgaaaccaagcaaagcctttatggtagatagatttggaaacagggtaaaaacagatgcgtgttcatgtgtacacatgcgtgtacatgttgtaagacatggcattgagtgctctaagttgagagaaaataaccaacaagaaggaagaggttgataatccaggaaaagaggtggaaccagtatcactgaacacctgtgagatcttgtaaaagtttttgtgatgaaaaaccacaaacttgggggatgcctgggtggctcagtggttgagtatctgccttcggctcaggttgtgatctcagggtcctgggacagagtcccacattgtgctccctatgaggagcctgcttctccctctgcctatgtctctgcctctctctgtgtctctcataaataaagaaaatcttaaaaaatagtaaaaaagtaaaaagtaaaaaccacaaactcagaataaaggatgagtggcaacatattttattttatatttaataaaagtttttgctgttatagtctcacatactcagatgtatccaaacctcctgaatagataataatgataattttctgcaaaatggatgagcaggactttaaaaataatgggtttcttattgcctgacttgtgccatgtaaaatatatttcctgttgctagtgtcagatgtgataaactataaagcaagtgatgtgtgctattttaggtgtttgccaattatttaaaagcagactgataatataactactttgggatagataaaggtaacaaaaatctgagaaaaaggaactaagaaatttgatgttttaagttcagttaatataaactcacaggactacctgtggtaagggctacagaaagattagacaatgattcctgtctccatgatcttataattattgtgtactagacatccttatcttaaagtctaaaagttattcatagtagagcaggcttattttttaagattttatttatttatgagagacacacagagaggcagagacacaggcagaaggagaagcaggctccctgcaaaagcctgaggtgggactcgatctcggatcctgggatcacgtcctaagctgaagacagaagctcaaccactgagccacccaggcatccctagagcaggcatatcatgtctgtgatgtgaatacttgcctctcatttttccatctacaaatgccaactggctatgatggtcatgcaccttattttccacccccttcttgttgtttttcaaagactttgtccttttgactctgggaactcttacttatttgcccactttcttcttttggcttaattcaagaaatctgcaattgacaattcttcatcatactctgcttctcctatttctacaatatatgcaagtagatatttaggaattggctccaaagccaaggatgtagcattatcatcaagtcaggcctgtggcaagaattattaacttcctaggatcccccagttatctttatactccaggatgttgattcatttctgttttttccttggtacttccatcatttttttcatgtttcatcatgttttatttatagaagcaagtgtgaggagttaaaagcaactggttctcgagcttctggcaaaagcttgagctgaaatgttagctctgggctgtgcttgtgggtcagctgactcagatgttgccaggccctctggggtcatggcaaaggccacatgccttgtgagactctctactttcttcctggcatgactttcttcccccctttcttgcttgcacctcaataccagggtcagtcagtgtggtctggatcagagatgaccttcccaggatagtcaggtctgcaattgttctccttactttttccaataaaaatagtaattagttattgggaaactttgtaaagataatgcttctgttataggttcagctccaaatcttttattgctaagtgtttttactcactgtgttattaccaggagctaatgctgtcattcactgccaccgtgctccctttctaaatccctgggtttccagaagtcatcacgagttatattcagaaaattgtagtgataaaaatcttccatgtgtaaaaaaactcatgggaggtgacacccaaatacaaaccacagtgccaggaattctaagtttcagcttgccaaaaagccagacacatgttagggttactcaaaacgatgaacatggattttaaaattccacgttgactaatctaggtagatatggtcaccaaatagtcaagtaaaagatgtaataattctgcataacctgcttgtcttttgtactcctacctttgctctatcctttccccttcctcttttcctcatcaattttattgagaatgagaatgtcaaagtagagaaattaaactcaggttgtgagaaagccaggataacaatacttatattgggtaaactaggctttaaaacaaaaactgtaacaagagacaaagaagaacactaaagaataataaagggggcaatccaacaagaaggtctaacagttgtaattatttatgcttcaaacatgggagcacccaaatacacaaaacagttaataacaaacataaaggaaataattgatggtaatacaataatagtagggaactttaacaccttacttataacaacagattgataatccaaacagaaaatcaataaagaaacaatggctttgaatgacaccctggaccagatggatttaacagacatagtcagaacattccatcataatacagcagaatacattcttttcaagtgcacatggagcattctccagaaaatgtcatatattaggtcacaaaataaacctcaacatagtcaaaaagatcaaagtcataccatgcatattttctgatcacaatgcaataaaaatctggaaagagcacaagtacatgggttaaataacaggctgctaaacaataagtggatcaaccaggaatcaaagaagaaataaaaaattacatgggaacacatgaaaatcaaaacaaaatgttcccaaatctttgggatgcaagaaaaatgattccagcagcgaagtttatagccatacaggcctacttcaagaagcaagaaaaatctcaaataaacaacctaactttacacctaaaggagctagaaaaagagcaacaaacaaaactgaaagccatcagaaggaaggcagtaataaagattagagcagaaataaatgatatagaatcctaaaaaactccacaaaaataaaaacaaaaacccagtagttcaatgaaaccaggagctcgttcttcgaaaagatcaacaaatcgataaatctctagccagactcataataaaaaaagagagaagacccaaaataaacaaaatcacaaatgaaagaagagaagtaacagccaataccacagaaatacaaacaaccttaggggaatattataaaaaagctatatgccaataaattgcacagcctggaagaaatggataaattcctaaaagcctataacctcccaaaaatgaatcaggaagaaatagaaaatttgaacagactgccagcaatgaaattgagtcagtaattgaaaaactcccaacaaacagaagtccatggccagatgttttcacaggcaaattctaccaaacactagaagaagagttaatagttattcttctcaaattattccaaaaaatagaataaggaaaacctccaaattcattctatgaggccagcattaccttgataccaaagccagatacagactccactaagagaaagaaccaaaggccaatatccctgatgaatgcagatgtaaaaattctcaataaaatgttggcaagctgaattcaacattaaaaaaatgattcaccaaagctgacagtgagagctgagtgtttgctgtttttacaacagaattccaccccgtggctccatcagagatttctcaaaagccaggtctgctcagctaggccagggcaaagcctctcacccttactccttcactcgctaggcacatattcaagacaaagcaaagcctcagtagagcggttttgtatttcagggactgtagaatgccttgtcagaaattgtttaaaaggaagatttggtgtccatcgaaagatgaatggataaagaagatgtggtctatgtatacaatggaatattactcagccattagaaatgacaaataccatttgtttcaatgtagatggaactggagggttccatcaacattgctgagtggaataagtcaatcagagaaggacaaacattatatggtctcattcatttggggaatataaaaaatagtgaaagggaataaaggggaaagaagaaaaaatgagtgggaaatatcagaaagggagagagaacatgagagactcctaactctgggaaacgaacaaggggtggtggtataaagggaggtgggtgggggtgggggtgactgggtgatgggcactgagatgggcccttgaagggatgagcactgggtgttattctatatgttggcaaattgaacaccaataaaaaattttaaaaaatgattcaccccaatcaagtaggatttattctcaggatgcaagggtggttcaatacctgcaaattaatcaacatgatacattacatgaataaaagaaatgataaaaaccttatgatcatctcaatagatgcagaaaaagcatttggcaaagtatgacatgcattcatgataaaaaccttcaacaaagtcgatttagagggaccaagcctcaatataataaagactttatatgaaaaacccacaggtaacatcagactcaatggtgaaaaatgtaaagctttcccctaaagatcaagaacaaaacaaggatgtccactcgtaccactgttgttcaacacagtactggaagtcctagcctcagcagtcagacaacaaaggaaataaaagtcatccaaattggtaatgaagaagtaaactttcactgtttgcagaagacatgatactatagatagaaaatattaaagattacaccaaaaaacccatactagatctgatgaattcagtaaggttgcaggatacataatcaatgtacagaaatctgttgcatttctatgcatttataatgaagcaacagaaagagaaattaagaaaacaatcccatttacaattgcaccgaaataatacatcttctaggaataaacttaacgaaagaggtgaaaaacctatactctgaaaactataaaacactgatgaaagaaattcatctttctagatatgtctcctgaggcagggtaaataaaagcaaaattaatctattgagactacatcaaaataaaaatctatacagtgaaggaaacaatcagcaaaactaaaaggcaacctacagaatgggagaagatatttgcaaatgaaatatctgataaagggttggtatccaaaatatataaagatcttatataactcagcagccaaaaattgaataatccaattaaaaaatgggtggaagacttgaaatgacacttttccaaagaggacatacacatggccaacaggcacatgaaaagatgctccacatcactcaccatcagggaaatgcaaatcaaaactacagtgagatcccacctcacacctgtcagaaatgttaaaattaacaacacaggaaacaacagatgttcacaaggatgaggagaaaggggaaccatctttcactgttggtgggaatgcaaactggtgcacccactctggaaaactgtgtggaggttcctcaaaaaattaaaaatagaactaccttatgacccagcaaatgtactactaggtatttacccaaaggaaagaaaaatactgattcaaatgtttataatagcaatgtccatgatagccaaactatggagagagcccagatttccatcaacagatgaatggataaagaagctgtggtataaatacaatggaatatcacttctgtcataaaaaaagaatgtgatcctattatttgcagtgatgtggatggaactagatggtattatgctaagcgaaataagtcagagaaagacaaataccatatgatttcactcacatctggaatttaagaaagaaaacatataagcatatgggaaaggagtaggaaaaaaaggagagagagaaacaaagcataagaggctcttagcaataggaaataaataggaaacaaataggaggattgcttgaggggacctgggtgggggatgagctagaaggggtatgggcattaaggagggtgttgtgatgatgagcactgggtgttacatattagtgatgatcactgaattctactcctgaaaccaatattgcactgtatgttaactaaatagaatttaaataaaaatttgaagaaaataaaaataaaaaataaaaataaataaaaattatgtttttgcaatcaaaagaaaagaaaatgttatctttatacctactacctcaaacacattttaaagtagatttggatagatcattatagcattttgcccctacattagaggtgttttatcttaatatttaactcaatttggaatgtgggcagatggaagaagaggactctctaaggtcaatatgaaggatatggaagctggatcactttaaagaactggtagagaatgtcagaagttaagaaaaaaagtgtcatctggtcaatactgccatagctgctatgaaaaatgcttttgttggataactaggaagtcttggtgatcttaggtagcaaagtttcatgttaaggctcaatttagagtcccttgaaatctcaggctatcttcattaaactatgtcttcagcacatgctcttgtgctcagtcacaaaattcatgaacatccaaacagactgacctgctgaagaacccataaaaaagactatagagaaggtgaataaattgcaaaatttttgccagcaaaagccagaaacacattccaaataatacatgatagtacttatgtatgtcatgcgtatcattgactcagtttttctggatattattttgaatttctgtacatgttctcatggagagaacacattttccagatacagaggttctgaaggagtactctggagatctgttcaccatgtaagtcacatagaaacagaaatagtggttttcatagcaattcaaactgtcgggttagaaaaaactaacatgtaattttaaaggacactaatacacatgagttttgttacaaaacgtagactgatgggacttttgatttcactcttcaaagtatggaaaactttattaatttggttaacatatcctcatctctttggacggaaaaatctgtaacaactaagaacctcaaatacagcatttcattgcctccttgactcatgttttgaagatgtactaattgcttcttgtcctttacttctttaaaaaaaaaaaaccaaggagggacgcttgatggaaaaagaacaattggtaaggttattagttgtgaatgacttagagaaaaacaaaaaccaaaagacgtttgtgttgtttgataattttccctaaataacattttcttggtacatatatttttctaattatttgttttgttacatattttgaaaatatttcagccttttgaaggaaaattagaaaacatcttgttttctcagatccaggtatgtgagtctaagatgcctggaatttttaattagctggaatgaaggaaaaacatcgatatatagtatgaacttgccttttaatgaaattgatgacttttttttgctttgattacatactcatcattttattattaattttatttttagaaaatagtgggagttggaaaaatattaagaatatgttgacttatgactaaggtagtggtctgtgggcctatctgacaaggaagataatatcagggtgttccagaatgcatcctacttgtctagcattgacataggtacaaaaataaaatgttcaaataaggcttttttttccttcaatagcttttaatatttaaatagtaccatcctactatcatttaagagtttgaggtatttatgttattttattaaaaaaaataaatgttaggaataagttctctagaaattgaggaaaggagatcagtgtacatgggagtgtttctcaaacttcattaaggaggctgatcttgacttggctctagcttccagagggatgagtagaaggatttagttggtggaaggagaagtagggtgaaaacagacactgctgactcagcactcctctctcactcatggctgcccacaatgggcagctggtagatgtcccgtcaaattaaagtggacaaagtgttttaccaccattttcctaagccttgattgaaggtttttgttccaagtaggtggtgggattttgagtgtggaatttacgtaagctattaagagaatagtgtaaaacaggtagaagcccattctattaaaatttcttttgctcttcaggtgtgtgctattttcataaacgttgctcacatctattacaaacatttctaaacacatatttaaatatattttatataaataaatatggtcaagaaatcatcatgaatgttattatctggtggctggtgataatccttctatcagttttttcccctttgaaaattcctgtaagtcaaagatgaaatttttaaaaactatcagaaaaagtgatgtcagcaaacaaaatggactggataattccaaaggcccatccctcagagaaatactaaaatatgaagcaaaaactgtcagaagcaactttgtaagaactctggaaaatagccaaaagtttacagaaagcaagaaaatgctggataagaaaggagagataatggtggtgacagttttaacataataggaaagctttgtcaaattttgcttgctcttgcctcacttgccttcctggtctggtggtgatcttgaagatagtagcctgagttcccagtgtggactctggtctctggtcctggagggagcagaaaagatcatattcttaaagaactatgttttgtctgttttgatctatctggagatgacctaaaggatggatgtaaggtgcttgattttgttccacccaacttggaactctcttgggatcaaagagtggctatgcagagggcattccttgaaaacattgtgaggcaatagaacaatgtgctatagcctggggcaaaagactgcatttaagccaaaaaatgacactccaacagtccaagagtccagttactccaagagaaactggggagagagtttctttgggagattaagccattgagaagtgcctgtataaaccagagaatctagaaagctgtgtgcatagccaggacagaatgctttctcagagagaccaggcaacacccagagctttcatctatggccattctttgggctcacagaaagcataaagtgggggctaatgcagacttgtaaacagcctggctaagcactgaaagaaccaatctgcaaacattgggagaggctctttttctcctctcttccctctccttcccttcccttcccttcccttcccttcccttccctctccttccttcctctcctctcctttcctttcctttcctctcctcccctcttctttctttctttctttctttctttctttctttctttctttctttctttctttctttccttcttattttctttcttccttcacccaggcattcaaggcaatctctgtcaacactagctgactataagccaaaggtcagaaacttcagaaactacatccaacaaataatacagactttacagaaatagtttagaaaagtaacaaaagaaacaaaccagtacagcctacagcaagcaaaaataaaataaattacccaaggagggggaagaatctgatttccagttatcatattataatattcaaatatcagttttccacaaaaaattacaatattcaaagaaaaaaagaaagtagaacctatccacagaggaaattaacagaaattgtcatttaggaagcacagatattggacatatgagacaaagattttaaaccaactgacctactggactagcagagttaaaggaaaccatagacaaagaggtagatatcagtaaaatgaagtctcaacaaatagagaatatcaatagaaattataaaaatgatctcaacagaaatgctggacctgaaaagtacaataccagaaacgaagaattcaatagtaggtttcagtagaatatttgagcaggtagaagaaagaacaagcaaacttgaagacagatcaattaaaattacccagtgtggggagaagaaagaagagggaatggagaaaaagacatcatcaaacatacaaacatactcatcaggtgagttccaagaaagaggacagagagaaagggacagaaagaatatttgaaataataatggccccaaattcctccaagttgaacatgaatctacacatctgagaggctcaacaaactttaaggagtataaacatgaggataaccattctgagacatactataatttaactgttaaaaatcaaaaatggagaatcttgaaagcagtgagataagcaatgcattatatatgagtgattctacatgaaattaacaggtaatttcccagcacaatccatggaattagaaggcatggaatgacatattaaagtgttgaaagaaaaaaatctgccaaccaaaaattgtatatctggcaaaactatccttcaaattgaaggaaactctagggcatttcccaataaagagaagctgaggtagttcatcgctagtagacctgctctatggaaatactaaagggaatccttcaggtggaaatgaaagaacaccagatgatgacttgaaggcatacaaagaaatgaggaacactgggaaagttcactagataggtaaatacaagcgatagcatgcccagttttgccacttctgtttagctttataccgatgttctagccagagcgattaggcaagcaaaagaaacaaagacatccaaattggagaggaataagtaaaactatttatatccacagatgatcttatatgtagaaagtccgacagaatccacaaaatacaattagggctaataaattgtgaaaaactgcaggatataagatcaatacacaaaagtcagtcttatttttatacactagcaatgaattgtccacgaataaaaccaagaagacttacaataacatccataatagtgaaatacctataaataaatgtaacaaaggtgaaagacctaaataaatggagacaacctgtgttaatggattagaagacttaatattcatgtgatctacagatttcagtgtgatctgtatcaaaattccaatagcctttgtccccccagaaatagccaatcctcaaatttatatggtattggaagaagtaaaaacaattttgagaaggagcacagttggaggactctcacttcctgatttcaaagtttaataaaaagctactttagttaaaacaatgtggtgctgacctaaagaaagacataaaaatctgtagaatagaatagaaagcccagagataattcttcacacatatggttaactaattttcaacaagagtgccaagatcatcatacagggaatgaatagtttcctcagcagatgatactggggcaaatggacatgttaaagaatacagttgcacccctacctcatactatatacaaaattaactcattatctacatttagataagcaacctaaatgtaggagctaatgttataaaacccttagaagaagaggtgaatcttcatgactgaggatttggcaatggattattagataattcaaaagcataaataatgaaagaaaaagcaaatgaactaaacaccaccaaaatttaaaacgtttgtgcattgtaggacattgtagagaaagtgaaaagacaacctacagaatgtaagaaaatatttgcaaaacagatatttgaacagaatatattcattcagaatatataaagagctcaactcaacaacaaaaaggcacacaacctgattaaaaatggacaaaggagctgagtagacatatatttgaggaaagtatacaaatggctaacaagtatataaagtatgtttaacattattaggcatgaggtaaatgcaaattaaaaccatgatagagatcactttacacccactgggatgtctataatagaaaaagtgttgtcaaggatgtggagaatttggagcccttgcatactgttagtgagaatgtaaaatggtgcagccactatggaaagtcatttagtggttcctcaaaaagttaaacatagaactatcaaataacccagcaattttacttgtaggcatactcaccacccctgaaatagaaaagagatactcaagagggcctggatggctcagtcagttaagtgtctgactcttgattttattagttcaggtcatgatctcagggtcatgatattgagccttgcagtggctccatgctctctctccctctctgtccctctccctccccacccctgctcacacttgcatgtgctctgtgtctctaaaaaaatcaaaatttaaaaatttaaggaaagaaaagaggtatcaaacaaatacctgtactgggatcttcacagcagtacttttcacaatagacaaaatgtggataaaacaaaatgcattaatggatgaaaggaccaccaatgtggtaggtacataggattggttattatttggcaataaaaagaatgaagtatctgtatatgttatactgtaaacatctcagaaacattatgctaagtgcaaaaagctggacacaaaaggtcttacagtgtatggctccatttaaaaaaaaattttattggagttcaatttgccaacatagagcataacccccagtgctcatcccgccaagtgcccccctcagtgcccatcacccagttaccccaacccctgccaacctccccttccactaccccttgttcgttttccagagttaggtgtcatgttttgtcaccctcactgatactttcactcattttctctcctttatttaaaaaataaatttattttttataggtgtgcaatttgtcaacatatagaataacacccagtgctcatcccatcaagcgcccacctcagtgcccgccacccagccagccccacccccatccccctccccttccaccacacctagttcatttcccagagttaggagtctttcatgttctgtctcccttcctggtatttcccacttatttttttcctttcccctttattccctttcactattttttatattccccaaataaatgagaccatataatgtttgtccttctccgattcacttatttcactcagcataataccacccagttccatccacatcaaagcaaatggtgggtatttgtcatttctaatggctgagtaatattctattgtatacataaaccacatcttctttatccattcatctatcgatagacactgaggctccttccacagtttggctgttatggacattgctgctataaacatcggggtgcagatgtcctggcatttcactgcatctgtatctttggggtaaatccccagcagtgcaattgctgggtcatagggcaggtctatttttaactctttgaggaacctccacacagttttccagagtggctgcaccagttcacattcccaccaacagtgcaagagggttcccctttctccacatcctctccaacatttgtggtttcctgccttgttaattttccccattctcactggtgtgaggtggtatctcattgtggttttgatttgtatttccctgatggcaagtgatgcagagaattttctcatgtgcttgttggccatgtctatgtcttgctctgtgagatttctgttcatgtcttttgcccatttcatgattgtattgtttgtttctttgctgttgagtttcgtaaattctttatagatcttggatactagccctttacctgataggtcatttgcaactatcttctcccattctgtaggttgtcttttagttgtgttgactgtttcttttgctgtgcagaagctttttatcttgatgaagtcccaagaattcatttttgcttttgtttcccttgccttcatggatttatcttgcaagaagttgctgtggccaagttcaaaaagggtattgcctgtgttttctaggattttgatggaatcctgtctcacatttagatctctcatccattttgagtttatctttgcgtatggtataggacaatggccagtttcattcttctgcatgtgaatgtccaattttcccagcaccatttattgaagagactgtcttttttccagtagatggtctttcctgctttatcgaatattagttaaccataaagttgagggtccacttctggattctctattctgttccattgatctatgtgtctgtttttgtgccagtaccacactgtcttgatgaccacagctttgtagtacatcctgtaatctggcattgtgatgcccccagatatggttttcctttttaatattcccctggctattcggggtcttttctgattccacacaaatctccagatgatttgttccaactctctgaagaaagtccatggtattctgatagggattgcattaaatgtgtaaattgccttgggtagcattgacattttcacaatattaattcttccaatccatgagcatggaatattttcacatctctttctgtcttcctcaatttctttcagaagtattctgtagttcttagggtataaatcctttacctctttggttaggtttattcctaggtatcttatgcttttgggtgtaattgtaagtgggattgactccttaatttctttttcttcagtctcattgttagtgtagagaaatgtcactgacttttgggaatttattttgtatcctgccgcactgccaaattgctgtatgagttctagcaatcctggggtggagtctttgggttttctatgtacagattcatgtcatctgcaaagagggagagtttgacttcttctttgccaatttgaatgctttttatttctttttgttgtctgattgctgaggctaggacttctagtacttttttgaagagcagtgatgagagtgggcatccctgtcatgtccctgatcttaggggaaaggctcccagtgtttctccattgagaattatatttgctgtgggcttttcgtagatggcttttaagatgctgaggaatgttccctctatctctacactctgaagagttttgatcaggaacggatgctgtattttgtcaaatgctttctctgcatctattgagaggatcatatggttcttgttttttctcttgctgatatgatcaatcacattgattgctttacaagtgttgaaccagccttgcatcccggggataaatcctacttggtcatgatgaataatcttcttaatgtactgttggatcctattggctagtatcttgttgagaatttttgcatctgtgttcatcagggatattggtctataattctccttttttggtggggtctttggttttggaatcaaggtgatgctggttatggctccatttatatgaaatacacagaattggtaaattaacggttacaaaatcagattggcagggtgtcaggggctgggatgagggagaacacagagtgcctgcctactatcacaaggtttcctttttggctcacaaactgttttggaatttgatatggatagtcgcacaatgttatgaatatattaaatgccattcactttaaaatggtttattttacgttatgaaaatttcacctcaataagaaatcactggaccctgtgttccaggcaaaacagaaagagaaaatagagaaatgaattattttgattagcactgtaattaaaattacgctactcagataagggcacattcagtcattctgtaatttatttcttacaacttcactgaatgtctaattatcttgaaataaaaaaattaattgaaaagattattggagtttgttgaaaagctgaaaggtcgtttaaatggggagatataatcaataggtacttgggacttagtccactcttctattttctattagtttaataatagacacatactacttactgtgtggtaggcacagtgctaagtgctttacaaataataactctgtaagcagctattcttatagccacttcctggaacagtgtccccagtgctggcaacagtgctcggcacttggtaaatgctacatagataagtgttggaggattagatggtactctttccaagttgcctcattgcctttgtttcagtatctaaattcctagataaaatttctttcatctagatgactctagatcttcagatatttggcttcttctgaaagtagaactttatatgtgaagctgggagcagatgctttgattgggaggggagcccaggaagcactagagaaggtgcatagataatgggctggggagggagaagaagcccacactatggtaagtccttgaagttgaggctggcacagtttttcaagaatctctgaggaacatacagaatgggactctccatctgaagtacaggaggttggggcacttctctgccacttcccatgcctccctgcctgagagctgcccatactcctggactttgcttttgtggggctgagcaagctctttggctctgaacaggctttggtgtggaactgtggaggtggagtgctttagatggcatgttggcgcttttacagacaggtggtcccaactgcagcgggaatcagctgcggtgtgttgatgagatgcaaggcagcagaagcatttgctacatctgatagatatgatctttaatttagtgaacaacttttaaatgataatcaaagaaaatatcataaattaaatgcacaaatgtaacagttacaaactgagttgttagatatctaaaagccagcattcattttcagaagatagatcactgtctcaatgtgctgtagaaatatttatggtgatagacatgatatgtacaaactgtctacccttaggtcatggaaaccttctgctgttggctttcccttccacccactcccccagactcaagctctttggtttacagctgactcttctcaagcatctgtgtttactgaacagcagttcaatattcccataaatggtctgtgcttctctagaaaaatgcttgtttttcagcatcatcactggtgtgtattgtgattgctcaaaatttaatggaagggttatgacttcggtttatactctggtatcattagtgggccatgacccacctggagtttgtaatacagcattttctcagaaatcagtcacaggacctgggattattaaaagagaatgaaaacacatctttatcaaagaccatatcggtgcagatggaaagctatgtctatttttgaacattagctttttttcactaatttttaatcgcagactacttttccctgaatataacaagtagaaagtagttaattttcctgtagaaaggtctctgaagatttcactcatcctttcttgtctctctaatctcccaaatgactggaaataatgaaggaagagatgctggtactcctctggggtaatgatctcatgacttccaagggtgggtttgccgtcataatagttggggacaaaagtcagggtggtaactgggggtcgaataagatctggcaatcttgagaatgtgaagtaatttggaatatagcatgctcacagagtgggtattttatcctgttgggtagaaaagaccatcaggtgtgctggtgtctgttactcttgggagcccctagtttaaagactaggaggctagctcagagttgaggagatgcctttttttcaagagtaagaataattttgattttcagtatttccagtggagcagatccctccagaaaagtcgtgagagctgcatgtcaattgctgttatcccaatggtatttagaacattttcttgattaattacaagaaaatttggcctgggagggggctgcatcctgctgctttcaggagccatggcctgggagcacaattccagcagtgcaggccctggatcccagggtgctaaggggacacagcccaggatcctgcactcctctggggataggcagaggcagggagagcacaggacagtgagggctttcctgctgctgggtgcccccaagttgtgcaggtcagtgacccccgccctgggagcatccaggccagtgcagactgggagactgcggtagtcaccgcagggagcagactacagggctagggacctggccgccaccagtggtgttgttcttctttgtttcaccctgtgcctgggagaggcagggtgtcagggaacaggggtctcacgaggtaaacagctcccactgagcccggcacctagcagggggcacggtagctcccaggtacacacacctgagaaccagcacaacaggcccctcccccagaagaccagctggatggacaggggaagagcaagttcctgactaagcagcactaggaaggtccaggggaagttgagggatttacagtacatagaaccagaggttacccctccttttttcctctttttttcttcctttttccagtacaacttgtttctatatcagactgtaaatttccattttttttcttttttcccaccttaactacaatattttaccacctattcatttttaagattcttcctttttgacttcaatatttctacaattacaggtcctagatatattttccacttctagattcccttcaacgtgctcaccataattttgggagatatacaagatatattttttgttttgttttgtgttttgtgttttctctgcctcatgttgttctacaatggcagaagtttttttttaataataataaatttattttttattggtgttcaatttgccaacatacagaataacacccagtgctcatcctgtcaagtgcccccctcagtgcccatcacccattcacccccatcccccgcccttctccccttccatcacccctggttcgtttcccagagttaggagtctttatgttctgtctccctttctgacattagaaatgacaaataccgaccatttgcttcaacgtggatggaaatggaaggtattatgctgagtgaagtaagtcaatcggagaaggacaaacattgtatgttctcactcatttggggaatataaataatagtgaaagggaatataagggaagggagaagaactgtgtgggaaatacaatggcagaagttaataccttcaaaaacatgaccagtatgcacccagaaccaagtggtatactgtgctggttcattctgtgagattcctcattcccattctgcccccctgttttatctcatttatgttttggtggtcaatgttggggccttctacaagtatttctgttttatataaatttggaactgagtgtcttctaatatacagaacttaatatactcagaaacaagaggatcaccctctagaaccccccaggtagactacattctcccactactacaacttcgtcaccaccaccatctcccagtaccccccccccttgaattcttctcctttttttcttttttctttttcttttctttttttttctattctttaggattcctggccttttattttttactactttgttttataattaggtttcactttagtggtccttttgttttatttcattctgatctttgttttcaatttctggtctctgaccctggcagaatcatctagggtgaaatttacttaggtcatggttgatattcttgatgcagcccactcatacagccattctgcactgagcaaagtgactagaaagaactcaccacaaaagaaagaatcagaaatagtactctctgccacagagttgcagaatttggattacaattcaacgtcagaaaaccaattctgaagcacaattataaagctactggtggctatggaaaaaagcataaaagaatcaagagacttcatgactgcagaatttagatctaatcaggccaaaattaaagatcaattaaatgagatgcaatccaaactggaggtcctaacgatgaaggttaatgaggtagaaggagtgagtgacatagaaaacaagttgatggcaaggaaggaaactgagggaaaaagagaaaaacaagagactatgaagtaaggttaagggaaataaatgacagcctcagaaggaaaaaaatctacatatcattggggttccagagggcgccgaaagagacaggggaccagaaagtgtatttgaacaaatcatagctgagaacttccctaattaggggagggaaacaggcattcagatccaggaaatagagaggtccccccctaaaatcattaaaaactgttcaacaccttgacatgtaacagtgaaacttgcatattccaaagataaagaaaaaacccttaaagtggcaagagacaagcgatccctaacttacatggggagggatattagattaacagcggacctttccacagagacctggcaggccagaaaggactggaatgatatattcagggtactaaatgaaaagaacaggcacccaagaatactttatctagcagggctctcattcagaatagaaggggagataaagagcttccaagataggcagaaactgaaagaatatgtgaccaccaaaccagctctgcaagaaagattaagggggattctgtaaaagaaagaagtccaaagaaataatccacaaaaacacagactgaataggtattataatgacacaaaattcatatctttcagtagtaactctgaacgtgaatgagctaaatgatcccatcacaagacacagggtttcatactggataaaaaagcaagacccatctatttgctgtatacaagagactcattttagacagaagggcacctaaagcctgaaaataaaaggttggagaaccatttaccattcaaatggtcctcaaaaaaatgctggggtagcaatccttatatcagataaattaaagtttatcccaaagactgtagtaagagatgaagaggaacactatatcgtacttaaagggtctatccaacaagaggacctaacaatcctcaatatatatgccccgaatgcaggagctgccaagtatattaatcaattaataaccaaagttaagccatacttaaataacaatacacttatacttggagacttgaacatggcactttctctaattaatcttcaaaacacaacatctccaaagaaacaagaactttaaatgatacactggaccagatggatttcacagatatctacagaactttacatccaaacgcaactgaatacacattcttctcaagtgtacatggaactttcttcagaatagaccacatactgggtcacaaatcacgtcttaaccaatactaaaagattgggattgtcccctgcatattttcagaccacaatgctttgaaacttgaacttaatcacaagaagaaatttggaagaaactcaaacatgggaaggttaggagcatccttctaaaagatgaaagggtcaaccaagaaattagagaagaattaaaagattcacagaaagtaatgaaaatgaagaaacaactattcaaaatatttgggatacagcaacaacagtcctaagagggaaatacatcgcattacagcatccctcaaaattgggaaaactcaaatacacaagctaacctcacacctaaggaactggagaaagaacagcagataacacctatgccaagcagaagagagtttataaatagtcgagcagaactcaatgaaatagaggcccgaagaactgtagaacagatcaacaaaaccaggagttggttctttgaaagaattaataagatagataaaccattagccagccttattaaaaatgaaaaagaaaagactcaaattaataaaatcatgaatgaagaaggagagatcacaaccaataccaaggaaatacaaattatttaaaaaacatattataagcagctacacaccaataaattaggcaatctagaagaaatgaatgcatttctggaaaaccacaaattaccaaacctggaacaggaagaaatagaaaacctgaacaggcctataaccaaggaggaaattgaagcagtcatcaaaaacctcccaagacacaaaagtccagggccagatgggtttgcaggcagattctatcaaacatttaaagaagaaacaatacctattctactaaagctgttctgaaagatagaaagggatggaatacttccaaactcattttatgaggccagcatcaccttaattccaaaaccagacaaagaccccaccaaaaaggagaattatagaccaatttccctgatgaacacagatgcaaaaattctcaacaagatactagccaataggatccaacagtacattaagaagattattcacgatgaccaagtgggatttatcctcaggatgcaaggctggttcaacactcgtaaagcaatcaatgatatagatcaaataaacaagagaaaaataagaaccatatgatcctctcaatagatgcagagaaagcatttgacaaaatacagcatccattcctgatcaaaactcttcagaatgtagggaatagagggaacatatctcagcatcttaaaagccatttacgaaaagcccacaacaaatataattctcaatggggaaacactgggagcctttcccttatgatcaggaatacaacagggaggtccactctcaccactgttattcaacatagtactagaagtgctcgcctcagcaatactgtcttgaggatcacagatttgtagtataacttgaaatccagcattgtgatgcccccggctctggttttctttttcaatattcccctggctattcgcgtttttttctgatttcacacaaatcttgagattattcattccaactctatgaagaaagtccatggtattttgatagggcttgcattgaatgtatacattgccctgggtagcattgacattttcacaatattaattctgccaatccatgagcatggaatatttttccatctctttgagtcttcctcaatttctttcagaagtgttctatagtttttagggtagatcctttacctctttggttaggtttattcctaggtatcctatgcttttgggtgcaattgtaaatgggattgactccttaatttctctttcttcagtctcattgttagtgtagagaaatgccactgacttctggtcattgattttgtatcctgccacactgccaaattgctgtatgagttttagctatcgatggggtggagtcttttgggttttctacatacagtatcacgtcatcttcaaggagggagagtttgacttcttctttgccaatttgaatgccttttatttctttttgttgcctgattggtattggcacaaaaacaaacatggatcaatggaacagaatagagaacccagaaatgggccctcaactctatggtcaactgttattcgacaagcaggaaagactatctgctggaaaaaggacagtctcttcaataaacggtactgggaaatttggacatccacatgctgaagaaggaaactaaagcattctcttataccatagacaaagataagctcaaaatggatgaaagatctaaatgtgagacaggaatacattaaaatgctagaggagaacacaggcaacaccctttgtgaacttggcctcagtaacttcttgcaagatacatcctgaaggcaagggaaactgaagcaaaaatgaacttgggacttaatcaggatgaaaagcttctgcacagcaaaagaaacagtcaacacaactaaaagacaatctacagaatgggagaagatagttgcaaatgacctatcagttaaagcgctagtatccaaagatctataaagaacttatgaaactcaacagcaaagaaacaaacaatccaattatgaaatgggcaaaagacatgaacagaaatttcaccaaagaagtcatacacatggccagcaagcacatgagaaaatgttccacatcagttgccatcagggaaatacaaatcaaaaccacaatgagatcccacctcacaccagtgagaatggcgaaaattaacaagacaggaaacaacaaatgttggagaggatgtagagaaaggggaaccctcttgcactgttagtgggaatgtgatctggtgcagccactctggaaaactgtgtggaggttcttcaaagagttaaaagttgagctacccttcaatccagcaattgcactgctggcgatttaccccaaagatacagatgcagtgaaacgccaggacatctgcaccccgatgtttatagcagcaatgtccacaatagccaaactgtggaaggagcctcagtgtctatcgataggtgaatggataaagaagatatggtatatatatataaaatattaatatattatgaaatattatatttatatataatattttatatagtaaaatatatgaataatgaaatattcataattaataattaataataatgataaattttcatttcaacaatgaaataaaaaaatgaaatattatatatatacatatatataaaatgaaatattactcggccattagaaatgacaaatacctaccatttgcttcaacgtggttggaactggaaggtattatgctgagttaagtaagtcaatcagagaaagacaaacattatatgatctcattcatttgtggaatataaaaaatagtgaaaggggaaaggagagaaaatgagtgggaaatatcagagtgacagaacatgagagactcctatctctgggaaacaaacaaggggtaatggaagataaagtggacaggggatgtggtgactgggtgacgggcactcagtgggggcacttgataggatgagcactgggtgttatgctatatgttggcaaattgaactccaataaaaaatataccaaaaaaataagaagaaaatttggcctgttagggtcaagtggacccatagggacacagaagcattctagaccatcattaaaggattaagaaaaaagcaaacgagtgagatcagcagttactgaagcctcttttctcaaactgaagtggcaggatcagatccagcttcttcttctttttaaaaataactttgttgggatgctctcacttatgtgtggaatctaaaatagtaatatcccaagaagcagagatagaatggtggttgctgggggtgcaactcagggaataaggagactatcaaatggtacaaaagttcagttatgcaagacacacgagttctgaagatctcatgagtgccatgatgatcatagttaacaatactgtatgctaaaaatctgctaagacacagattgtgcttcttgaacttgactttccagactcaactggctgaggccagtgaggcacagtcttccttatatcaattcccagctggcccctccagtccctgactccctcccccttgcttctgggctttagtgaagttgtctggtttcacctgttacctgaaacggcctccatgtcccctgacatcatccaggagatgaggggccccagtggaccacacatgtggataataaacacaatagtcctccatgtcttcttctagtttggaagttcttatcctcagagacacacttcactgcttccattgtatgtaaagtcttacatccaaacagtaaagtgaccctggcatgagaacaccactttctagggaataaccacatggttgaggagcttagatcgattccccccagttcagccctattccattctggatgttctctgggatggctttagtcttccagtgccatgcccctgcatggacctccttccaaaggcgaggtgtaatggtctgtcactctaggcattgccttatgtcttctggtaaagaatcttggttgtgacagccaaaaagcaggaatattcccatgaaatactgcagacacagtgaagagagacattcttttttgaggaacttagtctggtctttagttcacttataattaagatgctgaagaaaatgtataatgtattttatctaccctgcacaagatcgccaaaatgtactctcgtatatctctccaccctgtctcatcttgtgatacagtgaagctaacagcagtgatagataatatttattgagtgcattttatttttcaggcactatgctaaatacttcacagaatctcatttaattaccaaactattcccatgtgttagattattatcatccccttaacctgagctgtgctttctttcccactgtcactcagctagtaaggtgatagaatttgaagtcacttctgaccccaaggccacagttatttacactctcaatgggaaggcatgcactataatgtggtgattaactcaagtcacatgaaaaaacatttattaagtgtttgtggtgtgaaattctgtgctaagtgctgtgatctgaaacagcagaaagacagtgtgctagctggctaaggcggctataaccaagtgccatggccatgggagcttgagcaatagaaatgcatttcctcaccactctggaggctggaagtctgagattgaggtgttggcagggctccttcggtggtgggatctgtcccaggtccctctccttggcttgaacatggacattttctcttattttccacattgctaccctctgcacatgtctgtctaaatttcctgatcttataaagatgcttgtcttattgattcagggctgggataaagcagaccaaatgtgagtgggatgtagggaaggacagcccatgtggggtctctgatgatgctaatgattttgtgtttatccaacaaccaaatggtttcaagtgcatcacgaagcacatgaattttcattcccacccatgcttttgccataggtatgacaagaacagatgtgaattttggaaaggtgactggctccagggtgatgaactagttgaagggaagccacactcctgtcagaagaccagctgaagttactgcagtggaggaaggtcagagcccgtggaggcag

SEQ ID NO: 2 - DNA, 472 bp, Canus lupus familiaris
tgtgcatggttggtccagatttggggtgtacgtgactaccactgtttgtgttatacatgatcaggagaatgaacaggaagaatatggaatccacttaacacatgtggtgtcgccaggaggagcatgattctgccagcattgactagacttatctctgatatgttttcaatctgcaaaactgagaagctagtaaatttgtatcaatttagactcttttcttcaatgagaagtgagttgaagaagtggcactcagaaattacctttgagttatccagctgctggaaatctaagagctgtccagaggcaagaaatacttaaaacttagagtggttaaggtatgacaagaacagatgtgaattttggaaaggtgactggctccagggtgatgaactagttgaagggaagccacactcctgtcagaagaccagctgaagttactgcagtggaggaaggtcagagcccgtggaggcag

SEQ ID NO: 3 - DNA, 401 bp, Canus lupus familiaris
tggccagctctccaccagagcgtcatccttggagatccagccaagggaaggagagagaccaagaagcaagatccctaagtgaaggttgggcgctagaaaaaaatgtcccaggtagcagtagcaccttctctttgccctctgtccttttccatctagacaacccattccgagcagagaccttgacagtcgtgttccccagcraaccaaatacaggggacatcctttatccctgaaagtgtccccttgaacaatgtaggggacagagcagacactaggaaatatttctgagcaaacaagaatgatagagcaaatagatgaatgcctgagtatctgcctggcccagaaagcagctgtaggagagaatggcccgagaaccccccatccccaccctccacagactg

SEQ ID NO: 4 - DNA, 401 bp, Canus lupus familiaris
ccctatttttcttaactttcctatttcatttatttctgattacctcgggcgcttgagttcatgttaactagtttcaactgtttaaaaaattaattttgtggaaaatatgtgtctgtgtgtgtttcaataattagttgctggacaatgagaagaacgaagattgaagctggcaatgactggttaagaaactgctaagtggtwctgctctttggaatgtatggggttgagaagcagtaaataagaactcttcatgattttcagggtcatgggctactattaaccatcattccaaagaccttcagtggctggagctttattcttttttttctctcttttttaaatttattttttattggatttcaatttgccaacatatagcataacacccagtgctcatcccg

SEQ ID NO: 5 - DNA, 401 bp, Canus lupus familiaris
aataaaatatatggcagaccattcatttaatgtagcctttgaaaagagaaaacacagaggcaactaaacggaagcatgaactgaacattccactttccgtgaggtggagggggctgaggtgcactcggcaaaagctgatagagtcaatggaacaagctctgaattcagatgctcttacttgacattaggacagcagtgtcyaggacaccaatacatgattccctcctatggtagctcatgtccaaagacagtcgcaatgaaccatgattatacagtcctctctgctttaatcagcctggtcctgtgactctcttctaatcaatagaattttatagaagatgctagattcaaaactctgcagcttccaacttgatcttttagcatgattgcactaggaaaag

SEQ ID NO: 6 - DNA, 401 bp, Canus lupus familiaris
accctccccaaatcgaactcaaaacaaaacaaaacaaaacaaaacaaaaaacccagaaaggactgccctggaagtccttgtctgattttcccaaccttttccttccaggtgtataccaaatggaccaggcttccgtgacttgcccacaagtgcccccaaacatggtcatcagggagtatcccagctataaagagaatatcrtttatgctgggcctacttcccgagcctctcaatccactgctaatgtccagcgtaagaattattcaacctcactcagtttgagccatggtgttttttatggttctacacctatcctaaaagtaaatttgcctcatttcttgctatcctggctagtcttcaatccttcctctcagaagactactgagtaagaaactctttca

SEQ ID NO: 7 - DNA, 401 bp, Canus lupus familiaris
gaaatttttcatgttgactatttcaataaatgatttagggctccttaatttgtaaggtacaaagatacgcttgaattactgggaggataaggatgtttattttcagaatattaagcggaaaacttccaaatatcaaaaaggagttataatagcttttaaactgaaatttagaaatacccagataaatgaaattataagatrtagatgtatttatgtatttaatcaattgtttcccagacccatattattgattcttcagttttgaacaacttctaaagattcttgagtttctgtttcctcatctagagaatgtttatatactccactcactgtgaggtttacatgataactttaatttaaaaacaaacacacaaacagctcattgtctattataaatgcta

SEQ ID NO: 8 - DNA, 401 bp, Canus lupus familiaris
atggaaagctatgtctatttttgaacattagctttttttcactaatttttaatcgcagactacttttccctgaatataacaagtagaaagtagttaattttcctgtagaaaggtctctgaagatttcactcatcctttcttgtctctctaatctcccaaatgactggaaataatgaaggaagagatgctggtactcctctkgggtaatgatctcatgacttccaagggtgggtttgccgtcataatagttggggacaaaagtcagggtggtaactgggggtcgaataagatctggcaatcttgagaatgtgaagtaatttggaatatagcatgctcacagagtgggtattttatcctgttgggtagaaaagaccatcaggtgtgctggtgtctgttactct

SEQ ID NO: 9 - DNA, 401 bp, Canus lupus familiaris
cagggtggtaactgggggtcgaataagatctggcaatcttgagaatgtgaagtaatttggaatatagcatgctcacagagtgggtattttatcctgttgggtagaaaagaccatcaggtgtgctggtgtctgttactcttgggagcccctagtttaaagactaggaggctagctcagagttgaggagatgcctttttttcmagagtaagaataattttgattttcagtatttccagtggagcagatccctccagaaaagtcgtgagagctgcatgtcaattgctgttatcccaatggtatttagaacattttcttgattaattacaagaaaatttggcctgggagggggctgcatcctgctgctttcaggagccatggcctgggagcacaattccagcagt

SEQ ID NO: 10 - DNA, 401 bp, Canus lupus familiaris
cgtgacctgagctgaaatcaagatttggtggcttaaccaactgagcagcccaggtgccctgacagtgagttgtaaagcaagaaagcaagcttgtgattagtccagtgagcctgttaagtagaggttcattaaaagagcttgtatgaactgaattatagagtatgccatggctttctcttgcagaaaatatgaagtgcatayaactatgaaatgtggctacttctatgcaagaatgacattcagaataaaattattcatcaacaacagctatccagatgttttagtttgtaaagacataattttaatgaccgaatatttcagttttcatttgaatgttccaatttttttgaatacgccatttcattttcagtgctaaataaatacttgttcagtttttaaaa

SEQ ID NO: 11 - DNA, 401 bp, Canus lupus familiaris
ctcagcagatgcatcacatttggcgcacaagtcccaagtcaggtccagtgaccaacggaggaagacccttgagggctaggattgtgcctactgttctcctacctgttggctccacctgcactcataccaagcacttctctgaccctcgctgccaccagctctgccccatcctggttgccgtcatctctgaagactggcagracccatgagacttaagaactttcccaagcttctgctttgcattggcctaaatctctgtaatttaaaggacttcttccaactcccattccctgacctcatctggtgtcactttatcatccaacataaacactatttctcacctcagctccccacacactgtttctctcttgtgtggctttatttctgtttcacttattctc

SEQ ID NO: 12 - DNA, 401 bp, Canus lupus familiaris
atcaacaaggagtgtagaaggaaaaaattcaggtgaggggacagccagtgaactgcgctgacattctctctggagatgtgtgatcctcagggtttgtgctgagcctggcctccccaggaagaggactgatggatgtgcagagagaaatcaacaaatactccagtttgtacaaagttgaagactgaggggccattaagacayagctgctttttgcctaaaaccttttgctaaattttgaaattgtctgtgtcaagaataggatatattttccccttgagagtcgtggtgtatactaacaagtgctttgagaaatctttctgagacaaaagctgtttaacttctgtatcctgtattttcccaaacttacttgcttaaggtgttcttaattcataacaaaagac

SEQ ID NO: 13 - DNA, 401 bp, Canus lupus familiaris
tatgtggctgtcactacagcaatgacttgtttttggcatatgtcacatcatttcagagaaacaaaaattaatctgaaatctttccttaaggattaattctatgtatataagaaggaaagtcaaggcaatggaagcaggcaaatttaggcaataaggatcctgggattagtgagagaatgtccaacaaatcctccaagggakgctcaaagcaataggctctgcttcagcaggatggtaaaggtcacccttgcttatttttgctgcttcatggagagaagcagtagacttaaatatgtttaagggcttaaaacagaatgaagttgagactgtctgggttatctgactgctggacttcttcagtgctgctggattctaaacagagtccatctgtgtcaagtgtt

SEQ ID NO: 14 - DNA, 401 bp, Canus lupus familiaris
tttaacttctatgcttcaaaatctttacagtccatgagaaaagcacagcagaagttaaagctacccagggattcccagatgaggacctattaactgtgagaatgtgctcctttgttatgtttctctcagagagtgagtccacctcaggtttccagagtgtggatcccctcccccgaatcacagcggctgcttggggtctgkaatcccccatccactctgtaggcaaaaaccctgttaagcaatgtgggggacacagcagccagtggggggtttgtcttaggttagggccccagatctgatcatcttacaagtcttcttgacatatacagtaaatacatggctttgctttcaggccaggaaaatcttgagaacacatgtcaatattttgtagaaaattattt

SEQ ID NO: 15 - DNA, 401 bp, Canus lupus familiaris
aaatatttttctttattatttcagcttttaggggaatacttagaatggcattatacacctgaagattacatattaaaaaataaaagttcacctgactctttctctagaggttttatggtttttaaaatgacattcaatttcttaatgcatctcatttactttatggcaaaggtagagaaagaaatctcttacccacttccytttattcagtatggcttcattttccccaatgagttatgtcctttatcataccataaaatcttatatccttaagtctttgactgaactttctattatattcttttaatgtttccattttgtgaatacccaactatttattgtggttttactaaatcatttaatgtcttatagagcaagtgcctttcactgctttttaaaaa

SEQ ID NO: 16 - DNA, 401 bp, Canus lupus familiaris
gttcatttaatttaccaaagttaacattattcactttacagcatatgtagaaaattgaggtccaggctgtatttgactacttccagtgataagaaaataactacatataaggcagttccatccattcttgatatgtctgcatttcctgagccagggtgactccactccataactcagcagtgctctcaactgtcctctgaytgtttatgaaccattcctctgttacccagtctgtatttgttaatcttgctgccaaatatattacatgatgcccttggtcagtagtttactgaaaggattctaatcattctaccaaaagacaagttaatttagtccagcatgacttatcctaagcaaagccgcggtgatcacagtgcttacacaatatccgtataataata

SEQ ID NO: 17 - DNA, 401 bp, Canus lupus familiaris
ggttctcagtcttgctctcccctctgttgcaggtgagctgagcctcagggtctggaagcctcttctgcctcccctgctcctattccccattatcttccagaggcatgattctggatacatctcatacttctaactgtcttggcgtttggttcctagaagaccaaagtaacacaactatcctctttactttgcttggccccrtaagcctgtgatgaacaaaacttggggcacgtgccagacacgtattcctgggagaatgttttttaaagcaactgtttatatttcaaatcatttttgcctattgtggactcccactggaaatgtgtggctctgacaggtaccaaggaaagttatgcgcccttacccaacgtgggtagcttttgctttctttttcataaagt

SEQ ID NO: 18 - DNA, 401 bp, Canus lupus familiaris
gaccctgtgactctccttcctgacagaagccttggggaaggcccaagacgggaaggaagaagcaccagcgaggccaaggacagcaggaaggagctagaatgattgcagcttcggccgcgatccgctggatgcagacgggccggctgtgacactcccttccccgcatcacaggtcctgatcttggaccacagccgcatctcyaatgcatggtgcatccaaaggaggtgctcggtcatcacatgtggctcaccacggcagcctgccctcccagagggtgtcctggaaccggccctctgcagagctggtttcaaagcccgggcagcccctctgcgagccgccttcctccggcacggtgggtgaggaaatgagagagggaatgtctaatgattggttccttatgg

SEQ ID NO: 19 - DNA, 401 bp, Canus lupus familiaris
tgggcctcacccatagaatggggatcatagtagtatgtatatttgttgggtatggtacctataacccagtcatgctcagtaaacatctgcttttcccattactagggcttcaccaggcatgtttcatggtgtgcctatagtcccttgaaatgggctctttgttgacctagactctggttgagggcaagccctggcagctgyggtttttacctcatgatcctacccattgagccatggtgacttgggcacatagaggtgacccaacccatgggctggccagcagtttctgactcagcccatgaagctgagttgagtagaaagattcttttctctttttgaactaggaaataaggagccatgcagacttgctagatgtccttaggataaattcaccttttttg

SEQ ID NO: 20 - DNA, 401 bp, Canus lupus familiaris
ggctctgtgctcctagaccatacttgtggaaatcactaatgatgtatgctatagctcctaccaactgtggaacataactggtaagtccttctggagtgtggaagtgagagaaatcactggcggccgaggcactcagatttgacaggactaggccaagagattatattctgggctgaactgcaagattgagaggcaggaggragaggcacattctggactgggccaaagacacaagaaacaagcaaaggtgaaaggaaggaaacgggcctggcacattgggctgagtggccctaggtaggaggatacagtatcacagggtcacagatgcggggaagctagtttctgcagagactcgaacaccaggatcatcgggtgagacttgacaagagggctttagagat

SEQ ID NO: 21 - DNA, 401 bp, Canus lupus familiaris
gcccaaacttttttttaattttattttatttttttttaaggacagtctgttattctagatctgctttaatttcatgcaacagtgataactaagagtaagtaagtactcgtaagtaagatttctggtatggcacccacagtaccctccatgttggccccagtttcagatattccattatattgtccctcatgagagatcctrcaaattccaatttgacatccaagtgacatctaccaggagggccttggagaagctgatttttctttttaattttgaaagtactcaaaataagaattcaaatgagaagtatatttttattcaaatgagaatgtgtacaaaatagttatcgagcacttattatgtgcataacactggaggccaaaaaaaaaaaaaaagaggaa

SEQ ID NO: 22 - DNA, 401 bp, Canus lupus familiaris
caggagcccaatgcaggactcaatcccaggaccccaggatcatgacctgagcccaaagcagacgttcaaccattgagccaccctagagtccctgtgtctccttttcttgtcttgtgttgtgtcgtgatcatgttttgtggttgtaccttcccctccctgacttcacatgacttggaaactattcatggtattgtttgttakttatcaatctttaagtcataagtatgtatatttgatataataatttatgattatgatattgtttctagttctttctagatattgcctgtctgttaattcattggtatcatagtttcctttacatttttaaatattttatttgtttatttgagagagagtgagagacagagatagtgagagcatgaacaaggaggaaaggg

SEQ ID NO: 23 - DNA, 401 bp, Canus lupus familiaris
tgatacatttcttacaggatggttttgtcatgtagaagctcttttaaagcactccatccttattttcccattgatcatttctttgcctccttttcccccttctctcctctagaaatgtccctttttctctcaccattatcagcacccattaaccttctaagtaacacaattgattttgacctctctttgtggttttaattyaccataggtgtcaggggtgtcatctttcttttctgtttcccctgtgttccagcctgcttgagggtgaatgccctggcagggtgtgcccacatgcactcacatataacccatagacagcaactccaggaacatatcaaactggatttcttaagtcactggagcctatgggtgactgtacgtatagacaatatattttgaat

SEQ ID NO: 24 - DNA, 401 bp, Canus lupus familiaris; identical to SEQ ID NO: 3

SEQ ID NO: 25 - DNA, 401 bp, Canus lupus familiaris; identical to SEQ ID NO: 4